(19) United States
(12) Patent Application Publication
(10) Pub. No.: US 2025/0084060 A1
(43) Pub. Date: Mar. 13, 2025

(54) PRMT5 INHIBITORS AND METHODS OF TREATMENT

(71) Applicant: HANGZHOU UNOGEN BIOTECH, LTD., Zhejiang (CN)

(72) Inventors: Ao Yu, Shanghai (CN); Ho Yin Lo, Bethel, CT (US)

(21) Appl. No.: 18/952,350

(22) Filed: Nov. 19, 2024

Related U.S. Application Data
(63) Continuation of application No. PCT/IB2023/000285, filed on May 18, 2023.
(60) Provisional application No. 63/344,413, filed on May 20, 2022.

(51) Int. Cl. C07D 401/06; A61K 31/517; A61P 35/00

(52) U.S. Cl. CPC C07D 401/06; A61K 31/517; A61P 35/00

(57) ABSTRACT

The present invention relates to protein arginine N-methyltransferase-5 inhibitors and more specifically to substituted quinazoline-2,4 compounds useful as such agents. These inhibitors are useful as anti-cancer agents.

### CROSS-REFERENCE TO RELATED APPLICATIONS

This continuation application claims the benefit of and priority to International Patent Application No. PCT/IB2023/000285, filed May 18, 2023, which claims priority to U.S. Provisional Patent Application No. 63/344,413, filed May 20, 2022, each of which are incorporated by reference in their entireties herein.

### FIELD OF THE INVENTION

The present invention relates to protein arginine N-methyltransferase-5 inhibitors and more specifically to substituted quinazoline-2,4-dione compounds useful as such agents. These inhibitors are useful as anti-cancer agents.

### BACKGROUND OF THE INVENTION

Cancer is a common cause of death and morbidity worldwide. An unmet need remains for new and effective methods, systems, and compositions for the treatment of cancer. The etiology of and the biochemical pathways of cancer development and progression are complex, but represent a huge potential for the development of new targeted cancer therapies.

Protein arginine N-methyltransferases (abbreviated “PRMTs”) are a family of enzymes which catalyze arginine methylation and which is involved in cell regulatory functions and cancer development. There are at least nine PRMTs known in mammals. Protein arginine N-methyltransferase-5 (abbreviated as “PRMT5”) is the major enzyme responsible for the mono-and symmetric dimethylation of arginine, and is more specifically involved with arginine methylation of histones, which are proteins associated with DNA that provide the DNA with structure and integrity. PRMT5 plays a key role in cell regulatory pathways, including those involved with cancer development, progression, and therapy response. Because dysregulation of protein arginine methylation is closely associated with cancer development, PRMTs are potentially an important target for anti-cancer drug development.

We have synthesized a collection of substituted quinazoline-2,4-dione compounds as inhibitors of PRMT-5 for anti-cancer therapies. We evaluated these compounds using a PRMT5 chemiluminescent assay. The results from these evaluations indicate the potential of substituted quinazoline-2,4-dione compounds as anti-cancer therapies for further developing a new class of anticancer drugs.

It is apparent from the foregoing that there is an ongoing need for developing safe and effective compounds as new therapeutic agents for treating cancer. It has been found in the present invention that substituted quinazoline-2,4-dione compounds can be useful for treating certain types of cancers.

### SUMMARY OF THE INVENTION

The present invention provides substituted quinazoline-2,4-dione compounds or pharmaceutically acceptable salts, esters, solvates, or prodrugs thereof.

In an embodiment, the present invention provides for compounds having the following formula

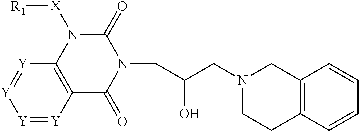

wherein
X is C¹-C⁶ alkyl, CO, or a bond,
Y is, at each occurrence, is independently N, CH, or CR².
R¹ is alkyl, heteroalkyl, aromatic, heterocycle, carbocycle, heteroaromatic, spirocycles, heterospirocycles, amide, alkoxy, or fused or bridged bicyclics or heterobicyclics, optionally substituted at any position with one or more R³
R² is H, halo, alkyl halide, alkoxy, alkyl, alkenyl, alkynyl, heteroalkyl, heteroalkenyl, heteroalkynyl, amino, alkyl amide, alkyl carboxylic acid, and cyano, or a pharmaceutically acceptable salt, ester, prodrug, or solvate thereof

wherein R¹ optionally has the formula A¹-L¹-B¹-, where A¹ is a first aromatic, heteroaromatic, carbocycle, heterocycle, spirocycle, or bridged or fused combinations thereof, where L¹ is a linker selected from alkyl, heteroalkyl, CO, O, N, or S, and where B¹ is a second aromatic, heteroaromatic, carbocycle, heterocycle, spirocycle, heterospirocycle, or bridged or fused combinations thereof, wherein B¹ is attached to the N of the remainder of the compound,

wherein R³ is selected from H, halo, hydroxy, —C═OR⁴, alkyl halide, alkoxy, alkyl, alkenyl, alkynyl, heteroalkyl, heteroalkenyl, heteroalkynyl, amino, alkyl amide, alkyl carboxylic acid, and cyano, each optionally connected through a C₁-C₆ linker, and

wherein R⁴ is H or a C₁-C₆ alkyl, C₁-C₆ heteroalkyl, aryl, or heteroaryl, or a pharmaceutically acceptable salt, ester, prodrug, or solvate thereof.

In some embodiments the present invention provides a compound of Table 1, or a pharmaceutically acceptable, salt, ester, solvate, or prodrug thereof.

In some embodiments the present invention provides a pharmaceutical composition comprising a substituted quinazoline-2,4-dione compounds or pharmaceutically acceptable salts, esters, solvates, or prodrugs thereof.

In some embodiments the present invention provides a pharmaceutical composition comprising a compound of Table 1, or a pharmaceutically acceptable, salt, ester, solvate, or prodrug thereof.

In some embodiments the present invention provides methods of treating cancer with substituted quinazoline-2,4-dione compounds, or pharmaceutically acceptable salts, esters, solvates, or prodrugs thereof.

In some embodiments the present invention provides methods of treating cancer with a compound of Table 1, or a pharmaceutically acceptable, salt, ester, solvate, or prodrug thereof.

In further embodiments the present invention provides methods for treating a subject or patient in need thereof.

In further embodiments the subject or patient is a mammal.

In further embodiments the subject is a human.

In further embodiments is provided a composition or unit dosage comprising from about 1 μg to about 1000 mg of a compound of the present invention.

In further embodiments is provided a composition selected from the group consisting of a solid tablet, a capsule, a liquid, a suspension, a gel, an ointment, a solution for infusion, a suppository, an inhalable composition., and an injectable composition.

In further embodiments the compound or pharmaceutical composition is administered from the group consisting of about four times per day, about three times per day, about two times per day, about one time per day, about one time every other day, about two times per week, and about one time per week.

In further embodiments the compound or pharmaceutical composition is administered for a period of treatment selected from the group consisting of a single dose on one day to a course of therapy spanning multiple days such as from 2 days to about 6 months, or from about 2 days to about 3 months, or from about 2 days to about 1 month, or from about 2 days to about two weeks, or from about 2 days to about 10 days, or from about 2 days to about 1 week.

In further embodiments the present invention relates to the use of a compound of the present invention or a pharmaceutically acceptable salt, ester, solvate, or prodrug thereof, in the manufacture of a medicament for treating cancer.

In an embodiment, the present disclosure provides for compounds having the following formula

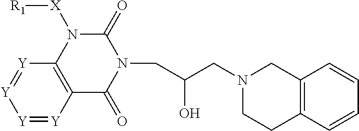

wherein
X is C₁-C₆ alkyl, CO, or a bond,
Y is, at each occurrence, is independently N, CH, or CR².
R¹ is H, C₁-C₆ alkyl, C₁-C₆ heteroalkyl, phenyl, C₃-C₇ heterocycle, C₃-C₇ carbocycle, C₅-C₆ heteroaromatic, C₈-C₁₁ spirocycle, C₈-C₁₁ heterospirocycles, amide, alkoxy, or C₇-C₁₂ fused or bridged bicyclics or heterobicyclics, optionally substituted at any position with one or more R³
R² is H, hydroxy, halo, C₁-C₆ alkyl halide, C₁-C₆ alkoxy, C₁-C₆ alkyl, C₁-C₆ alkenyl, C₁-C₆ alkynyl, C₁-C₆ heteroalkyl, C₁-C₆ heteroalkenyl, C₁-C₆ heteroalkynyl, amino, alkyl amide, alkyl carboxylic acid, pyridyl, methoxy pyridyl, phenyl, fluorophenyl, and cyano, or a pharmaceutically acceptable salt, ester, prodrug, or solvate thereof

wherein R¹ optionally has the formula A¹-L¹-B¹-, where A¹ is a first aromatic, heteroaromatic, carbocycle, heterocycle, spirocycle, or bridged or fused combinations thereof, where L¹ is a linker selected from alkyl, heteroalkyl, CO, O, N, or S, and where B¹ is a second aromatic, heteroaromatic, carbocycle, heterocycle, spirocycle, heterospirocycle, or bridged or fused combinations thereof, wherein B¹ is attached to the N of the remainder of the compound

wherein R³ is selected from H, halo, hydroxy, acyl, —C═OR⁴, alkyl halide, alkoxy, alkyl, alkenyl, alkynyl, heteroalkyl, heteroalkenyl, heteroalkynyl, amino, alkyl amide, alkyl carboxylic acid, and cyano, each optionally connected through a C₁-C₆ linker, and

wherein R⁴ is H or a C₁-C₆ alkyl, C₁-C₆ heteroalkyl, aryl, or heteroaryl, or a pharmaceutically acceptable salt, ester, prodrug, or solvate thereof.

In an embodiment, the compound is selected a compound 1-105, or a pharmaceutically acceptable salt, ester, prodrug, or solvate thereof:

In an embodiment, the present disclosure provides for a pharmaceutical composition comprising one or more compounds of the present disclosure, or one or more of compounds 1-105.

In an embodiment, the present disclosure provides for a method for treating a disease state or condition in a subject or patient in need thereof comprising administering one or more compounds of the present disclosure, or one or more of compounds 1-105, or by administering a pharmaceutical composition of the present disclosure.

In an embodiment, the subject or patient in need thereof is a mammalian subject.

In an embodiment, the mammalian subject is a human.

In an embodiment, the disease state or condition is a cancer.

In an embodiment, the cancer is lymphoma.

In an embodiment, the present disclosure provides for a method of treating a cancer comprising administering to a mammalian subject in need thereof one or more compounds according to the following formula:

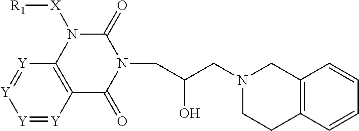

wherein
X is C₁-C₆ alkyl, CO, or a bond,
Y is, at each occurrence, is independently N, CH, or CR²,
R¹ is H, C₁-C₆ alkyl, C₁-C₆ heteroalkyl, phenyl, C₃-C₇ heterocycle, C₃-C₇ carbocycle, C₅-C₆ heteroaromatic, C₈-C₁₁ spirocycle, C₈-C₁₁ heterospirocycles, amide, alkoxy, or C₇-C₁₂ fused or bridged bicyclics or heterobicyclics, optionally substituted at any position with one or more R³
R² is H, hydroxy, halo, C₁-C₆ alkyl halide, C₁-C₆ alkoxy, C₁-C₆ alkyl, C₁-C₆ alkenyl, C₁-C₆ alkynyl, C₁-C₆ heteroalkyl, C₁-C₆ heteroalkenyl, C₁-C₆ heteroalkynyl, amino, alkyl amide, alkyl carboxylic acid, pyridyl, methoxy pyridyl, phenyl, fluorophenyl, and cyano, or a pharmaceutically acceptable salt, ester, prodrug, or solvate thereof

wherein R¹ optionally has the formula A¹-L¹-B¹-, where A¹ is a first aromatic, heteroaromatic, carbocycle, heterocycle, spirocycle, or bridged or fused combinations thereof, where L¹ is a linker selected from alkyl, heteroalkyl, CO, O, N, or S, and where B¹ is a second aromatic, heteroaromatic, carbocycle, heterocycle, spirocycle, heterospirocycle, or bridged or fused combinations thereof, wherein B¹ is attached to the N of the remainder of the compound

wherein R³ is selected from H, halo, hydroxy, acyl, —C═OR⁴, alkyl halide, alkoxy, alkyl, alkenyl, alkynyl, heteroalkyl, heteroalkenyl, heteroalkynyl, amino, alkyl amide, alkyl carboxylic acid, and cyano, each optionally connected through a C₁-C₆ linker, and

wherein R⁴ is H or a C₁-C₆ alkyl, C₁-C₆ heteroalkyl, aryl, or heteroaryl, or a pharmaceutically acceptable salt, ester, prodrug, or solvate thereof.

In an embodiment, the cancer is lymphoma.

In an embodiment, the mammalian subject is a human.

In an embodiment, the one or more compounds for treating a cancer or lymphoma are selected from compounds 1-105.

In an embodiment, the present invention provides the use of a compound of the present invention in treating a disease state or condition in a mammal in need thereof.

In an embodiment, the present invention provides a compound of the present invention for the manufacture of a medicament for treating a disease state or condition.

These and other aspects of the present invention will become apparent from the disclosure herein.

### BRIEF DESCRIPTION OF THE DRAWINGS

FIG. 1 shows a plot of demonstrating PRMT5 activity for compound 1.

FIG. 2 shows a plot of demonstrating PRMT5 activity for compound 2.

FIG. 3 shows a plot of demonstrating PRMT5 activity for compound 3.

FIG. 4 shows a plot of the mouse PK data for compound 70.

### DETAILED DESCRIPTION OF THE INVENTION

### Definitions

As used herein, the following terms and abbreviations have the indicated meanings unless expressly stated to the contrary.

The term “compound capable of acting as an inhibitor of PRMT5” means a compound demonstrating activity of about 10 μM or less, as based on an IC50 using a PRMT5 chemiluminescent assay.

The terms “effective”, “pharmaceutically effective”, and “therapeutically effective” means an amount of a PRMT5 inhibitor needed to provide a meaningful or demonstrable benefit, as understood by medical practitioners, to a subject, such as a human patient in need of treatment. Conditions, intended to be treated include various cancers. For example, a meaningful or demonstrable benefit can be assessed or quantified using various clinical parameters. The demonstration of a benefit can also include those provided by models, including but not limited to in vitro models, in vivo models, and animal models.

The term “pharmaceutically acceptable” is used herein with respect to the compositions, in other words the formulations, of the present invention, and also with respect to the pharmaceutically acceptable salts, esters, solvates, and prodrugs thereof. The pharmaceutical compositions of the present invention comprise a therapeutically effective amount of the active agents and a pharmaceutically acceptable carrier. These carriers can contain a wide range of excipients. Pharmaceutically acceptable carriers are those conventionally known carriers having acceptable safety profiles. The compositions are made using common formulation techniques. See, for example, Remington's Pharmaceutical Sciences, 17^{th} edition, edited by Alfonso R. Gennaro, Mack Publishing Company, Easton, PA, 17^{th} edition, 1985. Regarding pharmaceutically acceptable salts, these are described below.

The term “subject” means a human patient or animal in need of treatment or intervention for a coronavirus infection.

The abbreviation “PRMT” as used herein refers to protein arginine N-methyltransferases. The abbreviation “PRMT-5” as used herein refers to protein arginine N-methyltransferase-5. The terms “treat,” “treating” or “treatment,” as used herein, include alleviating, abating or ameliorating the condition, or preventing or reducing the risk of contracting the condition or exhibiting the symptoms of the condition, ameliorating or preventing the underlying causes of the symptoms, inhibiting the condition, arresting the development of the condition, relieving the condition, causing regression of the condition, or stopping the symptoms of the condition, either prophylactically and/or therapeutically.

### Protein Arginine N-Methyltransferase-5 Inhibitors

The present invention provides compounds useful for treating diseases such as cancer. These compounds have activity as inhibitors of PRMT-5. These compounds of the present invention are substituted quinazoline-2,4-diones.

Exemplary compounds of the present invention are provided in Table 1 along with their corresponding compound number. The IUPAC names of the compounds of Table 1 are provided in Table 2.

TABLE 1
Exemplary compounds

| | |
|---|---|
| 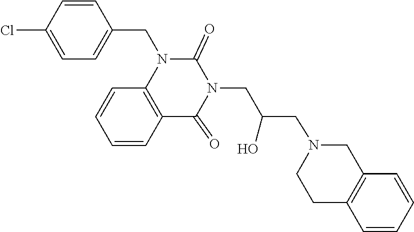 [SMILES: O=c1c2ccccc2n(Cc2ccc(Cl)cc2)c(=O)n1CC(O)CN1CCc2ccccc2C1] | 1 |
| 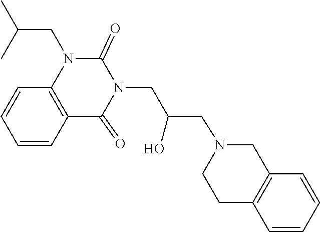 [SMILES: CC(C)Cn1c(=O)n(CC(O)CN2CCc3ccccc3C2)c(=O)c2ccccc21] | 2 |
| 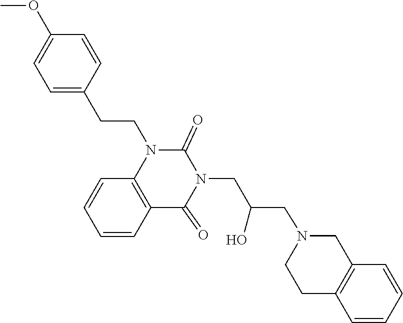 [SMILES: COc1ccc(CCn2c(=O)n(CC(O)CN3CCc4ccccc4C3)c(=O)c3ccccc32)cc1] | 3 |
| 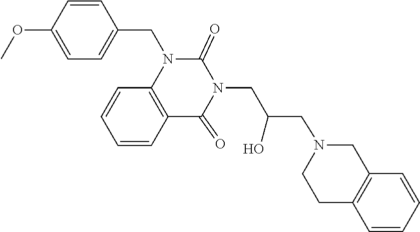 [SMILES: COc1ccc(Cn2c(=O)n(CC(O)CN3CCc4ccccc4C3)c(=O)c3ccccc32)cc1] | 4 |
| 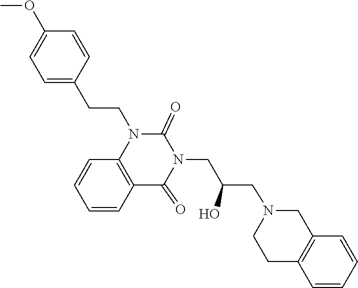 [SMILES: COc1ccc(CCn2c(=O)n(C[C@H](O)CN3CCc4ccccc4C3)c(=O)c3ccccc32)cc1] | 5 |
| 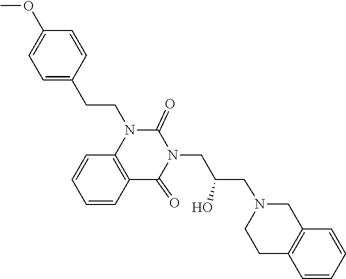 [SMILES: COc1ccc(CCn2c(=O)n(C[C@@H](O)CN3CCc4ccccc4C3)c(=O)c3ccccc32)cc1] | 6 |
| 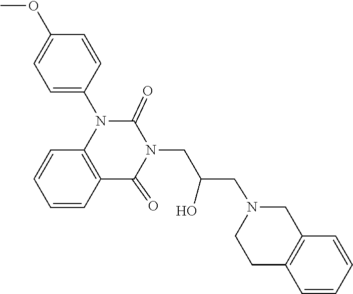 [SMILES: COc1ccc(-n2c(=O)n(CC(O)CN3CCc4ccccc4C3)c(=O)c3ccccc32)cc1] | 7 |
| 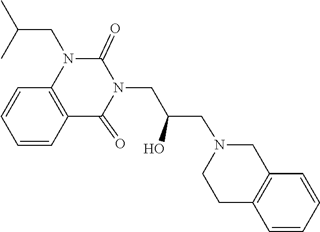 [SMILES: CC(C)Cn1c(=O)n(C[C@H](O)CN2CCc3ccccc3C2)c(=O)c2ccccc21] | 8 |
| 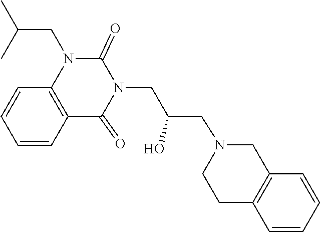 [SMILES: CC(C)Cn1c(=O)n(C[C@@H](O)CN2CCc3ccccc3C2)c(=O)c2ccccc21] | 9 |
| 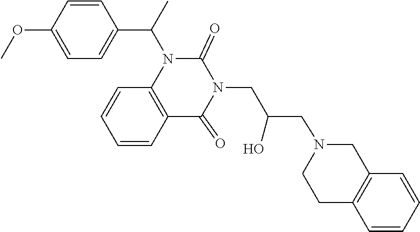 [SMILES: COc1ccc(C(C)n2c(=O)n(CC(O)CN3CCc4ccccc4C3)c(=O)c3ccccc32)cc1] | 10 |
| 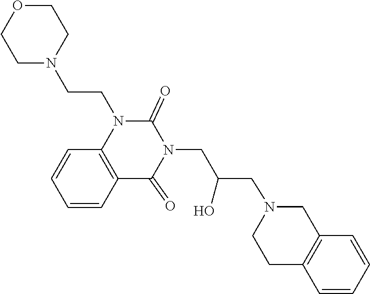 [SMILES: O=c1c2ccccc2n(CCN2CCOCC2)c(=O)n1CC(O)CN1CCc2ccccc2C1] | 11 |
| 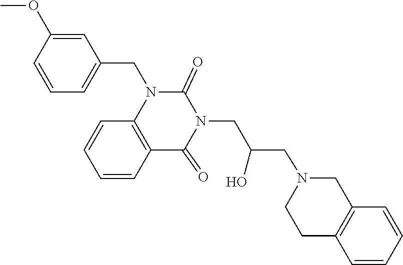 [SMILES: COc1cccc(Cn2c(=O)n(CC(O)CN3CCc4ccccc4C3)c(=O)c3ccccc32)c1] | 12 |
| 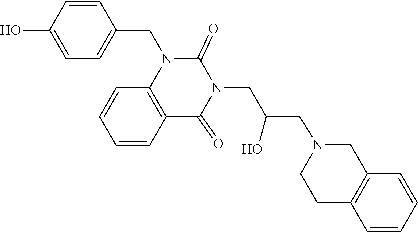 [SMILES: O=c1c2ccccc2n(Cc2ccc(O)cc2)c(=O)n1CC(O)CN1CCc2ccccc2C1] | 13 |
| 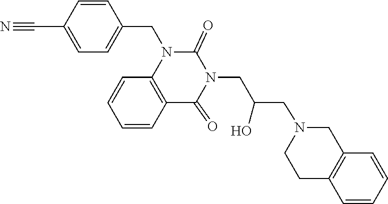 [SMILES: N#Cc1ccc(Cn2c(=O)n(CC(O)CN3CCc4ccccc4C3)c(=O)c3ccccc32)cc1] | 14 |
| 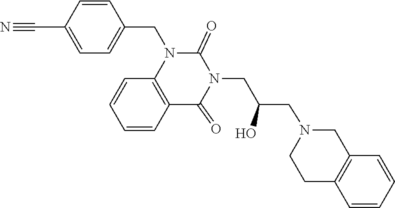 [SMILES: N#Cc1ccc(Cn2c(=O)n(C[C@H](O)CN3CCc4ccccc4C3)c(=O)c3ccccc32)cc1] | 15 |
| 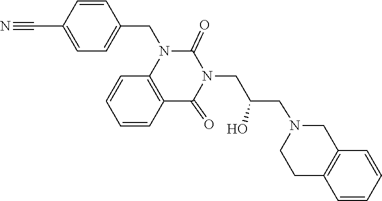 [SMILES: N#Cc1ccc(Cn2c(=O)n(C[C@@H](O)CN3CCc4ccccc4C3)c(=O)c3ccccc32)cc1] | 16 |
| 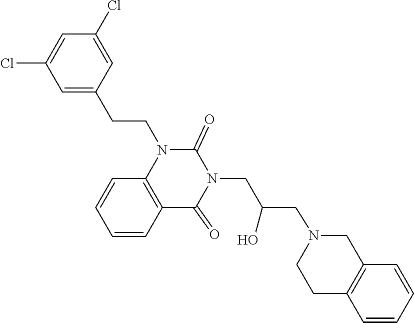 [SMILES: O=c1c2ccccc2n(CCc2cc(Cl)cc(Cl)c2)c(=O)n1CC(O)CN1CCc2ccccc2C1] | 17 |
| 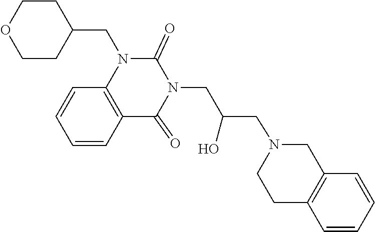 [SMILES: O=c1c2ccccc2n(CC2CCOCC2)c(=O)n1CC(O)CN1CCc2ccccc2C1] | 18 |
| 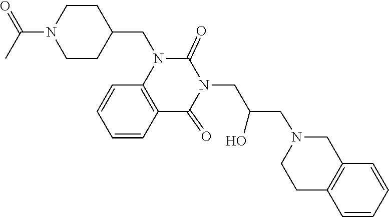 [SMILES: CC(=O)N1CCC(Cn2c(=O)n(CC(O)CN3CCc4ccccc4C3)c(=O)c3ccccc32)CC1] | 19 |
| 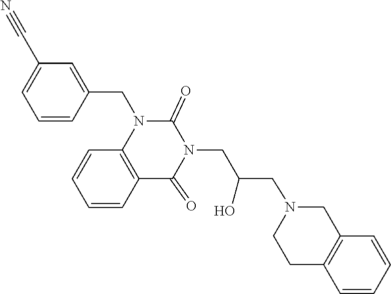 [SMILES: N#Cc1cccc(Cn2c(=O)n(CC(O)CN3CCc4ccccc4C3)c(=O)c3ccccc32)c1] | 20 |
| 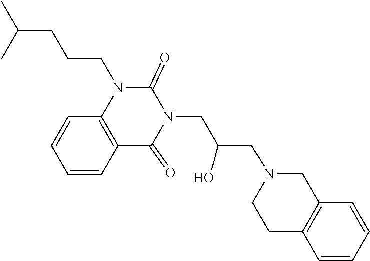 [SMILES: CC(C)CCCn1c(=O)n(CC(O)CN2CCc3ccccc3C2)c(=O)c2ccccc21] | 21 |
| 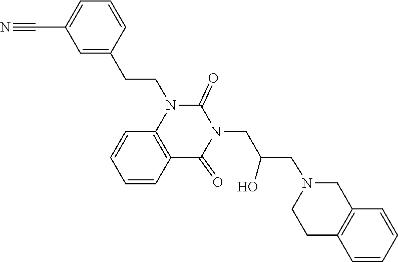 [SMILES: N#Cc1cccc(CCn2c(=O)n(CC(O)CN3CCc4ccccc4C3)c(=O)c3ccccc32)c1] | 22 |
| 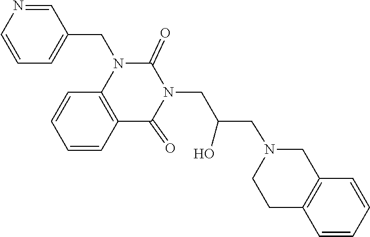 [SMILES: O=c1c2ccccc2n(Cc2cccnc2)c(=O)n1CC(O)CN1CCc2ccccc2C1] | 23 |
| 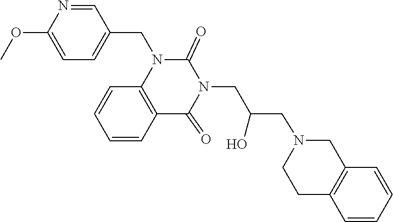 [SMILES: COc1ccc(Cn2c(=O)n(CC(O)CN3CCc4ccccc4C3)c(=O)c3ccccc32)cn1] | 24 |
| 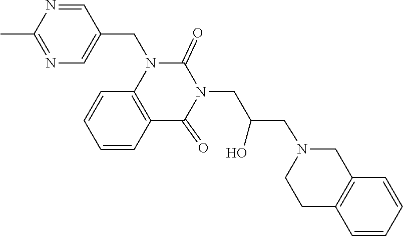 [SMILES: Cc1ncc(Cn2c(=O)n(CC(O)CN3CCc4ccccc4C3)c(=O)c3ccccc32)cn1] | 25 |
| 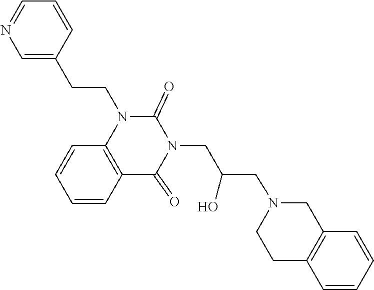 [SMILES: O=c1c2ccccc2n(CCc2cccnc2)c(=O)n1CC(O)CN1CCc2ccccc2C1] | 26 |
| 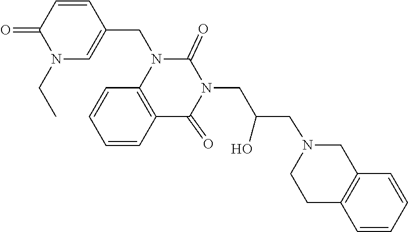 [SMILES: CCn1cc(Cn2c(=O)n(CC(O)CN3CCc4ccccc4C3)c(=O)c3ccccc32)ccc1=O] | 27 |
| 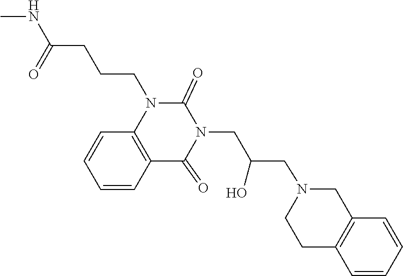 [SMILES: CNC(=O)CCCn1c(=O)n(CC(O)CN2CCc3ccccc3C2)c(=O)c2ccccc21] | 28 |
| 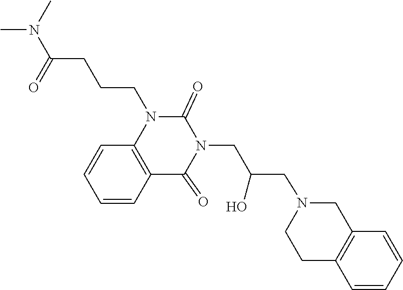 [SMILES: CN(C)C(=O)CCCn1c(=O)n(CC(O)CN2CCc3ccccc3C2)c(=O)c2ccccc21] | 29 |
| 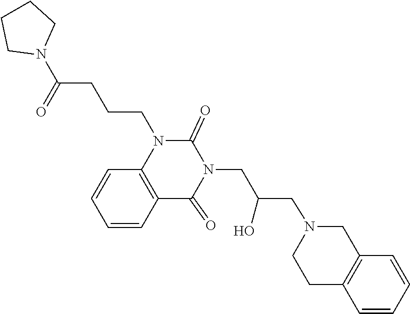 [SMILES: O=C(CCCn1c(=O)n(CC(O)CN2CCc3ccccc3C2)c(=O)c2ccccc21)N1CCCC1] | 30 |
| 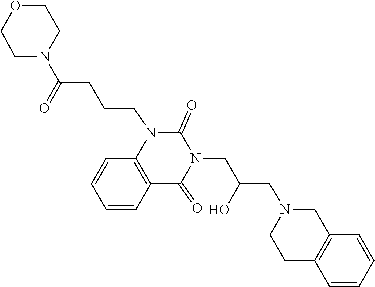 [SMILES: O=C(CCCn1c(=O)n(CC(O)CN2CCc3ccccc3C2)c(=O)c2ccccc21)N1CCOCC1] | 31 |
| 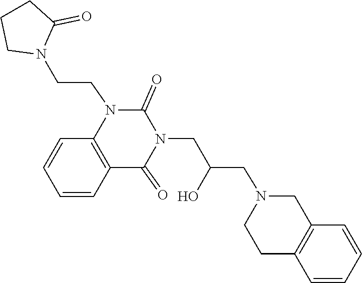 [SMILES: O=C1CCCN1CCn1c(=O)n(CC(O)CN2CCc3ccccc3C2)c(=O)c2ccccc21] | 32 |
| 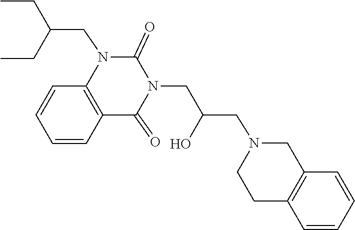 [SMILES: CCC(CC)Cn1c(=O)n(CC(O)CN2CCc3ccccc3C2)c(=O)c2ccccc21] | 33 |
| 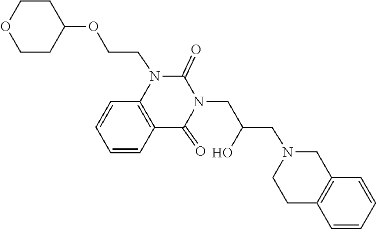 [SMILES: O=c1c2ccccc2n(CCOC2CCOCC2)c(=O)n1CC(O)CN1CCc2ccccc2C1] | 34 |
| 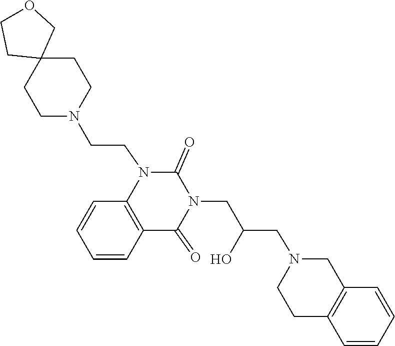 [SMILES: O=c1c2ccccc2n(CCN2CCC3(CCOC3)CC2)c(=O)n1CC(O)CN1CCc2ccccc2C1] | 35 |
| 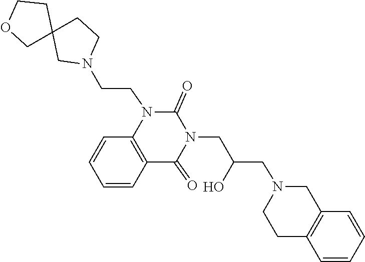 [SMILES: O=c1c2ccccc2n(CCN2CCC3(CCOC3)C2)c(=O)n1CC(O)CN1CCc2ccccc2C1] | 36 |
| 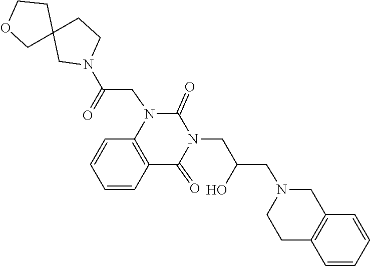 [SMILES: O=C(Cn1c(=O)n(CC(O)CN2CCc3ccccc3C2)c(=O)c2ccccc21)N1CCC2(CCOC2)C1] | 37 |
| 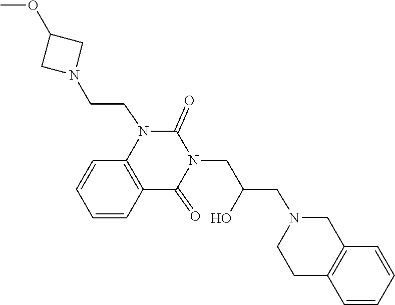 [SMILES: COC1CN(CCn2c(=O)n(CC(O)CN3CCc4ccccc4C3)c(=O)c3ccccc32)C1] | 38 |
| 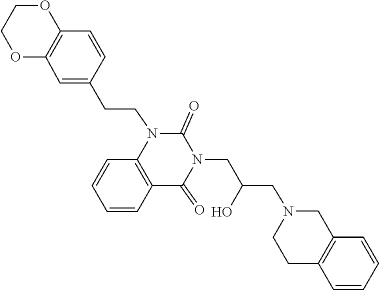 [SMILES: O=c1c2ccccc2n(CCc2ccc3c(c2)OCCO3)c(=O)n1CC(O)CN1CCc2ccccc2C1] | 39 |
| 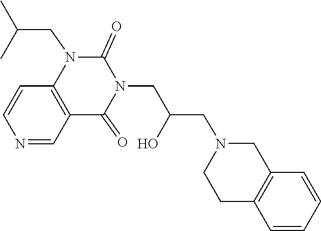 [SMILES: CC(C)Cn1c(=O)n(CC(O)CN2CCc3ccccc3C2)c(=O)c2cnccc21] | 40 |
| 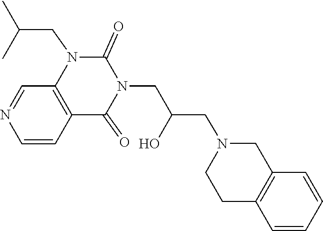 [SMILES: CC(C)Cn1c(=O)n(CC(O)CN2CCc3ccccc3C2)c(=O)c2ccncc21] | 41 |
| 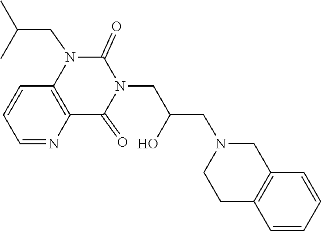 [SMILES: CC(C)Cn1c(=O)n(CC(O)CN2CCc3ccccc3C2)c(=O)c2ncccc21] | 42 |
| 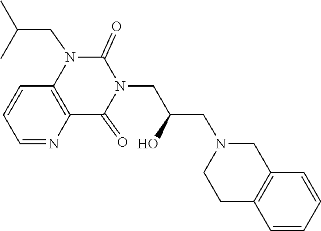 [SMILES: CC(C)Cn1c(=O)n(C[C@H](O)CN2CCc3ccccc3C2)c(=O)c2ncccc21] | 43 |
| 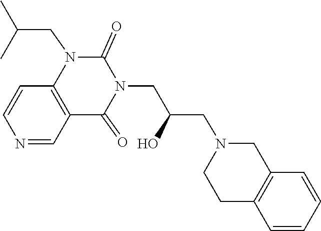 [SMILES: CC(C)Cn1c(=O)n(C[C@H](O)CN2CCc3ccccc3C2)c(=O)c2cnccc21] | 44 |
| 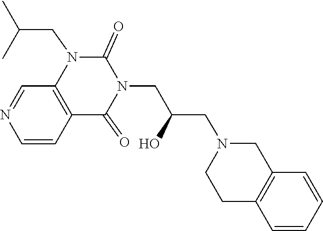 [SMILES: CC(C)Cn1c(=O)n(C[C@H](O)CN2CCc3ccccc3C2)c(=O)c2ccncc21] | 45 |
| 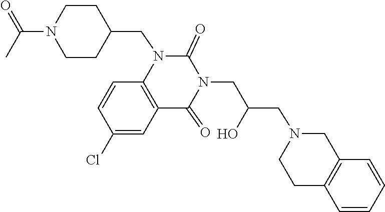 [SMILES: CC(=O)N1CCC(Cn2c(=O)n(CC(O)CN3CCc4ccccc4C3)c(=O)c3cc(Cl)ccc32)CC1] | 46 |
| 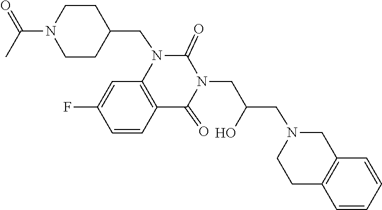 [SMILES: CC(=O)N1CCC(Cn2c(=O)n(CC(O)CN3CCc4ccccc4C3)c(=O)c3ccc(F)cc32)CC1] | 47 |
| 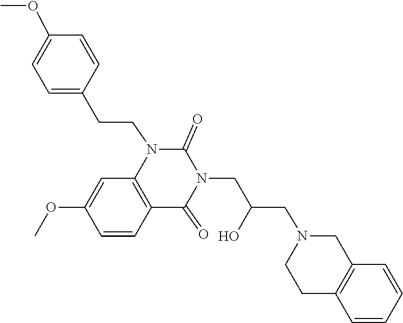 [SMILES: COc1ccc(CCn2c(=O)n(CC(O)CN3CCc4ccccc4C3)c(=O)c3ccc(OC)cc32)cc1] | 48 |
| 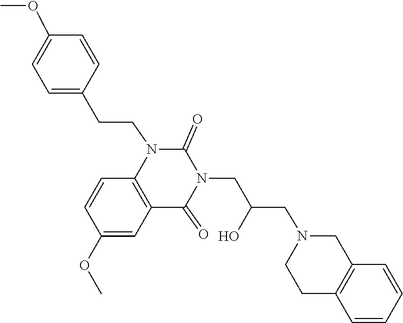 [SMILES: COc1ccc(CCn2c(=O)n(CC(O)CN3CCc4ccccc4C3)c(=O)c3cc(OC)ccc32)cc1] | 49 |
| 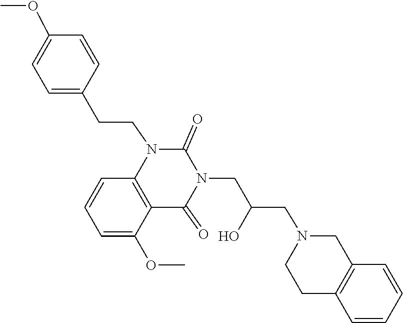 [SMILES: COc1ccc(CCn2c(=O)n(CC(O)CN3CCc4ccccc4C3)c(=O)c3c(OC)cccc32)cc1] | 50 |
| 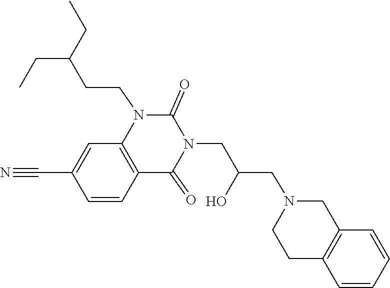 [SMILES: CCC(CC)CCn1c(=O)n(CC(O)CN2CCc3ccccc3C2)c(=O)c2ccc(C#N)cc21] | 51 |
| 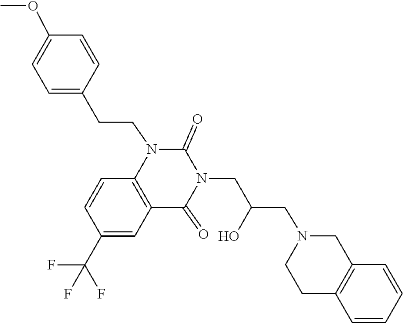 [SMILES: COc1ccc(CCn2c(=O)n(CC(O)CN3CCc4ccccc4C3)c(=O)c3cc(C(F)(F)F)ccc32)cc1] | 52 |
| 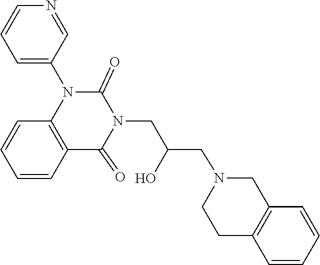 [SMILES: O=c1c2ccccc2n(-c2cccnc2)c(=O)n1CC(O)CN1CCc2ccccc2C1] | 53 |
| 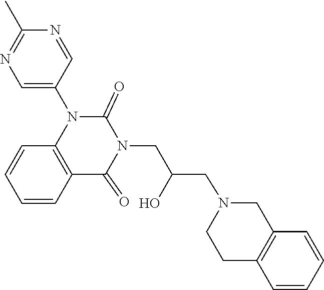 [SMILES: Cc1ncc(-n2c(=O)n(CC(O)CN3CCc4ccccc4C3)c(=O)c3ccccc32)cn1] | 54 |
| 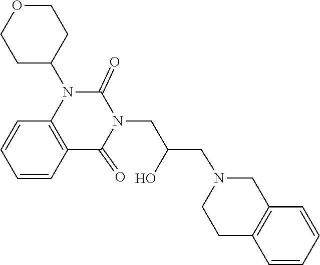 [SMILES: O=c1c2ccccc2n(C2CCOCC2)c(=O)n1CC(O)CN1CCc2ccccc2C1] | 55 |
| 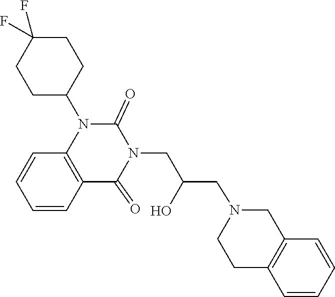 [SMILES: O=c1c2ccccc2n(C2CCC(F)(F)CC2)c(=O)n1CC(O)CN1CCc2ccccc2C1] | 56 |
| 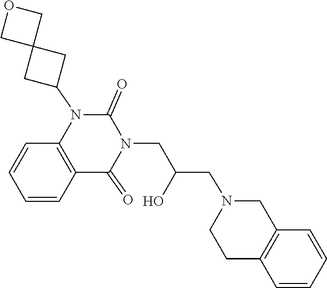 [SMILES: O=c1c2ccccc2n(C2CC3(COC3)C2)c(=O)n1CC(O)CN1CCc2ccccc2C1] | 57 |
| 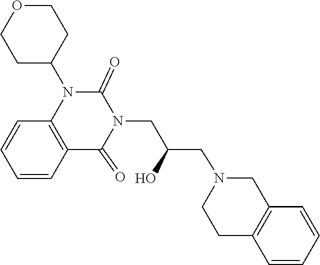 [SMILES: O=c1c2ccccc2n(C2CCOCC2)c(=O)n1C[C@H](O)CN1CCc2ccccc2C1] | 58 |
| 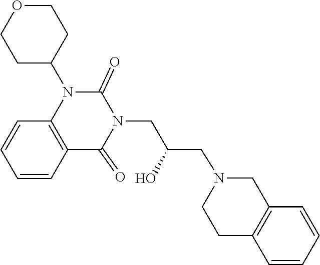 [SMILES: O=c1c2ccccc2n(C2CCOCC2)c(=O)n1C[C@@H](O)CN1CCc2ccccc2C1] | 59 |
| 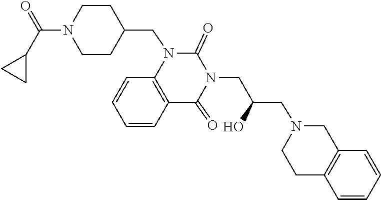 [SMILES: O=C(C1CC1)N1CCC(Cn2c(=O)n(C[C@H](O)CN3CCc4ccccc4C3)c(=O)c3ccccc32)CC1] | 60 |
| 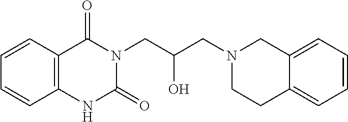 [SMILES: O=c1[nH]c2ccccc2c(=O)n1CC(O)CN1CCc2ccccc2C1] | 61 |
| 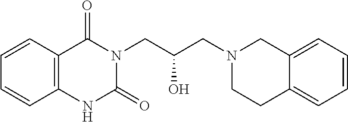 [SMILES: O=c1[nH]c2ccccc2c(=O)n1C[C@@H](O)CN1CCc2ccccc2C1] | 62 |
| 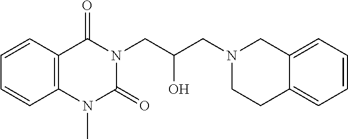 [SMILES: Cn1c(=O)n(CC(O)CN2CCc3ccccc3C2)c(=O)c2ccccc21] | 63 |
| 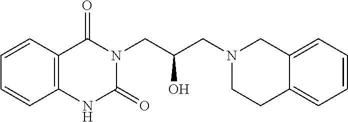 [SMILES: O=c1[nH]c2ccccc2c(=O)n1C[C@H](O)CN1CCc2ccccc2C1] | 64 |
| 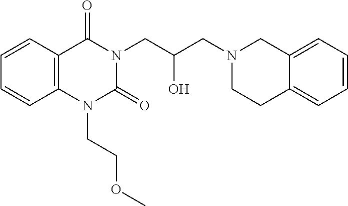 [SMILES: COCCn1c(=O)n(CC(O)CN2CCc3ccccc3C2)c(=O)c2ccccc21] | 65 |
| 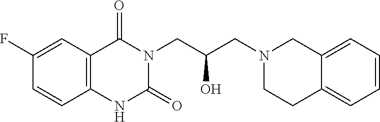 [SMILES: O=c1[nH]c2ccc(F)cc2c(=O)n1C[C@H](O)CN1CCc2ccccc2C1] | 66 |
| 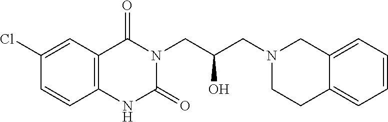 [SMILES: O=c1[nH]c2ccc(Cl)cc2c(=O)n1C[C@H](O)CN1CCc2ccccc2C1] | 67 |
| 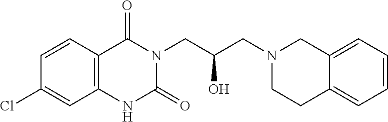 [SMILES: O=c1[nH]c2cc(Cl)ccc2c(=O)n1C[C@H](O)CN1CCc2ccccc2C1] | 68 |
| 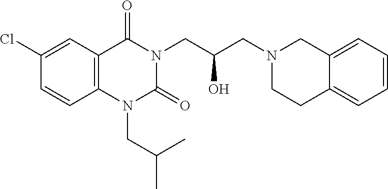 [SMILES: CC(C)Cn1c(=O)n(C[C@H](O)CN2CCc3ccccc3C2)c(=O)c2cc(Cl)ccc21] | 69 |
| 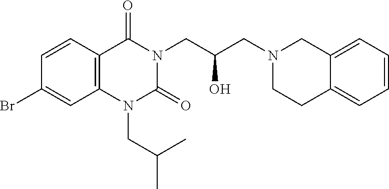 [SMILES: CC(C)Cn1c(=O)n(C[C@H](O)CN2CCc3ccccc3C2)c(=O)c2ccc(Br)cc21] | 70 |
| 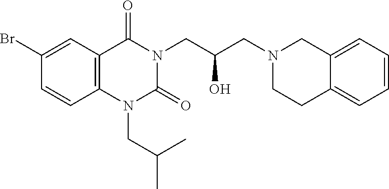 [SMILES: CC(C)Cn1c(=O)n(C[C@H](O)CN2CCc3ccccc3C2)c(=O)c2cc(Br)ccc21] | 71 |
| 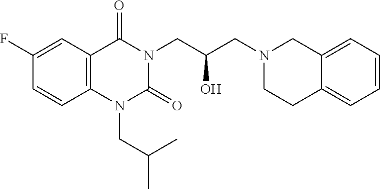 [SMILES: CC(C)Cn1c(=O)n(C[C@H](O)CN2CCc3ccccc3C2)c(=O)c2cc(F)ccc21] | 72 |
| 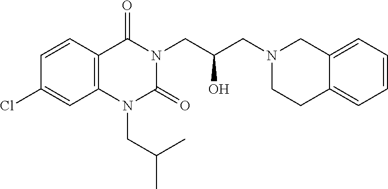 [SMILES: CC(C)Cn1c(=O)n(C[C@H](O)CN2CCc3ccccc3C2)c(=O)c2ccc(Cl)cc21] | 73 |
| 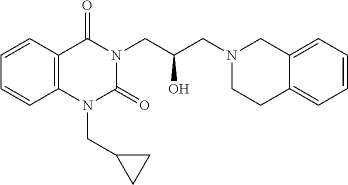 [SMILES: O=c1c2ccccc2n(CC2CC2)c(=O)n1C[C@H](O)CN1CCc2ccccc2C1] | 74 |
| 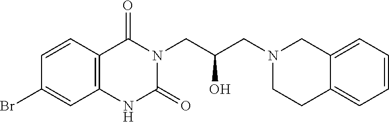 [SMILES: O=c1[nH]c2cc(Br)ccc2c(=O)n1C[C@H](O)CN1CCc2ccccc2C1] | 75 |
| 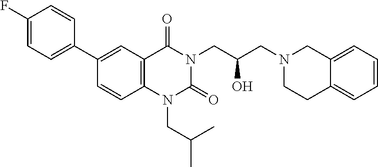 [SMILES: CC(C)Cn1c(=O)n(C[C@H](O)CN2CCc3ccccc3C2)c(=O)c2cc(-c3ccc(F)cc3)ccc21] | 76 |
| 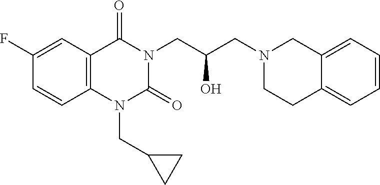 [SMILES: O=c1c2cc(F)ccc2n(CC2CC2)c(=O)n1C[C@H](O)CN1CCc2ccccc2C1] | 77 |
| 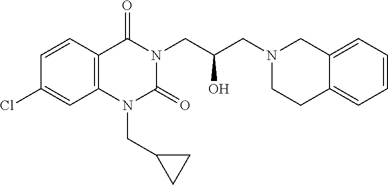 [SMILES: O=c1c2ccc(Cl)cc2n(CC2CC2)c(=O)n1C[C@H](O)CN1CCc2ccccc2C1] | 78 |
| 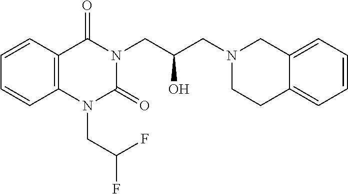 [SMILES: O=c1c2ccccc2n(CC(F)F)c(=O)n1C[C@H](O)CN1CCc2ccccc2C1] | 79 |
| 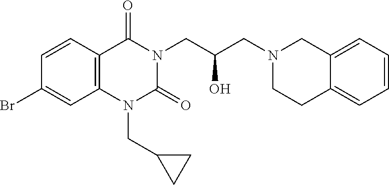 [SMILES: O=c1c2ccc(Br)cc2n(CC2CC2)c(=O)n1C[C@H](O)CN1CCc2ccccc2C1] | 80 |
| 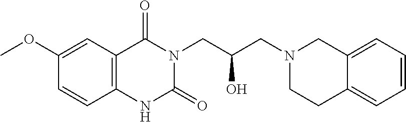 [SMILES: COc1ccc2[nH]c(=O)n(C[C@H](O)CN3CCc4ccccc4C3)c(=O)c2c1] | 81 |
| 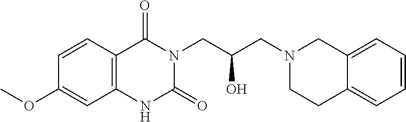 [SMILES: COc1ccc2c(=O)n(C[C@H](O)CN3CCc4ccccc4C3)c(=O)[nH]c2c1] | 82 |
| 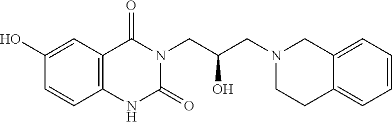 [SMILES: O=c1[nH]c2ccc(O)cc2c(=O)n1C[C@H](O)CN1CCc2ccccc2C1] | 83 |
| 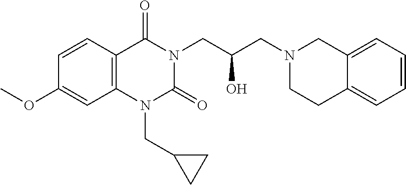 [SMILES: COc1ccc2c(=O)n(C[C@H](O)CN3CCc4ccccc4C3)c(=O)n(CC3CC3)c2c1] | 84 |
| 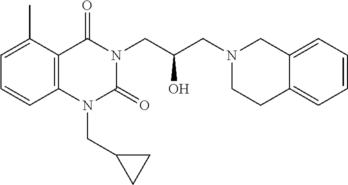 [SMILES: Cc1cccc2c1c(=O)n(C[C@H](O)CN1CCc3ccccc3C1)c(=O)n2CC1CC1] | 85 |
| 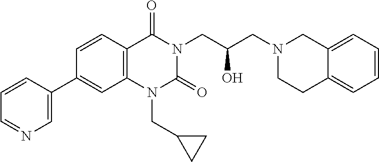 [SMILES: O=c1c2ccc(-c3cccnc3)cc2n(CC2CC2)c(=O)n1C[C@H](O)CN1CCc2ccccc2C1] | 86 |
| 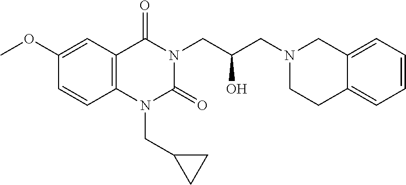 [SMILES: COc1ccc2c(c1)c(=O)n(C[C@H](O)CN1CCc3ccccc3C1)c(=O)n2CC1CC1] | 87 |
| 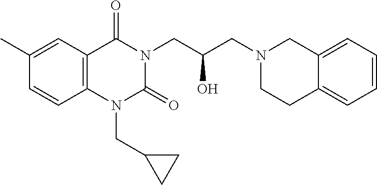 [SMILES: Cc1ccc2c(c1)c(=O)n(C[C@H](O)CN1CCc3ccccc3C1)c(=O)n2CC1CC1] | 88 |
| 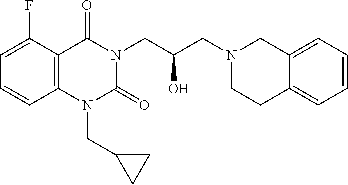 [SMILES: O=c1c2c(F)cccc2n(CC2CC2)c(=O)n1C[C@H](O)CN1CCc2ccccc2C1] | 89 |
| 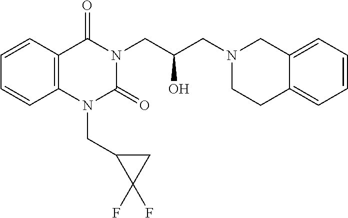 [SMILES: O=c1c2ccccc2n(CC2CC2(F)F)c(=O)n1C[C@H](O)CN1CCc2ccccc2C1] | 90 |
| 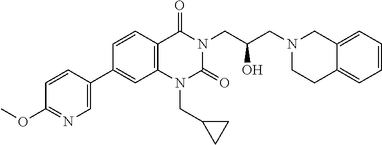 [SMILES: COc1ccc(-c2ccc3c(=O)n(C[C@H](O)CN4CCc5ccccc5C4)c(=O)n(CC4CC4)c3c2)cn1] | 91 |
| 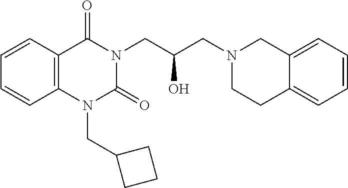 [SMILES: O=c1c2ccccc2n(CC2CCC2)c(=O)n1C[C@H](O)CN1CCc2ccccc2C1] | 92 |
| 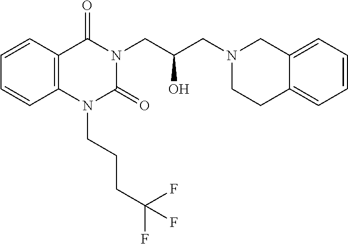 [SMILES: O=c1c2ccccc2n(CCCC(F)(F)F)c(=O)n1C[C@H](O)CN1CCc2ccccc2C1] | 93 |
| 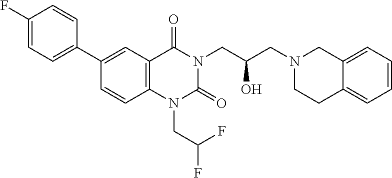 [SMILES: O=c1c2cc(-c3ccc(F)cc3)ccc2n(CC(F)F)c(=O)n1C[C@H](O)CN1CCc2ccccc2C1] | 94 |
| 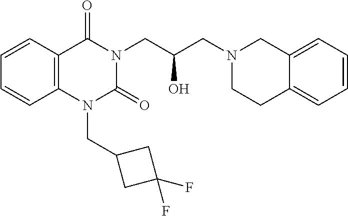 [SMILES: O=c1c2ccccc2n(CC2CC(F)(F)C2)c(=O)n1C[C@H](O)CN1CCc2ccccc2C1] | 95 |
| 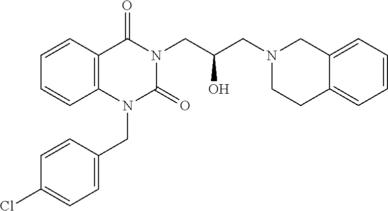 [SMILES: O=c1c2ccccc2n(Cc2ccc(Cl)cc2)c(=O)n1C[C@H](O)CN1CCc2ccccc2C1] | 96 |
| 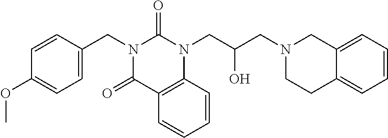 [SMILES: COc1ccc(Cn2c(=O)c3ccccc3n(CC(O)CN3CCc4ccccc4C3)c2=O)cc1] | 97 |
| 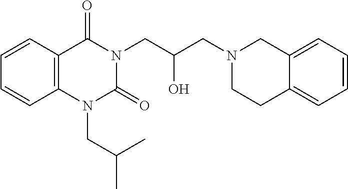 [SMILES: CC(C)Cn1c(=O)n(CC(O)CN2CCc3ccccc3C2)c(=O)c2ccccc21] | 98 |
| 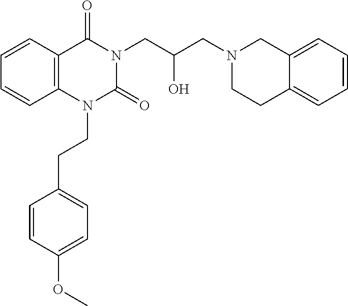 [SMILES: COc1ccc(CCn2c(=O)n(CC(O)CN3CCc4ccccc4C3)c(=O)c3ccccc32)cc1] | 99 |
| 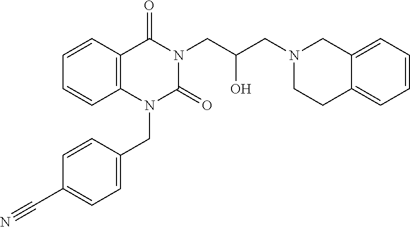 [SMILES: N#Cc1ccc(Cn2c(=O)n(CC(O)CN3CCc4ccccc4C3)c(=O)c3ccccc32)cc1] | 100 |
| 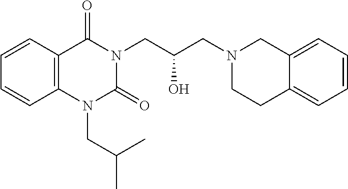 [SMILES: CC(C)Cn1c(=O)n(C[C@@H](O)CN2CCc3ccccc3C2)c(=O)c2ccccc21] | 101 |
| 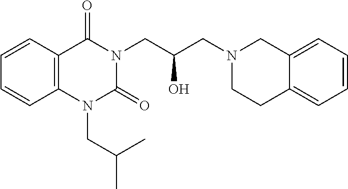 [SMILES: CC(C)Cn1c(=O)n(C[C@H](O)CN2CCc3ccccc3C2)c(=O)c2ccccc21] | 102 |
| 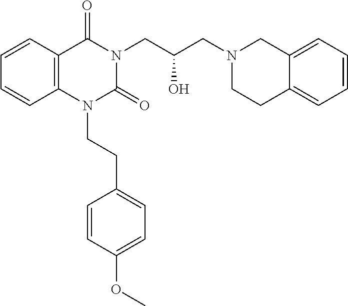 [SMILES: COc1ccc(CCn2c(=O)n(C[C@@H](O)CN3CCc4ccccc4C3)c(=O)c3ccccc32)cc1] | 103 |
| 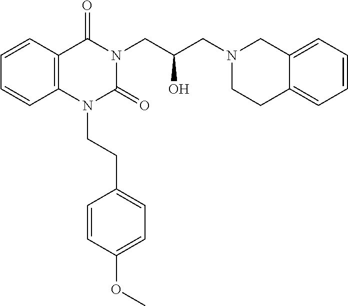 [SMILES: COc1ccc(CCn2c(=O)n(C[C@H](O)CN3CCc4ccccc4C3)c(=O)c3ccccc32)cc1] | 104 |
| 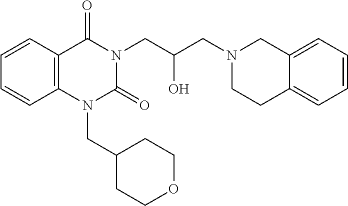 [SMILES: O=c1c2ccccc2n(CC2CCOCC2)c(=O)n1CC(O)CN1CCc2ccccc2C1] | 105 |

TABLE 2
IUPAC names of compounds in Table 1.
IUPAC names (these names are provided in the same
cell order to correspond to the structures in Table 1):

| | |
|---|---|
| 1-[(4-chlorophenyl)methyl]-3-[3-(3,4- | 3-[3-(3,4-dihydro-1H-isoquinolin-2-yl)-2- |
| dihydro-1H-isoquinolin-2-yl)-2-hydroxy- | hydroxy-propyl]-1-isobutyl-quinazoline- |
| propyl]quinazoline-2,4-dione | 2,4-dione |
| 3-[3-(3,4-dihydro-1H-isoquinolin-2-yl)-2- | 3-[3-(3,4-dihydro-1H-isoquinolin-2-yl)-2- |
| hydroxy-propyl]-1-[2-(4- | hydroxy-propyl]-1-[(4- |
| methoxyphenyl)ethyl]quinazoline-2,4- | methoxyphenyl)methyl]quinazoline-2,4- |
| dione | dione |
| 3-[(2R)-3-(3,4-dihydro-1H-isoquinolin-2- | 3-[(2S)-3-(3,4-dihydro-1H-isoquinolin-2- |
| yl)-2-hydroxy-propyl]-1-[2-(4- | yl)-2-hydroxy-propyl]-1-[2-(4- |
| methoxyphenyl)ethyl]quinazoline-2,4- | methoxyphenyl)ethyl]quinazoline-2,4- |
| dione | dione |
| 3-[3-(3,4-dihydro-1H-isoquinolin-2-yl)-2- | 3-[(2R)-3-(3,4-dihydro-1H-isoquinolin-2- |
| hydroxy-propyl]-1-(4- | yl)-2-hydroxy-propyl]-1-isobutyl- |
| methoxyphenyl)quinazoline-2,4-dione | quinazoline-2,4-dione |
| 3-[(2S)-3-(3,4-dihydro-1H-isoquinolin-2- | 3-[3-(3,4-dihydro-1H-isoquinolin-2-yl)-2- |
| yl)-2-hydroxy-propyl]-1-isobutyl- | hydroxy-propyl]-1-[1-(4- |
| quinazoline-2,4-dione | methoxyphenyl)ethyl]quinazoline-2,4-dione |
| 3-[3-(3,4-dihydro-1H-isoquinolin-2-yl)-2- | 3-[3-(3,4-dihydro-1H-isoquinolin-2-yl)-2- |
| hydroxy-propyl]-1-(2- | hydroxy-propyl]-1-[(3- |
| morpholinoethyl)quinazoline-2,4-dione | methoxyphenyl)methyl]quinazoline-2,4-dione |
| 3-[3-(3,4-dihydro-1H-isoquinolin-2-yl)-2- | 4-[[3-[3-(3,4-dihydro-1H-isoquinolin-2-yl)- |
| hydroxy-propyl]-1-[(4- | 2-hydroxy-propyl]-2,4-dioxo-quinazolin-1- |
| hydroxyphenyl)methyl]quinazoline-2,4-dione | yl]methyl]benzonitrile |
| 4-[[3-[(2R)-3-(3,4-dihydro-1H-isoquinolin- | 4-[[3-[(2S)-3-(3,4-dihydro-1H-isoquinolin- |
| 2-yl)-2-hydroxy-propyl]-2,4-dioxo- | 2-yl)-2-hydroxy-propyl]-2,4-dioxo- |
| quinazolin-1-yl]methyl]benzonitrile | quinazolin-1-yl]methyl]benzonitrile |
| 1-[2-(3,5-dichlorophenyl)ethyl]-3-[3-(3,4- | 3-[3-(3,4-dihydro-1H-isoquinolin-2-yl)-2- |
| dihydro-1H-isoquinolin-2-yl)-2-hydroxy- | hydroxy-propyl]-1-(tetrahydropyran-4- |
| propyl]quinazoline-2,4-dione | ylmethyl)quinazoline-2,4-dione |
| 1-[(1-acetyl-4-piperidyl)methyl]-3-[3-(3,4- | 4-[3-[3-(3,4-dihydro-1H-isoquinolin-2-yl)- |
| dihydro-1H-isoquinolin-2-yl)-2-hydroxy- | 2-hydroxy-propyl]-2,4-dioxo-quinazolin-1- |
| propyl]quinazoline-2,4-dione | yl]benzonitrile |
| 3-[3-(3,4-dihydro-1H-isoquinolin-2-yl)-2- | 3-[2-[3-[3-(3,4-dihydro-1H-isoquinolin-2- |
| hydroxy-propyl]-1-isohexyl-quinazoline- | yl)-2-hydroxy-propyl]-2,4-dioxo- |
| 2,4-dione | quinazolin-1-yl]ethyl]benzonitrile |
| 3-[3-(3,4-dihydro-1H-isoquinolin-2-yl)-2- | 3-[3-(3,4-dihydro-1H-isoquinolin-2-yl)-2- |
| hydroxy-propyl]-1-(3- | hydroxy-propyl]-1-[(6-methoxy-3- |
| pyridylmethyl)quinazoline-2,4-dione | pyridyl)methyl]quinazoline-2,4-dione |
| 3-[3-(3,4-dihydro-1H-isoquinolin-2-yl)-2- | 3-[3-(3,4-dihydro-1H-isoquinolin-2-yl)-2- |
| hydroxy-propyl]-1-[(2-methylpyrimidin-5- | hydroxy-propyl]-1-[2-(3- |
| yl)methyl]quinazoline-2,4-dione | pyridyl)ethyl]quinazoline-2,4-dione |
| 3-[3-(3,4-dihydro-1H-isoquinolin-2-yl)-2- | 4-[3-[3-(3,4-dihydro-1H-isoquinolin-2-yl)- |
| hydroxy-propyl]-1-[(1-ethyl-6-oxo-3- | 2-hydroxy-propyl]-2,4-dioxo-quinazolin-1- |
| pyridyl)methyl]quinazoline-2,4-dione | yl]-N-methyl-butanamide |
| 4-[3-[3-(3,4-dihydro-1H-isoquinolin-2-yl)- | 3-[3-(3,4-dihydro-1H-isoquinolin-2-yl)-2- |
| 2-hydroxy-propyl]-2,4-dioxo-quinazolin-1- | hydroxy-propyl]-1-(4-oxo-4-pyrrolidin-1-yl- |
| yl]-N,N-dimethyl-butanamide | butyl)quinazoline-2,4-dione |
| 3-[3-(3,4-dihydro-1H-isoquinolin-2-yl)-2- | 3-[3-(3,4-dihydro-1H-isoquinolin-2-yl)-2- |
| hydroxy-propyl]-1-(4-morpholino-4-oxo- | hydroxy-propyl]-1-[2-(2-oxopyrrolidin-1- |
| butyl)quinazoline-2,4-dione | yl)ethyl]quinazoline-2,4-dione |
| 3-[3-(3,4-dihydro-1H-isoquinolin-2-yl)-2- | 3-[3-(3,4-dihydro-1H-isoquinolin-2-yl)-2- |
| hydroxy-propyl]-1-(2- | hydroxy-propyl]-1-(2-tetrahydropyran-4- |
| ethylbutyl)quinazoline-2,4-dione | yloxyethyl)quinazoline-2,4-dione |
| 3-[3-(3,4-dihydro-1H-isoquinolin-2-yl)-2- | 3-[3-(3,4-dihydro-1H-isoquinolin-2-yl)-2- |
| hydroxy-propyl]-1-[2-(2-oxa-8- | hydroxy-propyl]-1-[2-(2-oxa-7- |
| azaspiro[4.5]decan-8-yl)ethyl]quinazoline- | azaspiro[4.4]nonan-7-yl)ethyl]quinazoline- |
| 2,4-dione | 2,4-dione |
| 3-[3-(3,4-dihydro-1H-isoquinolin-2-yl)-2- | 3-[3-(3,4-dihydro-1H-isoquinolin-2-yl)-2- |
| hydroxy-propyl]-1-[2-(2-oxa-7- | hydroxy-propyl]-1-[2-(3-methoxyazetidin- |
| azaspiro[4.4]nonan-7-yl)-2-oxo- | 1-yl)ethyl]quinazoline-2,4-dione |
| ethyl]quinazoline-2,4-dione | |
| 1-[2-(2,3-dihydro-1,4-benzodioxin-6- | 3-[3-(3,4-dihydro-1H-isoquinolin-2-yl)-2- |
| yl)ethyl]-3-[3-(3,4-dihydro-1H-isoquinolin- | hydroxy-propyl]-1-isobutyl-pyrido[4,3- |
| 2-yl)-2-hydroxy-propyl]quinazoline-2,4- | d]pyrimidine-2,4-dione |
| dione | |
| 3-[3-(3,4-dihydro-1H-isoquinolin-2-yl)-2- | 3-[3-(3,4-dihydro-1H-isoquinolin-2-yl)-2- |
| hydroxy-propyl]-1-isobutyl-pyrido[3,4- | hydroxy-propyl]-1-isobutyl-pyrido[3,2- |
| d]pyrimidine-2,4-dione | d]pyrimidine-2,4-dione |
| 3-[(2R)-3-(3,4-dihydro-1H-isoquinolin-2- | 3-[(2R)-3-(3,4-dihydro-1H-isoquinolin-2- |
| yl)-2-hydroxy-propyl]-1-isobutyl- | yl)-2-hydroxy-propyl]-1-isobutyl- |
| pyrido[3,2-d]pyrimidine-2,4-dione | pyrido[4,3-d]pyrimidine-2,4-dione |
| 3-[(2R)-3-(3,4-dihydro-1H-isoquinolin-2- | 1-[(1-acetyl-4-piperidyl)methyl]-6-chloro- |
| yl)-2-hydroxy-propyl]-1-isobutyl- | 3-[3-(3,4-dihydro-1H-isoquinolin-2-yl)-2- |
| pyrido[3,4-d]pyrimidine-2,4-dione | hydroxy-propyl]quinazoline-2,4-dione |
| 1-[(1-acetyl-4-piperidyl)methyl]-3-[3-(3,4- | 3-[3-(3,4-dihydro-1H-isoquinolin-2-yl)-2- |
| dihydro-1H-isoquinolin-2-yl)-2-hydroxy- | hydroxy-propyl]-7-methoxy-1-[2-(4- |
| propyl]-7-fluoro-quinazoline-2,4-dione | methoxyphenyl)ethyl]quinazoline-2,4-dione |
| 3-[3-(3,4-dihydro-1H-isoquinolin-2-yl)-2- | 3-[3-(3,4-dihydro-1H-isoquinolin-2-yl)-2- |
| hydroxy-propyl]-6-methoxy-1-[2-(4- | hydroxy-propyl]-5-methoxy-1-[2-(4- |
| methoxyphenyl)ethyl]quinazoline-2,4- | methoxyphenyl)ethyl]quinazoline-2,4- |
| dione | dione |
| 3-[3-(3,4-dihydro-1H-isoquinolin-2-yl)-2- | 3-[3-(3,4-dihydro-1H-isoquinolin-2-yl)-2- |
| hydroxy-propyl]-1-(3-ethylpentyl)-2,4- | hydroxy-propyl]-1-[2-(4- |
| dioxo-quinazoline-7-carbonitrile | methoxyphenyl)ethyl]-6- |
| | (trifluoromethyl)quinazoline-2,4-dione |
| 3-[3-(3,4-dihydro-1H-isoquinolin-2-yl)-2- | 3-[3-(3,4-dihydro-1H-isoquinolin-2-yl)-2- |
| hydroxy-propyl]-1-(3-pyridyl)quinazoline- | hydroxy-propyl]-1-(2-methylpyrimidin-5- |
| 2,4-dione | yl)quinazoline-2,4-dione |
| 3-[3-(3,4-dihydro-1H-isoquinolin-2-yl)-2- | 1-(4,4-difluorocyclohexyl)-3-[3-(3,4- |
| hydroxy-propyl]-1-tetrahydropyran-4-yl- | dihydro-1H-isoquinolin-2-yl)-2-hydroxy- |
| quinazoline-2,4-dione | propyl]quinazoline-2,4-dione |
| 3-[3-(3,4-dihydro-1H-isoquinolin-2-yl)-2- | 3-[(2R)-3-(3,4-dihydro-1H-isoquinolin-2- |
| hydroxy-propyl]-1-(2-oxaspiro[3.3]heptan- | yl)-2-hydroxy-propyl]-1-tetrahydropyran-4- |
| 6-yl)quinazoline-2,4-dione | yl-quinazoline-2,4-dione |
| 3-[(2S)-3-(3,4-dihydro-1H-isoquinolin-2- | 1-[[1-(cyclopropanecarbonyl)-4- |
| yl)-2-hydroxy-propyl]-1-tetrahydropyran-4- | piperidyl]methyl]-3-[(2R)-3-(3,4-dihydro- |
| yl-quinazoline-2,4-dione | 1H-isoquinolin-2-yl)-2-hydroxy- |
| | propyl]quinazoline-2,4-dione |
| 3-[3-(3,4-dihydro-1H-isoquinolin-2-yl)-2- | 3-[(2S)-3-(3,4-dihydro-1H-isoquinolin-2- |
| hydroxy-propyl]-1H-quinazoline-2,4-dione | yl)-2-hydroxy-propyl]-1H-quinazoline-2,4- |
| | dione |
| 3-[3-(3,4-dihydro-1H-isoquinolin-2-yl)-2- | 3-[(2R)-3-(3,4-dihydro-1H-isoquinolin-2- |
| hydroxy-propyl]-1-methyl-quinazoline-2,4- | yl)-2-hydroxy-propyl]-1H-quinazoline-2,4- |
| dione | dione |
| 3-[3-(3,4-dihydro-1H-isoquinolin-2-yl)-2- | 3-[(2R)-3-(3,4-dihydro-1H-isoquinolin-2- |
| hydroxy-propyl]-1-(2- | yl)-2-hydroxy-propyl]-6-fluoro-1H- |
| methoxyethyl)quinazoline-2,4-dione | quinazoline-2,4-dione |
| 6-chloro-3-[(2R)-3-(3,4-dihydro-1H- | 7-chloro-3-[(2R)-3-(3,4-dihydro-1H- |
| isoquinolin-2-yl)-2-hydroxy-propyl]-1H- | isoquinolin-2-yl)-2-hydroxy-propyl]-1H- |
| quinazoline-2,4-dione | quinazoline-2,4-dione |
| 6-chloro-3-[(2R)-3-(3,4-dihydro-1H- | 7-bromo-3-[(2R)-3-(3,4-dihydro-1H- |
| isoquinolin-2-yl)-2-hydroxy-propyl]-1- | isoquinolin-2-yl)-2-hydroxy-propyl]-1- |
| isobutyl-quinazoline-2,4-dione | isobutyl-quinazoline-2,4-dione |
| 6-bromo-3-[(2R)-3-(3,4-dihydro-1H- | 3-[(2R)-3-(3,4-dihydro-1H-isoquinolin-2- |
| isoquinolin-2-yl)-2-hydroxy-propyl]-1- | yl)-2-hydroxy-propyl]-6-fluoro-1-isobutyl- |
| isobutyl-quinazoline-2,4-dione | quinazoline-2,4-dione |
| 7-chloro-3-[(2R)-3-(3,4-dihydro-1H- | 1-(cyclopropylmethyl)-3-[(2R)-3-(3,4- |
| isoquinolin-2-yl)-2-hydroxy-propyl]-1- | dihydro-1H-isoquinolin-2-yl)-2-hydroxy- |
| isobutyl-quinazoline-2,4-dione | propyl]quinazoline-2,4-dione |
| 7-bromo-3-[(2R)-3-(3,4-dihydro-1H- | 3-[(2R)-3-(3,4-dihydro-1H-isoquinolin-2- |
| isoquinolin-2-yl)-2-hydroxy-propyl]-1H- | yl)-2-hydroxy-propyl]-6-(4-fluorophenyl)-1- |
| quinazoline-2,4-dione | isobutyl-quinazoline-2,4-dione |
| 1-(cyclopropylmethyl)-3-[(2R)-3-(3,4- | 7-chloro-1-(cyclopropylmethyl)-3-[(2R)-3- |
| dihydro-1H-isoquinolin-2-yl)-2-hydroxy- | (3,4-dihydro-1H-isoquinolin-2-yl)-2- |
| propyl]-6-fluoro-quinazoline-2,4-dione | hydroxy-propyl]quinazoline-2,4-dione |
| 1-(2,2-difluoroethyl)-3-[(2R)-3-(3,4- | 7-bromo-1-(cyclopropylmethyl)-3-[(2R)-3- |
| dihydro-1H-isoquinolin-2-yl)-2-hydroxy- | (3,4-dihydro-1H-isoquinolin-2-yl)-2- |
| propyl]quinazoline-2,4-dione | hydroxy-propyl]quinazoline-2,4-dione |
| 3-[(2R)-3-(3,4-dihydro-1H-isoquinolin-2- | 3-[(2R)-3-(3,4-dihydro-1H-isoquinolin-2- |
| yl)-2-hydroxy-propyl]-6-methoxy-1H- | yl)-2-hydroxy-propyl]-7-methoxy-1H- |
| quinazoline-2,4-dione | quinazoline-2,4-dione |
| 3-[(2R)-3-(3,4-dihydro-1H-isoquinolin-2- | 1-(cyclopropylmethyl)-3-[(2R)-3-(3,4- |
| yl)-2-hydroxy-propyl]-6-hydroxy-1H- | dihydro-1H-isoquinolin-2-yl)-2-hydroxy- |
| quinazoline-2,4-dione | propyl]-7-methoxy-quinazoline-2,4-dione |
| 1-(cyclopropylmethyl)-3-[(2R)-3-(3,4- | 1-(cyclopropylmethyl)-3-[(2R)-3-(3,4- |
| dihydro-1H-isoquinolin-2-yl)-2-hydroxy- | dihydro-1H-isoquinolin-2-yl)-2-hydroxy- |
| propyl]-5-methyl-quinazoline-2,4-dione | propyl]-7-(3-pyridyl)quinazoline-2,4-dione |
| 1-(cyclopropylmethyl)-3-[(2R)-3-(3,4- | 1-(cyclopropylmethyl)-3-[(2R)-3-(3,4- |
| dihydro-1H-isoquinolin-2-yl)-2-hydroxy- | dihydro-1H-isoquinolin-2-yl)-2-hydroxy- |
| propyl]-6-methoxy-quinazoline-2,4-dione | propyl]-6-methyl-quinazoline-2,4-dione |
| 1-(cyclopropylmethyl)-3-[(2R)-3-(3,4- | 1-[(2,2-difluorocyclopropyl)methyl]-3- |
| dihydro-1H-isoquinolin-2-yl)-2-hydroxy- | [(2R)-3-(3,4-dihydro-1H-isoquinolin-2-yl)- |
| propyl]-5-fluoro-quinazoline-2,4-dione | 2-hydroxy-propyl]quinazoline-2,4-dione |
| 1-(cyclopropylmethyl)-3-[(2R)-3-(3,4- | 1-(cyclobutylmethyl)-3-[(2R)-3-(3,4- |
| dihydro-1H-isoquinolin-2-yl)-2-hydroxy- | dihydro-1H-isoquinolin-2-yl)-2-hydroxy- |
| propyl]-7-(6-methoxy-3- | propyl]quinazoline-2,4-dione |
| pyridyl)quinazoline-2,4-dione | |
| 3-[(2R)-3-(3,4-dihydro-1H-isoquinolin-2- | 1-(2,2-difluoroethyl)-3-[(2R)-3-(3,4- |
| yl)-2-hydroxy-propyl]-1-(4,4,4- | dihydro-1H-isoquinolin-2-yl)-2-hydroxy-propyl]- |
| trifluorobutyl)quinazoline-2,4-dione | 6-(4-fluorophenyl)quinazoline-2,4-dione |
| 1-[(3,3-difluorocyclobutyl)methyl]-3-[(2R)- | 1-[(4-chlorophenyl)methyl]-3-[3-(3,4- |
| 3-(3,4-dihydro-1H-isoquinolin-2-yl)-2- | dihydro-1H-isoquinolin-2-yl)-2-hydroxy- |
| hydroxy-propyl]quinazoline-2,4-dione | propyl]quinazoline-2,4-dione |
| 1-[3-(3,4-dihydro-1H-isoquinolin-2-yl)-2- | 3-[3-(3,4-dihydro-1H-isoquinolin-2-yl)-2- |
| hydroxy-propyl]-3-[(4- | hydroxy-propyl]-1-isobutyl-quinazoline- |
| methoxyphenyl)methyl]quinazoline-2,4-dione | 2,4-dione |
| 3-[3-(3,4-dihydro-1H-isoquinolin-2-yl)-2- | 4-[[3-[3-(3,4-dihydro-1H-isoquinolin-2-yl)- |
| hydroxy-propyl]-1-[2-(4- | 2-hydroxy-propyl]-2,4-dioxo-quinazolin-1- |
| methoxyphenyl)ethyl]quinazoline-2,4-dione | yl]methyl]benzonitrile |
| 3-[(2S)-3-(3,4-dihydro-1H-isoquinolin-2- | 3-[(2R)-3-(3,4-dihydro-1H-isoquinolin-2- |
| yl)-2-hydroxy-propyl]-1-isobutyl- | yl)-2-hydroxy-propyl]-1-isobutyl- |
| quinazoline-2,4-dione | quinazoline-2,4-dione |
| 3-[(2S)-3-(3,4-dihydro-1H-isoquinolin-2- | 3-[(2R)-3-(3,4-dihydro-1H-isoquinolin-2- |
| yl)-2-hydroxy-propyl]-1-[2-(4- | yl)-2-hydroxy-propyl]-1-[2-(4- |
| methoxyphenyl)ethyl]quinazoline-2,4-dione | methoxyphenyl)ethyl]quinazoline-2,4-dione |
| 3-[3-(3,4-dihydro-1H-isoquinolin-2-yl)-2- | |
| hydroxy-propyl]-1-(tetrahydropyran-4- | |
| ylmethyl)quinazoline-2,4-dione | |

### Methods of Treatment

The compounds of the present invention are useful for treating disease states such as cancers.

Without being limited by theory, the compounds of the present invention are believed to act as inhibitors of PRMT-5.

For cancer therapy, the compounds of the present invention can be useful for treating cancers. In non-limiting embodiments, the compounds of the present invention are useful for treating cancers including solid tumors such as myelodysplastic syndrome (MDS), acute myeloid leukemia (AML), mantle cell lymphoma (MCL) and non-Hodgkin's lymphoma (NHL); Melanoma and breast cancer.

### Definitions

The term “heteroatom(s)” as used herein means an atom selected from nitrogen, which can be quaternized or present as an oxide; oxygen; and sulfur, including oxidized sulfurs including, sulfoxide and sulfone, and in some cases sulfonate. In certain instances, the compounds and/or synthetic intermediates may include other heteroatoms such as boron, phosphorous, and silicon.

As an example, the term “C₁-C₆” preceding another term such as “alkyl” or “carbocycle” or any other chemical group indicates a number of carbons in said group. For example, a C₁-C₆ alkyl will contain between 1-6 carbon atoms under the definition of alkyl which follows. If the group contains heteroatoms (such as a heteroalkyl) it can be appreciated that a C₁-C₆ heteroalkyl, heterocycle, etc. will contain between 1-6 atoms in the group, including carbon and heteroatoms.

The term “alkyl” as used herein encompasses saturated alkyl as well as unsaturated alkyl such as alkenyl, alkynyl, and the like. The term “alkyl” as used herein means normal, secondary, or tertiary, linear or branched hydrocarbon with no site of unsaturation. Examples are methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. The term “alkenyl” as used herein means normal, secondary or tertiary, linear or branched hydrocarbon with at least one site (usually 1 to 3, preferably 1) of unsaturation, namely a carbon-carbon, sp2 double bond. Examples include, but are not limited to: ethylene or vinyl (—CH=CH₂), allyl (—CH₂CH═CH₂), and 5-hexenyl (—CH₂CH₂CH₂CH₂CH═CH₂). The double bond may be in the cis or trans configuration. The term “alkynyl” as used herein means normal, secondary, tertiary, linear or branched hydrocarbon with at least one site (usually 1 to 3, preferably 1) of unsaturation, namely a carbon-carbon, sp triple bond. Examples include, but are not limited to: ethynyl (—C═CH), and 1-propynyl (propargyl, —CH₂C═CH). Unless specified otherwise, an “alkyl” may include between 1-10 carbon atoms, or in some cases preferably 1-6 carbon atoms.

The term “alkylene” as used herein encompasses saturated alkylene as well as unsaturated alkylene such as alkenylene, alkynylene, alkenynylene and the like. The term “alkylene” as used herein means saturated, linear or branched chain hydrocarbon radical having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like. The term “alkenylene” as used herein means linear or branched chain hydrocarbon radical with at least one site (usually 1 to 3, preferably 1) of unsaturation, namely a carbon-carbon, sp2 double bond, and having two monovalent radical centers derived by the removal of two hydrogen atoms from the same or two different carbon atoms of a parent alkene. The term “alkynylene” as used herein means linear or branched chain hydrocarbon radical with at least one site (usually 1 to 3, preferably 1) of unsaturation, namely a carbon-carbon, sp triple bond, and having two monovalent radical centers derived by the removal of two hydrogen atoms from the same or two different carbon atoms of a parent alkyne. Unless specified otherwise, an “alkylene” may include between 1-10 carbon atoms, or in some cases preferably 1-6 carbon atoms.

The term “heteroalkyl” as used herein encompasses saturated heteroalkyl as well as unsaturated heteroalkyl such as heteroalkenyl, heteroalkynyl, heteroalkenynyl and the like. The term “heteroalkyl” as used herein means linear or branched chain alkyl wherein one or more carbon atoms (usually 1, 2 or 3) are replaced by a heteroatom, i.e., an oxygen, nitrogen or sulfur atom, with the proviso that said chain may not contain two adjacent O atoms or two adjacent S atoms. This means that one or more —CH₃ of said alkyl can be replaced by —NH₂ and/or that one or more —CH₂— of said alkyl can be replaced by —NH—, —O— or —S—. The S atoms in said chains may be optionally oxidized with one or two oxygen atoms, to afford sulfoxides and sulfones, respectively. Furthermore, heteroalkyl groups can contain an oxo or thio group at any carbon or heteroatom that will result in a stable compound. Exemplary heteroalkyl groups include, but are not limited to, alcohols, alkyl ethers (such as for example -methoxy, -ethoxy, -butoxy . . . ), primary, secondary, and tertiary alkyl amines, amides, ketones, esters, alkyl sulfides, and alkyl sulfones. The term “heteroalkenyl” means linear or branched chain alkenyl wherein one or more carbon atoms (usually 1, 2 or 3) are replaced by an oxygen, nitrogen or sulfur atom, with the proviso that said chain may not contain two adjacent O atoms or two adjacent S atoms. The term heteroalkenyl thus comprises imines, —O-alkenyl, —NH-alkenyl, —N(alkenyl)₂, —N(alkyl)(alkenyl), and —S-alkenyl. The term “heteroalkynyl” as used herein means linear or branched chain alkynyl wherein one or more carbon atoms (usually 1, 2 or 3) are replaced by an oxygen, nitrogen or sulfur atom, with the proviso that said chain may not contain two adjacent O atoms or two adjacent S atoms. The term heteroalkynyl thus comprises -cyano, —O-alkynyl, —NH-alkynyl, —N(alkynyl)₂, —N(alkyl)(alkynyl), —N(alkenyl)(alkynyl), and —S-alkynyl. Unless specified otherwise, a “heteroalkyl” may include between 1-10 carbon atoms/heteroatoms, or in some cases preferably 1-6 carbon atoms/heteroatoms.

The term “heteroalkylene” as used herein encompasses saturated heteroalkylene as well as unsaturated heteroalkylene such as heteroalkenylene, heteroalkynylene, heteroalkenynylene and the like. The term “heteroalkylene” as used herein means linear or branched chain alkylene wherein one or more carbon atoms (usually 1, 2 or 3) are replaced by a heteroatom, i.e., an oxygen, nitrogen or sulfur atom, with the proviso that said chain may not contain two adjacent O atoms or two adjacent S atoms. The term “heteroalkenylene” as used herein means linear or branched chain alkenylene wherein one or more carbon atoms (usually 1, 2 or 3) are replaced by an oxygen, nitrogen or sulfur atom, with the proviso that said chain may not contain two adjacent O atoms or two adjacent S atoms. The term “heteroalkynylene” as used herein means linear or branched chain alkynylene wherein one or more carbon atoms (usually 1, 2 or 3) are replaced by an oxygen, nitrogen or sulfur atom, with the proviso that said chain may not contain two adjacent O atoms or two adjacent S atoms. Unless specified otherwise, a “heteroalkylene” may include between 1-10 carbon atoms/heteroatoms, or in some cases preferably 1-6 carbon atoms/heteroatoms.

The term “carbocycle” as used herein may encompass cycloalkyl, cycloalkenyl, and/or cycloalkynyl. The term “cycloalkyl” as used herein encompasses saturated cycloalkyl as well as unsaturated cycloalkyl such as cycloalkenyl, cycloalkynyl and the like. The term “cycloalkyl” as used herein and unless otherwise stated means a saturated cyclic hydrocarbon radical, such as for instance cyclopropyl, cyclobutyl, cyclopentyl, cyclohexyl, cycloheptyl, cyclooctyl, norbornyl, fenchyl, decalinyl, adamantyl and the like. The term “cycloalkenyl” as used herein means a non-aromatic cyclic hydrocarbon radical with at least one site (usually 1 to 3, preferably 1) of unsaturation, namely a carbon-carbon, sp2 double bond. Examples include, but are not limited to cyclopentenyl and cyclohexenyl. The double bond may be in the cis or trans configuration. The term “cycloalkynyl” as used herein means a non-aromatic cyclic hydrocarbon radical with at least one site (usually 1 to 3, preferably 1) of unsaturation, namely a carbon-carbon, sp triple. An example is cyclohept-1-yne. Unless specified otherwise, a “carbocycle” may include between 3-10 carbon atoms, or in some cases preferably 4-6 carbon atoms.

The term “heterocycle” as used herein may encompass heterocycloalkyls, heterocycloalkenyls, and/or heterocycloalkynyls. The term “heterocycloalkyl” as used herein encompasses saturated heterocycloalkyl as well as unsaturated non-aromatic heterocycloalkyl including at least one heteroatom, i.e., an N, O, or S as ring member. The term “heterocycloalkyl” as used herein and unless otherwise stated means “cycloalkyl” wherein one or more carbon atoms (usually 1, 2 or 3) are replaced by an oxygen, nitrogen or sulfur atom, with the proviso that said chain may not contain two adjacent O atoms or two adjacent S atoms. The term “heterocycloalkenyl” as used herein and unless otherwise stated means “cycloalkenyl” wherein one or more carbon atoms (usually 1, 2 or 3) are replaced by an oxygen, nitrogen or sulfur atom, with the proviso that said chain may not contain two adjacent O atoms or two adjacent S atoms. The term “heterocycloalkynyl” as used herein and unless otherwise stated means “cycloalkynyl” wherein one or more carbon atoms (usually 1, 2 or 3) are replaced by an oxygen, nitrogen or sulfur atom, with the proviso that said chain may not contain two adjacent O atoms or two adjacent S atoms. Examples of saturated and unsaturated heterocycloalkyl include but are not limited to azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, octahydrocyclopenta[c]pyrrole, and hexahydrocyclopenta[c]pyrrole, octahydropyrrolo[1,2-a]pyrazin. Further heterocycloalkyls in the meaning of the invention are described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82: 5566. When the heterocycloalkyl contains no nitrogen as ring member, it is typically bonded through carbon. When the heterocycloalkyl contains nitrogen as ring member, it may be bonded through nitrogen or carbon. Unless specified otherwise, a “heterocycle” may include between 3-10 carbon atoms/heteroatoms, or in some cases preferably 4-6 carbon atoms/heteroatoms.

A “spirocycle” as used herein include two molecular rings sharing only one common atom. One example of a spirocycle is spiro[3.3]heptane. Other examples include any combination of 3-, 4-, 5-, 6-, or 7-membered rings sharing one carbon atom. The term “heterospirocycle” as used herein include two molecular rings sharing only one common carbon atom and further including at least one heteroatom, i.e., an N, O, or S as ring member in either or both of the two molecular rings. Unless specified otherwise, a “spirocycle” may include between 7-14 carbon atoms, or in some cases preferably 8-11 carbon atoms. Unless specified otherwise, a “heterospirocycle” may include between 7-14 carbon atoms/heteroatoms, or in some cases preferably 8-11 carbon atoms/heteroatoms.

The term “fused bicyclic” encompasses any combination of two carbocycle, heterocycle, aromatic (aryl) or heteroaromatic (heteroaryl) groups sharing two adjacent carbon and/or heteroatoms. Unless specified otherwise, a “fused bicyclic” may include between 7-14 carbon atoms/heteroatoms, or in some cases preferably 8-11 carbon atoms/heteroatoms.

The term “bridged bicyclic” generally encompasses any combination of two carbocycle or heterocycle groups sharing two non-adjacent carbon and/or heteroatoms. In some instances, a “bridged bicyclic” may encompass bridged aromatic systems such as bridged annulenes. Unless specified otherwise, a “bridged bicyclic” may include between 7-14 carbon atoms/heteroatoms, or in some cases preferably 8-11 carbon atoms/heteroatoms.

The term “aromatic,” or alternatively “aryl,” as used herein means an aromatic hydrocarbon. Typical aromatic or aryl groups include, but are not limited to 1 ring, or 2 or 3 rings fused together, radicals derived from benzene, naphthalene, anthracene, biphenyl, and the like. Unless specified otherwise, an “aromatic” group may include between 5-8 carbon atoms, or in some cases preferably 5-6 carbon atoms.
The term “heteroaromatic,” or alternatively “heteroaryl,” as used herein means an aromatic ring system including at least one heteroatom, i.e., N, O, or S as ring member of the aromatic ring system. Examples of heteroaryl include but are not limited to benzimidazole, benzisoxazole, benzoazole, benzodioxole, benzofuran, benzothiadiazole, benzothiazole, benzothiophene, carbazole, cinnoline, dibenzofuran, furane, furazane, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine. Unless specified otherwise, a “heteroaromatic” group may include between 5-8 carbon atoms/heteroatoms, or in some cases preferably 5-6 carbon atoms/heteroatoms.
By further way of example, carbon bonded heterocyclic rings are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.
Carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl. By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of an isoindole, or isoindoline, position 4 of a morpholine, and position 9 of a carbazole, or β-carboline. Nitrogen bonded heterocycles include 1-aziridyl, 1-azetedyl, 1-pyrrolyl, 1-imidazolyl, 1-pyrazolyl, and 1-piperidinyl. Further heteroaryls in the meaning of the invention are described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82: 5566.
As used herein with respect to a substituting group, and unless otherwise stated, the terms “monosubstituted”, “disubstituted”, “trisubstituted”, “polysubstituted” and the like means chemical structures defined herein, wherein the respective moiety is substituted with one or more substituents, meaning that one or more hydrogen atoms of said moiety are each independently replaced with a substituent. For example, -C₁₋₆-alkyl that may be polysubstituted with-F includes —CH₂F, —CHF₂, —CF₃, —CH₂CF₃, CF₂CF₃, and the like. Likewise, -C₁₋₆-alkyl that may be polysubstituted with substituents independently of one another selected from —F and —Cl includes —CH₂F, —CHF₂, —CF₃, —CH₂CF₃, CF₂CF₃, —CH₂Cl, —CHCl₂, —CCl₃, —CH₂CCl₃, CCl₂CCl₃, —CHClF, —CClF₂, —CCl₂CF₃, —CF₂CCl₃, —CClFCCl₂F, and the like. Any substituent designation that is found in more than one site in a compound of this invention shall be independently selected.

### Dosages and Treatment Regimens

In some embodiments, the pharmaceutical compositions comprise from about 1 μg to about 1000 mg per unit dosage of the compound based on the active moiety of the compound. The compositions can comprise about 1 μg, 100 μg, 1 mg, 10 mg, 100 mg, or about 200 mg, or about 300 mg, or about 400 mg, or about 500 mg or about 600 mg, or about 700 mg, or about 800 mg per unit dosage of the compound based on the active moiety of the compound.

In some embodiments, a dosing range for the compound capable of inhibiting PRMT5 based on the active moiety of the compound is from about 0.001 μg/kg to about 1000 mg/kg of body weight/per day of the subject.

The dosage can be varied to achieve an amount of the active ingredient that is effective for obtaining the desired therapeutic effect.

The target indication of the invention composition is related to methods of treating cancers and infectious disease. The compositions of the present invention can be administered according to a variety of regimens.

In an embodiment, provided for are methods of treating a disease state by administration of one or more compounds of the present disclosure to a mammalian subject in need thereof. In an embodiment, provided for are methods of treating a cancer by administration of one or more compounds of the present disclosure to a mammalian subject in need thereof. In an embodiment, provided for are methods of treating a cancer by administration of one or more of compounds 1-105 of the present disclosure to a mammalian subject in need thereof. In an embodiment, the cancer is any cancer, cancerous cell, or malignancy which responds to administration of one or more of compounds 1-105 as would be appreciated by a person skilled in the art. In an embodiment, the cancer, cancerous cell, or malignancy is lymphoma. In an embodiment, provided for are methods for treating lymphoma by administration of one or more compounds of the present disclosure to a mammalian subject in need thereof. In an embodiment, provided for are methods of treating lymphoma by administration of one or more of compounds 1-105 of the present disclosure to a mammalian subject in need thereof.

### Combination Therapies

In other embodiments, the patient or subject can be administered at least one additional active agent for treating, preventing or reducing the severity of the cancer.

### Formulations

In the present invention other optional ingredients may also be incorporated into the pharmaceutical compositions. Such ingredients can include, for example, pharmaceutically acceptable excipients and preservatives. The excipients that can be used in accordance with the present invention include, for example, bio-adhesives and/or swelling/thickening agents.

In the present invention, any other suitable absorption enhancers as known in the art may also be used.

Preservatives can also be added to the present compositions. Suitable preservatives that can be used with the present compositions include, for example, benzyl alcohol, parabens, thimerosal, chlorobutanol and benzalkonium, with benzalkonium chloride being preferred. Typically, the preservative will be present in the present compositions in a concentration of up to about 2% by weight. The exact concentration of the preservative, however, will vary depending upon the intended use and can be easily ascertained by one skilled in the art.

The absorption enhancing agent includes (i) a surfactant; (ii) a bile salt (including sodium taurocholate); (iii) a phospholipid additive, mixed micelle, or liposome; (iv) an alcohol (including a polyol as discussed above, for example, propylene glycol or polyethylene glycol such as PEG 3000, etc.); (v) an enamine; (vi) a nitric oxide donor compound; (vii) a long-chain amphipathic molecule; (viii) a small hydrophobic uptake enhancer; (ix) sodium or a salicylic acid derivative; (x) a glycerol ester of acetoacetic acid; (xi) a cyclodextrin or cyclodextrin derivative; (xii) a medium-chain or short-chain (e.g. Cl to C 12) fatty acid; and (xiii) a chelating agent; (xiv) an amino acid or salt thereof; and (xv) an N-acetylamino acid or salt thereof.

Solubility enhancers may increase the concentration of the drug or pharmaceutically acceptable salt thereof in the formulation. Useful solubility enhancers include, e.g., alcohols and polyalcohols.

An isotonizing agent may improve the tolerance of the formulations. A common isotonizing agent is NaCl. For example, when the formulation is an isotonic intranasal dosage formulation, it includes about 0.9% NaCl (v/v) in the aqueous portion of the liquid carrier.

The thickeners may improve the overall viscosity of the composition. Suitable thickeners include methylcellulose, carboxymethylcellulose, polyvinylpyrrolidone, sodium alginate, hydroxypropylmethylcellulose, and chitosan.

A humectant or anti-irritant improves the tolerability of the composition in repeated applications. Suitable compounds include, e.g. glycerol, tocopherol, mineral oils, and chitosan.

Various additional ingredients can be used in the compositions of the present invention. The compositions can comprise one or more further ingredients selected from a preservative, an antioxidant, an emulsifier, a surfactant or wetting agent, an emollient, a film-forming agent, or a viscosity modifying agent. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In another aspect, suitable propellants can be used for dispensing the product when in the form of a liquid or powder for delivery from a spray device or nebulizer.

In one aspect, a preservative can be included. In another aspect, an antioxidant can be included. In another aspect, an emulsifier can be included. In another aspect, an emollient can be included. In another aspect, a viscosity modifying agent can be included. In another aspect, a surfactant or wetting agent can be included. In another aspect, a film forming agent can be included. In another aspect, the pharmaceutical composition is in the form selected from the group consisting of a gel, ointment, lotion, emulsion, cream, liquid, spray, suspension, jelly, foam, mousse, paste, tape, dispersion, aerosol. These components can be employed and used at levels appropriate for the formulation based on the knowledge of one with ordinary skill in the pharmaceutical and formulation arts.

In another aspect, the at least one preservative can be selected from the group consisting of parabens (including butylparabens, ethylparabens, methylparabens, and propylparabens), acetone sodium bisulfite, alcohol, benzalkonium chloride, benzethonium chloride, benzoic acid, benzyl alcohol, boric acid, bronopol, butylated hydroxyanisole, butylene glycol, calcium acetate, calcium chloride, calcium lactate, cetrimide, cetylpyridinium chloride, chlorhexidine, chlorobutanol, chlorocresol, chloroxylenol, cresol, edetic acid, glycerin, hexetidine, imidurea, isopropyl alcohol, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium nitrate, potassium sorbate, propionic acid, propyl gallate, propylene glycol, propylparaben sodium, sodium acetate, sodium benzoate, sodium borate, sodium lactate, sodium metabisulfite, sodium propionate, sodium sulfite, sorbic acid, sulfur dioxide, thimerosal, zinc oxide, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one antioxidant can be selected from the group consisting of acetone sodium bisulfite, alpha tocopherol, ascorbic acid, ascorbyl palmitate, butylated hydroxyanisole, butylated hydroxytoluene, citric acid monohydrate, dodecyl gallate, erythorbic acid, fumaric acid, malic acid, mannitol, sorbitol, monothioglycerol, octyl gallate, potassium metabisulfite, propionic acid, propyl gallate, sodium ascorbate, sodium formaldehyde sulfoxylate, sodium metabisulfite, sodium sulfite, sodium thiosulfate, sulfur dioxide, thymol, vitamin E polyethylene glycol succinate, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emulsifier can be selected from the group consisting of acacia, agar, ammonium alginate, calcium alginate, carbomer, carboxymethylcellulose sodium, cetostearyl alcohol, cetyl alcohol, cholesterol, diethanolamine, glyceryl monooleate, glyceryl monostearate, hectorite, hydroxypropyl cellulose, hydroxypropyl starch, hypromellose, lanolin, lanolin alcohols, lauric acid, lecithin, linoleic acid, magnesium oxide, medium-chain triglycerides, methylcellulose, mineral oil, monoethanolamine, myristic acid, octyldodecanol, oleic acid, oleyl alcohol, palm oil, palmitic acid, pectin, phospholipids, poloxamer, polycarbophil, polyoxyethylene alkyl ethers, polyoxyethylene castor oil derivatives, polyoxyethylene sorbitan fatty acid esters, polyoxyethylene stearates, polyoxyl 15 hydroxystearate, polyoxyglycerides, potassium alginate, propylene glycol alginate, propylene glycol dilaurate, propylene glycol monolaurate, saponite, sodium borate, sodium citrate dehydrate, sodium lactate, sodium lauryl sulfate, sodium stearate, sorbitan esters, starch, stearic acid, sucrose stearate, tragacanth, triethanolamine, tromethamine, vitamin E polyethylene glycol succinate, wax, and xanthan gum, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emollient can be selected from the group consisting of almond oil, aluminum monostearate, butyl stearate, canola oil, castor oil, cetostearyl alcohol, cetyl alcohol, cetyl palmitate, cholesterol, coconut oil, cyclomethicone, decyl oleate, diethyl sebacate, dimethicone, ethylene glycol stearates, glycerin, glyceryl monooleate, glyceryl monostearate, isopropyl isostearate, isopropyl myristate, isopropyl palmitate, lanolin, lanolin alcohols, lecithin, mineral oil, myristyl alcohol, octyldodecanol, oleyl alcohol, palm kernel oil, palm oil, petrolatum, polyoxyethylene sorbitan fatty acid esters, propylene glycol dilaurate, propylene glycol monolaurate, safflower oil, squalene, sunflower oil, tricaprylin, triolein, wax, xylitol, zinc acetate, or a combination thereof. These components can be employed and used at levels appropriate for the formulation based on the knowledge of one with ordinary skill in the pharmaceutical and formulation arts. The amounts could range from under 1 percent by weight to up to 60 percent by weight.

In another aspect, the at least one viscosity modifying agent can be selected from the group consisting of acacia, agar, alginic acid, aluminum monostearate, ammonium alginate, attapulgite, bentonite, calcium alginate, calcium lactate, carbomer, carboxymethylcellulose calcium, carboxymethylcellulose sodium, carrageenan, cellulose, ceratonia, ceresin, cetostearyl alcohol, cetyl palmitate, chitosan, colloidal silicon dioxide, corn syrup solids, cyclomethicone, ethylcellulose, gelatin, glyceryl behenate, guar gum, hectorite, hydrophobic colloidal silica, hydroxyethyl cellulose, hydroxyethylmethyl cellulose, hydroxypropyl cellulose, hydroxypropyl starch, hypromellose, magnesium aluminum silicate, maltodextrin, methylcellulose, myristyl alcohol, octyldodecanol, palm oil, pectin, polycarbophil, polydextrose, polyethylene oxide, polyoxyethylene alkyl ethers, polyvinyl alcohol, potassium alginate, propylene glycol alginate, pullulan, saponite, sodium alginate, starch, sucrose, sugar, sulfobutylether β-cyclodextrin, tragacanth, trehalose, and xanthan gum, or a combination thereof. These components can be employed and used at levels appropriate for the formulation based on the knowledge of one with ordinary skill in the pharmaceutical and formulation arts. The amounts could range from under 1 percent by weight to up to 60 percent.

In another aspect, the at least one film forming agent can be selected from the group consisting of ammonium alginate, chitosan, colophony, copovidone, ethylene glycol and vinyl alcohol grafted copolymer, gelatin, hydroxypropyl cellulose, hypromellose, hypromellose acetate succinate, polymethacrylates, poly (methyl vinyl ether/maleic anhydride), polyvinyl acetate dispersion, polyvinyl acetate phthalate, polyvinyl alcohol, povidone, pullulan, pyroxylin, and shellac, or a combination thereof. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In another aspect, the at least one surfactant or wetting agent can be selected from the group consisting of docusate sodium, phospholipids, sodium lauryl sulfate, benzalkonium chloride, cetrimide, cetylpyridinium chloride, alpha tocopherol, glyceryl monooleate, myristyl alcohol, poloxamer, polyoxyethylene alkyl ethers, polyoxyethylene castor oil derivatives, polyoxyethylene sorbitan fatty acid esters, polyoxyethylene stearates, polyoxyl 15 hydroxystearate, polyoxyglycerides, propylene glycol dilaurate, propylene glycol monolaurate, sorbitan esters, sucrose stearate, tricaprylin, and vitamin

E polyethylene glycol succinate, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, a buffering agent can be included. In another aspect, an emollient can be included. In another aspect, an emulsifying agent can be included. In another aspect, an emulsion stabilizing agent can be included. In another aspect, a gelling agent can be included. In another aspect, a humectant can be included. In another aspect, an ointment base or oleaginous vehicle can be included. In another aspect, a suspending agent can be included. In another aspect an acidulant can be included. In another aspect, an alkalizing agent can be included. In another aspect, a bioadhesive material can be included. In another aspect, a colorant can be included. In another aspect, a microencapsulating agent can be included. In another aspect, a stiffening agent can be included. These components can be employed and used at levels appropriate for the formulation based on the knowledge of one with ordinary skill in the pharmaceutical and formulation arts. The amounts could range from under 1 percent by weight to up to 90 percent or even over 99 by weight.

One of ordinary skill in the pharmaceutical and formulation arts can determine the appropriate levels of the essential and optional components of the compositions of the present invention.

The compositions of the present invention can be in a variety of forms including oral and intravenous forms, topical forms and also parenteral forms and compositions for injection, and compounds for infusion. Additionally, the following routes of administration are contemplated: nasal, inhalation (i.e. respiratory), intratracheal, intrapulmonary, and intrabronchial.

The compositions can be in the form of liquids, suspensions or dry powders. These compositions can be delivered into the lungs via a nebulizer or atomizer. The present invention also contemplates devices for spraying the compositions and kits comprising such a delivery device and instructions for use.

In an embodiment, one or more compounds of the present disclosure may be administered by any appropriate route of administration. In an embodiment, compounds 1-105 may be administered by any appropriate route of administration to a mammalian subject in need thereof. In an embodiment, the route of administration is oral. In an embodiment, the route of administration is intravenous. In an embodiment, the route of administration is parenteral. In an embodiment, the route of administration is intratumor (i.e. directly injected into a tumor, tumorous cells, or a malignancy) and intra-arterial.

Methods of preparing the compositions are also intended as part of the present invention and would be apparent to one of ordinary skill in the pharmaceutical and formulation arts using standard formulation and mixing techniques.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### General Synthetic Methods

The compounds of the invention may be prepared by the methods described below. Optimum reaction conditions and reaction times may vary depending on the particular reactants used. Unless otherwise specified, solvents, temperatures, pressures and other reaction conditions may be readily selected by one of ordinary skill in the art. Specific procedures are provided in the Synthetic Examples section. Typically, reaction progress may be monitored by thin layer chromatography (TLC) or HPLC-MS if desired. Intermediates and products may be purified by chromatography on silica gel, recrystallization, HPLC and/or reverse phase HPLC.

Starting materials and reagents are either commercially available or may be prepared by one skilled in the art using methods described in the chemical literature and in the Synthetic Examples section below.

### Example 1: Compound 2

3-[2-hydroxy-3-(1,2,3,4-tetrahydroisoquinolin-2-yl)propyl]-1-(2-methylpropyl)-1,2,3,4-tetrahydroquinazoline-2,4-dione

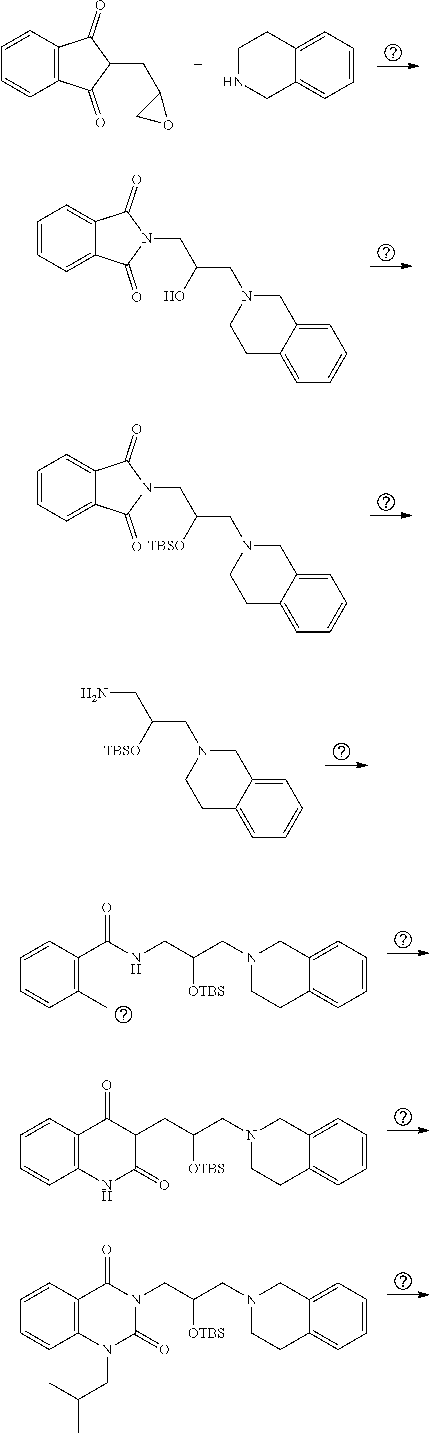

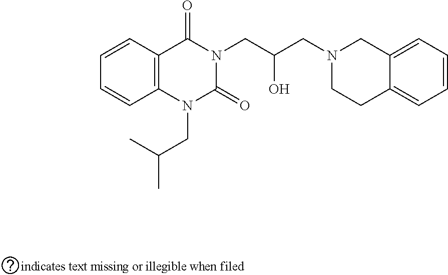 [SMILES: CC(C)Cn1c(=O)n(CC(O)CN2CCc3ccccc3C2)c(=O)c2ccccc21]

### Step 1

2-[2-Hydroxy-3-(1,2,3,4-tetrahydroisoquinolin-2-yl)propyl]-2,3-dihydro-1H-isoindole-1,3-dione
To a stirred suspension of 2-(oxirian-2-ylmethyl)isoindoline-1,3-dione (5 g, 24 mmol) in EtOH (10 mL) was added 1,2,3,4-tetrahydroisoquinoline (4.8 g, 36 mmol). The resulting mixture was warmed to 80° C. and stirred overnight. After this time, the homogenous reaction was cooled to RT and concentrated in vacuo. The crude product (7.6 g) was used without further purification.

### Step 2

2-{2-[(tert-Butyldimethylsilyl)oxy]-3-(1,2,3,4-tetrahydroisoquinolin-2-yl)propyl}-2,3-dihydro-1H-isoindole-1,3-dione
To a stirred solution of the above crude intermediate (7.6 g, 23 mmol) in DMF (10 mL) was added tert-butyldimethylsilyl chloride (4.1 g, 28 mmol), imidazole (1.9 g, 28 mmol) and DMAP (280 mg, 2.3 mmol). The reaction was stirred overnight at room temperature and then poured into water. The product was extracted into ethyl acetate (4×10 mL), concentrated to low volume and washed with water. The organic was washed with brine and dried (MgSO₄). Filtration and concentration gave the crude product which was purified via silica gel flash chromatography using 0-30% ethyl acetate/hexane as eluent to obtain 2-{2-[(tert-Butyldimethylsilyl)oxy]-3-(1,2,3,4-tetrahydroisoquinolin-2-yl)propyl}-2,3-dihydro-1H-isoindole-1,3-dione (9.3 g, 90%)

### Step 3

2-{3-Amino-2-[(tert-butyldimethylsilyl)oxy]propyl}-1,2,3,4-tetrahydroisoquinoline
To a stirred solution of 2-{2-[(tert-Butyldimethylsilyl)oxy]-3-(1,2,3,4-tetrahydroisoquinolin-2-yl)propyl}-2,3-dihydro-1H-isoindole-1,3-dione (16.3 g, 36.2 mmol) in MeOH (70 ml) was added hydrazine monohydrate (5.4 g, 108.6 mmol). The resulting solution was warmed to 50° C. for 40 minutes during which time a precipitate formed. Water (8 ml) was added, and the reaction was allowed to cool to room temperature overnight. The solids were removed via filtration, transferring the mixture with CH₂Cl₂. The combined filtrates were concentrated to precipitate more by-product. The mixture was diluted with CH₂Cl₂ and the solids were again removed via filtration. The filtrate was concentrated and transferred to a separatory funnel with CH₂Cl₂. The organic was washed with brine and dried (MgSO₄). The filtrate was concentrated to give the product which was used without further purification (8.3 g).

### Step 4

2-Amino-N-{2-[(tert-Butyldimethylsilyl)oxy]-3-(1,2,3,4-tetrahydroisoquinolin-2-yl)propyl}benzamide
To a stirred solution of 2-{3-Amino-2-[(tert-butyldimethylsilyl)oxy]propyl}-1,2,3,4-tetrahydroisoquinoline (0.25 g, 0.8 mmol) in CH₂Cl₂ (1 ml) was added 1H-benzo[d][1,3]oxazine-2,4-dione (0.13 g, 0.8 mmol). Upon complete addition, gas evolution was allowed to subside after which time the reaction was placed in a sand bath which was pre-heated to 150° C. After five minutes the reaction was cooled to room temperature. The crude residue was purified via silica gel flash column chromatography using 0-30% ethyl acetate/hexane as the eluent to give 2-Amino-N-{2-[(tert-Butyldimethylsilyl)oxy]-3-(1,2,3,4-tetrahydroisoquinolin-2-yl)propyl}benzamide (0.30 g, 85%)

### Step 5

3-{2-[(tert-Butyldimethylsilyl)oxy]-3-(1,2,3,4-tetrahydroisoquinolin-2-yl)propyl}-1,2,3,4-tetrahydroquinazoline-2,4-dione
To a stirred solution of 2-Amino-N-{2-[(tert-Butyldimethylsilyl)oxy]-3-(1,2,3,4-tetrahydroisoquinolin-2-yl)propyl}benzamide (0.3 g, 0.7 mmol) in THF (3 ml) was added DBU (0.2 g, 1.3 mmol) and 1,1′-Carbonyldiimidazole (CDI) (0.2 g, 1.3 mmol). The resulting mixture was warmed to 50° C. for 1 h after which time it was cooled to room temperature. The reaction was poured into water and the product was extracted into CH₂Cl₂ (3×10 mL). The combined organics were dried (MgSO₄), filtered and concentrated. The crude was purified via silica gel flash column chromatography using 0-30% ethyl acetate/hexane as the eluent to give 3-{2-[(tert-Butyldimethylsilyl)oxy]-3-(1,2,3,4-tetrahydroisoquinolin-2-yl)propyl}-1,2,3,4-tetrahydroquinazoline-2,4-dione as a white solid (0.27 g, 45%).

### Step 6

3-{2-[(tert-Butyldimethylsilyl)oxy]-3-(1,2,3,4-tetrahydroisoquinolin-2-yl)propyl}-1-(2-methylpropyl)-1,2,3,4-tetrahydroquinazoline-2,4-dione
To a stirred solution of 3-{2-[(tert-Butyldimethylsilyl)oxy]-3-(1,2,3,4-tetrahydroisoquinolin-2-yl)propyl}-1,2,3,4-tetrahydroquinazoline-2,4-dione (0.1 g, 0.2 mmol) in DMF (3 ml) was added K₂CO₃ (90 mg, 0.6 mmol) and 1-bromo-2-methylpropane (32 mg, 0.2 mmol). The reaction was warmed to 70° C. for 5 h then cooled to room temperature and stirred overnight. The reaction was poured into water and the product was extracted into ethyl acetate (3×). The crude was purified via silica gel flash column chromatography using 0-20% ethyl acetate/hexane as the eluent to give 3-{2-[(tert-Butyldimethylsilyl)oxy]-3-(1,2,3,4-tetrahydroisoquinolin-2-yl)propyl}-1-(2-methylpropyl)-1,2,3,4-tetrahydroquinazoline-2,4-dione as a white foam (55 mg, 52%).

### Step 7

3-[2-hydroxy-3-(1,2,3,4-tetrahydroisoquinolin-2-yl)propyl]-1-(2-methylpropyl)-1,2,3,4-tetrahydroquinazoline-2,4-dione
To a stirred solution of 3-{2-[(tert-Butyldimethylsilyl)oxy]-3-(1,2,3,4-tetrahydroisoquinolin-2-yl)propyl}-1-(2-methylpropyl)-1,2,3,4-tetrahydroquinazoline-2,4-dione (50 mg, 0.09 mmol) in THF (1 ml) was added tetrabutylammonium fluoride (1M in THF, 0.2 ml, 0.2 mmol). The reaction was stirred at room temperature overnight and concentrated. The remaining residue was dissolved into CH₂Cl₂ and washed with water (10 mL). The aqueous phase was extracted with CH₂Cl₂ (10 mL) and the combined organics were dried (MgSO₄), filtered and concentrated. The crude was purified via silica gel flash column chromatography using 0-50% ethyl acetate/CH₂Cl₂ as the eluent to give 3-[2-hydroxy-3-(1,2,3,4-tetrahydroisoquinolin-2-yl)propyl]-1-(2-methylpropyl)-1,2,3,4-tetrahydroquinazoline-2,4-dione as a white solid (30 mg, 83%). LCMS (ESMS): m/z: 408.3 (M⁺+1)
The following compounds were prepared in a similar manner:

### Compound 1

1-[(4-Chlorophenyl)methyl]-3-[2-hydroxy-3-(1,2,3,4-tetrahydroisoquinolin-2-yl)propyl]-1,2,3,4-tetrahydroquinazoline-2,4-dione. LCMS (ESMS): m/z: 476.2 (M⁺+1)

### Compound 3

3-[2-Hydroxy-3-(1,2,3,4-tetrahydroisoquinolin-2-yl)propyl]-1-[2-(4-methoxyphenyl)ethyl]-1,2,3,4-tetrahydroquinazoline-2,4-dione, LCMS (ESMS): m/z: 486.3 (M⁺+1)

The remaining compounds 4-105 can be synthesized and characterized in accordance with the disclosed procedures, adapted as necessary as understood by a person of skill in the art.

### Example 2: Compound 76

Synthesis of 6-(4-fluorophenyl)-3-[(2R)-2-hydroxy-3-(1,2,3,4-tetrahydroisoquinolin-2-yl)propyl]-1-(2-methylpropyl)-1,2,3,4-tetrahydroquinazoline-2,4-dione (Compound 76)

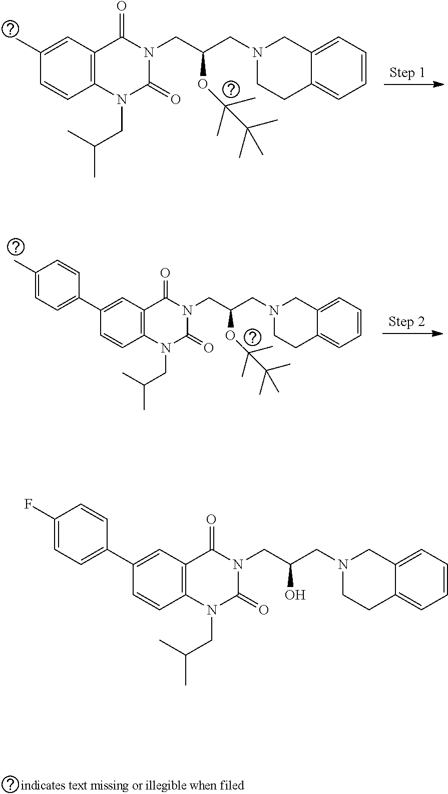 [SMILES: Cc1ccc2c(c1)c(=O)n(C[C@@H](CN1CCc3ccccc3C1)OC(C)(C)C(C)(C)C)c(=O)n2CC(C)C ; Cc1ccc(-c2ccc3c(c2)c(=O)n(C[C@@H](CN2CCc4ccccc4C2)OC(C)(C)C(C)(C)C)c(=O)n3CC(C)C)cc1 ; CC(C)Cn1c(=O)n(C[C@H](O)CN2CCc3ccccc3C2)c(=O)c2cc(-c3ccc(F)cc3)ccc21]

### Step 1

3-[(2R)-2-[(tert-butyldimethylsilyl)oxy]-3-(1,2,3,4-tetrahydroisoquinolin-2-yl)propyl]-6-(4-fluorophenyl)-1-(2-methylpropyl)-1,2,3,4-tetrahydroquinazoline-2,4-dione
A sealed tube was charged with 6-bromo-3-[(2R)-2-[(tert-butyldimethylsilyl)oxy]-3-(1,2,3,4-tetrahydroisoquinolin-2-yl)propyl]-1-(2-methylpropyl)-1,2,3,4-tetrahydroquinazoline-2,4-dione (0.2 g, 0.4 mmol), 4-fluorophenylboronic acid (66 mg, 0.44 mmol) and THF (4ml). Added 2M Na₂CO₃ (0.74 ml) and Pd (PPh₃)₄ (43 mg, 0.04 mmol). The resulting mixture was capped and stirred at 80° C. overnight. Upon cooling to room temperature, the reaction was poured into water and extracted with ethyl acetate (3×). The combined organics were dried (MgSO₄), filtered and concentrated. The crude product was purified via flash column chromatography using 0-30% ethyl acetate/hexane as eluent to afford 3-[(2R)-2-[(tert-butyldimethylsilyl)oxy]-3-(1,2,3,4-tetrahydroisoquinolin-2-yl)propyl]-6-(4-fluorophenyl)-1-(2 -methylpropyl)-1,2,3,4-tetrahydroquinazoline-2,4-dione (60 mg, 26%).

### Step 2

6-(4-fluorophenyl)-3-[(2R)-2-hydroxy-3-(1,2,3,4-tetrahydroisoquinolin-2-yl)propyl]-1-(2-methylpropyl)-1,2,3,4-tetrahydroquinazoline-2,4-dione
Prepared in a manner described in Step 7 of Example 1
The following compounds were prepared in a similar manner:
1-(cyclopropylmethyl)-3-[(2R)-2-hydroxy-3-(1,2,3,4-tetrahydroisoquinolin-2-yl)propyl]-7-(pyridin-3-yl)-1,2,3,4-tetrahydroquinazoline-2,4-dione (Compound 86)
1-(cyclopropylmethyl)-3-[(2R)-2-hydroxy-3-(1,2,3,4-tetrahydroisoquinolin-2-yl)propyl]-7-(6-methoxypyridin-3-yl)-1,2,3,4-tetrahydroquinazoline-2,4-dione (Compound 91)
1-(2,2-difluoroethyl)-6-(4-fluorophenyl)-3-[(2R)-2-hydroxy-3-(1,2,3,4-tetrahydroisoquinolin-2-yl)propyl]-1,2,3,4-tetrahydroquinazoline-2,4-dione (Compound 94)

The remaining compounds of Table 1 can be synthesized and characterized in accordance with the disclosed procedures, adapted as necessary as understood by a person of skill in the art.

TABLE 3
Characterization Data

| | |
|---|---|
| Compound 61 | Compound 62 |
| LCMS (ESMS): m/z: | LCMS (ESMS): m/z: |
| 352.3 (M⁺ + 1) | 352.4 (M⁺ + 1) |
| Compound 63 | Compound 64 |
| LCMS (ESMS): m/z: | LCMS (ESMS): m/z: |
| 366.4 (M⁺ + 1) | 352.4 (M⁺ + 1) |
| Compound 65 | Compound 66 |
| LCMS (ESMS): m/z: | LCMS (ESMS): m/z: |
| 409.5 (M⁺ + 1) | 370.3 (M⁺ + 1) |
| Compound 67 | Compound 68 |
| LCMS (ESMS): m/z: | LCMS (ESMS): m/z: |
| 386.8 (M⁺ + 1) | 386.8 (M⁺ + 1) |
| Compound 69 | Compound 70 |
| LCMS (ESMS): m/z: | LCMS (ESMS): m/z: |
| 442.9 (M⁺ + 1) | 487.4 (M⁺ + 1) |
| Compound 71 | Compound 72 |
| LCMS (ESMS): m/z: | LCMS (ESMS): m/z: |
| 487.4 (M⁺ + 1) | 426.4 (M⁺ + 1) |
| Compound 73 | Compound 74 |
| LCMS (ESMS): m/z: | LCMS (ESMS): m/z: |
| 442.9 (M⁺ + 1) | 406.4 (M⁺ + 1) |
| Compound 75 | Compound 76 |
| LCMS (ESMS): m/z: | LCMS (ESMS): m/z: |
| 431.2 (M⁺ + 1) | 502.9 (M⁺ + 1) |
| Compound 77 | Compound 78 |
| LCMS (ESMS): m/z: | LCMS (ESMS): m/z: |
| 424.4 (M⁺ + 1) | 440.9 (M⁺ + 1) |
| Compound 79 | Compound 80 |
| LCMS (ESMS): m/z: | LCMS (ESMS): m/z: |
| 416.4 (M⁺ + 1) | 485.3 (M⁺ + 1) |
| Compound 81 | Compound 82 |
| LCMS (ESMS): m/z: | LCMS (ESMS): m/z: |
| 382.4 (M⁺ + 1) | 382.4 (M⁺ + 1) |
| Compound 83 | Compound 84 |
| LCMS (ESMS): m/z: | LCMS (ESMS): m/z: |
| 368.3 (M⁺ + 1) | 436.5 (M⁺ + 1) |
| Compound 85 | Compound 86 |
| LCMS (ESMS): m/z: | LCMS (ESMS): m/z: |
| 420.5 (M⁺ + 1) | 483.5 (M⁺ + 1) |
| Compound 87 | Compound 88 |
| LCMS (ESMS): m/z: | LCMS (ESMS): m/z: |
| 436.5 (M⁺ + 1) | 420.5 (M⁺ + 1) |
| Compound 89 | Compound 90 |
| LCMS (ESMS): m/z: | LCMS (ESMS): m/z: |
| 424.4 (M⁺ + 1) | 424.4 (M⁺ + 1) |
| Compound 91 | Compound 92 |
| LCMS (ESMS): m/z: | LCMS (ESMS): m/z: |
| 513.5 (M⁺ + 1) | 420.5 (M⁺ + 1) |
| Compound 93 | Compound 94 |
| LCMS (ESMS): m/z: | LCMS (ESMS): m/z: |
| 462.4 (M⁺ + 1) | 510.5 (M⁺ + 1) |
| Compound 95 | Compound 96 |
| LCMS (ESMS): m/z: | LCMS (ESMS): m/z: |
| 456.4 (M⁺ + 1 | 476.9 (M⁺ + 1) |
| Compound 97 | Compound 98 |
| LCMS (ESMS): m/z: | LCMS (ESMS): m/z: |
| 472.5 (M⁺ + 1) | 408.5 (M⁺ + 1) |
| Compound 99 | Compound 100 |
| LCMS (ESMS): m/z: | LCMS (ESMS): m/z: |
| 486.5 (M⁺ + 1) | 467.5 (M⁺ + 1) |
| Compound 101 | Compound 102 |
| LCMS (ESMS): m/z: | LCMS (ESMS): m/z: |
| 408.5 (M⁺ + 1) | 408.5 (M⁺ + 1) |
| Compound 103 | Compound 104 |
| LCMS (ESMS): m/z: | LCMS (ESMS): m/z: |
| 486.5 (M⁺ + 1) | 486.5 (M⁺ + 1) |
| Compound 105 | |
| LCMS (ESMS): m/z: | |
| 450.5 (M⁺ + 1) | |

### Example 3: PRMT5 Assay

The PRMT5 activity is measured by using “PRMT5 Chemiluminescent Assay Kit” from BPS Bioscience, Catalog Number 52002L as per the instructions of the manufacturer. Briefly, the PRMT5 enzyme is incubated with S-adenosylmethionine in a 96-well plate precoated with histone H4 peptide substrate. Next, a highly specific antibody that recognizes methylated R3 residue of Histone H4 is added followed by a horseradkish peroxidase-labeled (HRP-labeled) secondary antibody. Detection is done by the addition of the HRP substrate to produce chemiluminescence that can be measured quantitatively.

Compounds were serially diluted 3-fold and dosed from 10 uM down to 0 uM. Compounds were tested in duplicates to generate an eight-point dose response for the calculation of their IC50s.

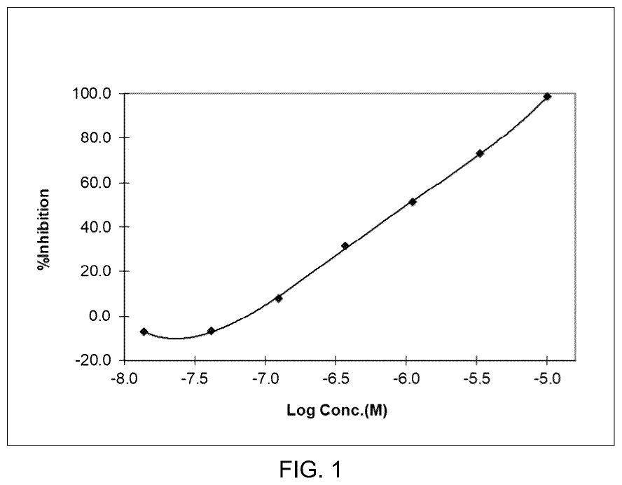

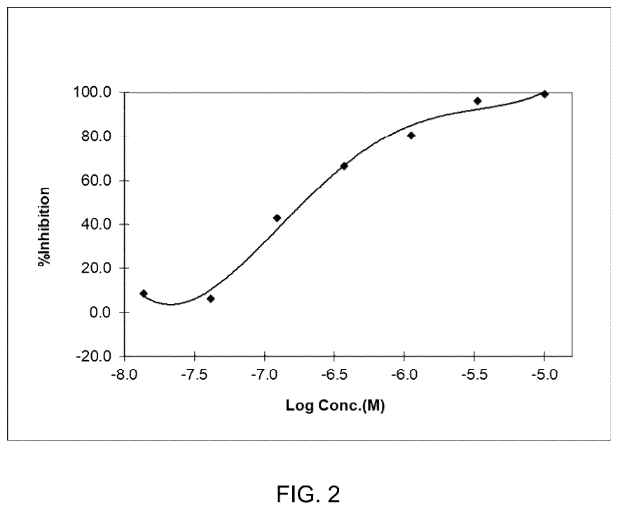

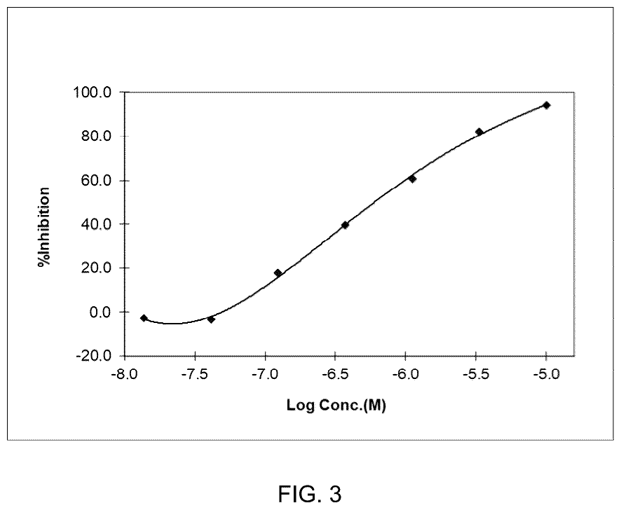

Referring to FIGS. 1, 2, and 3, plots of PRMT5 activity are shown as described above. The compounds 1, 2, and 3 were found to have the following IC50 values as shown in Table 4.

TABLE 4
PRMT5 Assay Results

| | IC50 Values |
|---|---|
| Compound | μMolar |
| 61 | 0.876 |
| 96 | 1.064 |
| 97 | 9.414 |
| 98 | 0.178 |
| 99 | 0.641 |
| 100 | 0.779 |
| 62 | 13.358 |
| 63 | 0.698 |
| 101 | 4.488 |
| 64 | 0.360 |
| 102 | 0.071 |
| 65 | 0.518 |
| 103 | 6.537 |
| 104 | 0.172 |
| 105 | 0.903 |
| 66 | 0.176 |
| 67 | 0.216 |
| 68 | 0.033 |
| 69 | 0.126 |
| 70 | <0.014 |
| 71 | 0.159 |
| 72 | 0.07 |
| 73 | 0.014 |
| 74 | <0.014 |
| 75 | 0.024 |
| 76 | 0.057 |
| 77 | 0.063 |
| 78 | 0.012 |
| 79 | 0.029 |
| 80 | 0.009 |
| 81 | 0.159 |
| 82 | 0.066 |
| 83 | 0.84 |
| 84 | 0.035 |
| 85 | 0.029 |
| 86 | 0.035 |
| 87 | 0.058 |
| 88 | 0.021 |
| 89 | 0.041 |
| 90 | 0.121 |
| 91 | 0.043 |
| 92 | 0.036 |
| 93 | 0.122 |
| 94 | 0.045 |
| 95 | 0.164 |

### Example 4: Z138 Cell Proliferation Assay

### Materials for Assay

Z-138 cells from ATCC Cat No. CRL-3001; Lot No. 70003027

### Human Mantle Cell Lymphoma

Z-138 cells were plated in a 96-well plate in IMDM medium containing 10% Horse Serum and PenStrep at a density of 10,000 cells/well. The plates were incubated at 37° C. in a humidified, 5% CO₂ incubator. The next day, serially diluted compounds were added in duplicates for 72 hours. Compounds were tested at 10, 2.5, 0.625, 0.156, 0.039, 0.010 0.002 and 0 uM final concentrations. After 72 hours of compound addition, 100 ml of Promega's CTG assay reagent was added and incubated at room temperature for 10 minutes. Chemiluminescence was read using a GloMax Discover instrument.

These results demonstrate that exemplary compounds of the present invention, including compounds 1-105, may have efficacy in treating cancers and particularly lymphoma.

Assay results for exemplary compounds are shown in Table 5.

TABLE 5
Z-138 Assay Results

| | IC50 Values |
|---|---|
| Compound | μMolar |
| 70 | 0.673 |
| 74 | 2.945 |
| 78 | 1.95 |
| 79 | 2.85 |
| 80 | 1.116 |
| 91 | 1.93 |
| 92 | 3.628 |
| 94 | 10.00 |

### Example 5: Mouse Pharmacokinetic Studies

Samples were obtained from 3 male BALB/c mice at staggered time points. Each mouse was given a tail bolus injection of 3 mg/kg injection of Compound 70 formulated as an IV solution with DMSO: PEG400: Aqueous solution with 20% HP-β-CD (5:30:65, v:v:v) (adjusted by 1N HCl). Blood samples (collected into lithium heparin tubes) were obtained at predose, and at 5, 15, 30 min and at 1, 2-, 4-, 8- and 24-hour, post injection. Plasma was separated from blood and stored at −20° C. prior to analysis. Samples were assayed by a LC/MS/MS procedure [AB Sciex API 4000; ACE Excel 5 C4 Column (50 mm*2.1 mm); Mobile Phase A: 5 mM NH4OAc (with 0.05% FA); Mobile Phase B: ACN (with 0.1% FA)]. Pharmacokinetic parameters were obtained from plasma concentrations using the R program—PK Package, Basic Non-Compartmental Pharmacokinetics, Thomas Jaki and Martin J. Wolfsegger Version 1.3-3, Jan. 6, 2016.

Assay results for compound 70 are shown in Table 6.

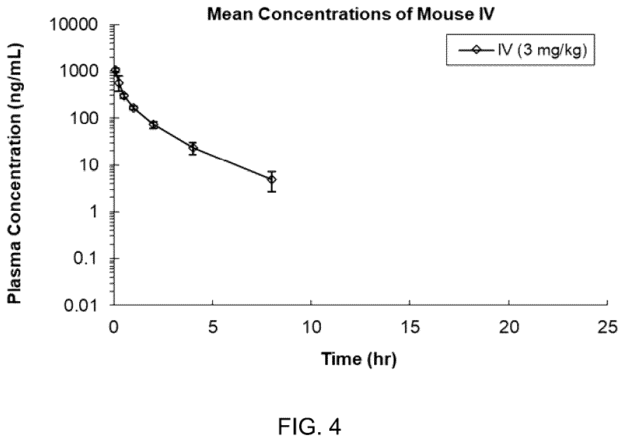

FIG. 4 shows a plot of the mouse PK data.

TABLE 6
Mouse PK Data

| Dose | t_{1/2} | C₀ | AUC_{Inf} | Vss | CL | MRT_{Inf} |
|---|---|---|---|---|---|---|
| (mg/kg) | (hr) | (ng/mL) | (hr*ng/mL) | (L/kg) | (mL/min/kg) | (hr) |
| 3 | 1.56 | 1523 | 748 | 4.88 | 66.9 | 1.22 |

### REFERENCES

1. Hwang, Jee Won, et al., Protein arginine methyltransferases: promising targets for cancer therapy. Experimental & Molecular Medicine (2021) 53: 788-808.

2. Lee, Michael K. C., et al., PRMT5: An Emerging Target for Pancreatic Adenocarcinoma, Cancers 2021, 13, 5136, doi.org/10.3390/cancers13205136.

3. Kim, Hyungsoo, et al., PRMT5 function and targeting in cancer, Cell Stress, August 2020, Vol. 4, No. 8, pp. 199-215.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### Equivalents

The invention can be embodied in other specific forms without departing from the spirit or essential characteristics thereof. The foregoing embodiments are to be considered in all respects illustrative rather than limiting on the invention described herein. In the various embodiments of the compositions and methods, where the term comprises is used with respect to the components of the compositions or the recited steps of the methods, it is also contemplated that the compositions and methods consist essentially of, or consist of, the recited components or steps. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, because upon mixing certain components can further react or be transformed into additional materials.

All percentages and ratios used herein, unless otherwise indicated, are by weight. It is recognized the mass of an object is often referred to as its weight in everyday usage and for most common scientific purposes, but that mass technically refers to the amount of matter of an object, whereas weight refers to the force experienced by an object due to gravity. Also, in common usage the “weight” (mass) of an object is what one determines when one “weighs” (masses) an object on a scale or balance.

What is claimed is:

1. A compound having the following formula 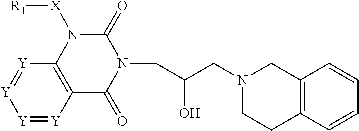
wherein
X is C₁-C₆ alkyl, CO, or a bond,
Y is, at each occurrence, is independently N, CH, or CR².
R¹ is H, C₁-C₆ alkyl, C₁-C₆ heteroalkyl, phenyl, C₃-C₇ heterocycle, C₃-C₇ carbocycle, C₅-C₆ heteroaromatic, C₈-C₁₁ spirocycle, C₈-C₁₁ heterospirocycles, amide, alkoxy, or C₇-C₁₂ fused or bridged bicyclics or heterobicyclics, optionally substituted at any position with one or more R³
R² is H, hydroxy, halo, C₁-C₆ alkyl halide, C₁-C₆ alkoxy, C₁-C₆ alkyl, C₁-C₆ alkenyl, C₁-C₆ alkynyl, C₁-C₆ heteroalkyl, C₁-C₆ heteroalkenyl, C₁-C₆ heteroalkynyl, amino, alkyl amide, alkyl carboxylic acid, pyridyl, methoxy pyridyl, phenyl, fluorophenyl, and cyano, or a pharmaceutically acceptable salt, ester, prodrug, or solvate thereof
wherein R¹ optionally has the formula A¹-L¹-B¹-, where A¹ is a first aromatic, heteroaromatic, carbocycle, heterocycle, spirocycle, or bridged or fused combinations thereof, where L¹ is a linker selected from alkyl, heteroalkyl, CO, O, N, or S, and where B¹ is a second aromatic, heteroaromatic, carbocycle, heterocycle, spirocycle, heterospirocycle, or bridged or fused combinations thereof, wherein B¹ is attached to the N of the remainder of the compound
wherein R³ is selected from H, halo, hydroxy, acyl, —C═OR⁴, alkyl halide, alkoxy, alkyl, alkenyl, alkynyl, heteroalkyl, heteroalkenyl, heteroalkynyl, amino, alkyl amide, alkyl carboxylic acid, and cyano, each optionally connected through a C₁-C₆ linker, and
wherein R⁴ is H or a C₁-C₆ alkyl, C₁-C₆ heteroalkyl, aryl, or heteroaryl, or a pharmaceuticaly acceptable salt, ester, prodrug, or solvate thereof.

2. A compound according to claim 1, wherein the compound is selected from one of the following compounds 1-105, or a pharmaceuticaly acceptable salt, ester, prodrug, or solvate thereof:

| | |
|---|---|
| 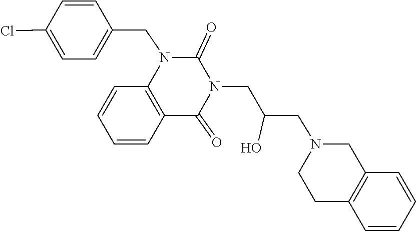 [SMILES: O=c1c2ccccc2n(Cc2ccc(Cl)cc2)c(=O)n1CC(O)CN1CCc2ccccc2C1] | 1 |
| 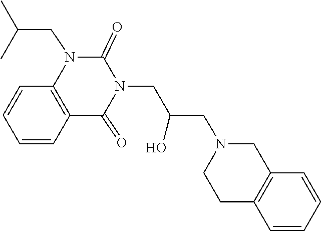 [SMILES: CC(C)Cn1c(=O)n(CC(O)CN2CCc3ccccc3C2)c(=O)c2ccccc21] | 2 |
| 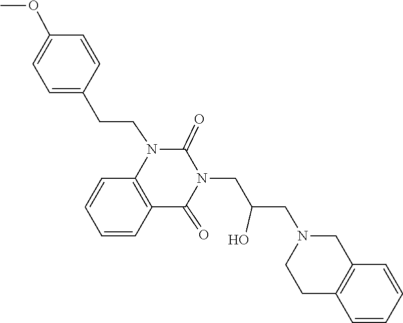 [SMILES: COc1ccc(CCn2c(=O)n(CC(O)CN3CCc4ccccc4C3)c(=O)c3ccccc32)cc1] | 3 |
| 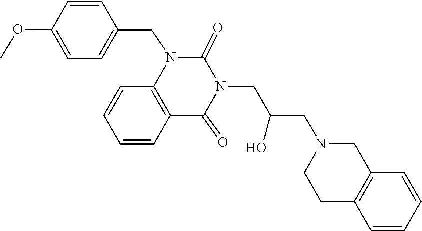 [SMILES: COc1ccc(Cn2c(=O)n(CC(O)CN3CCc4ccccc4C3)c(=O)c3ccccc32)cc1] | 4 |
| 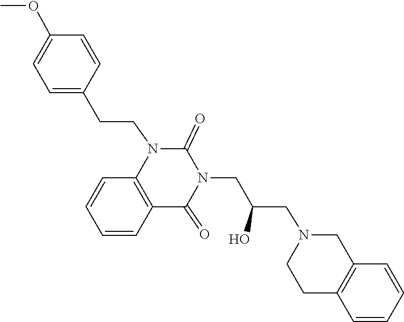 [SMILES: COc1ccc(CCn2c(=O)n(C[C@H](O)CN3CCc4ccccc4C3)c(=O)c3ccccc32)cc1] | 5 |
| 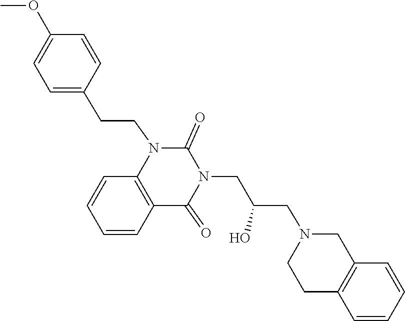 [SMILES: COc1ccc(CCn2c(=O)n(C[C@@H](O)CN3CCc4ccccc4C3)c(=O)c3ccccc32)cc1] | 6 |
| 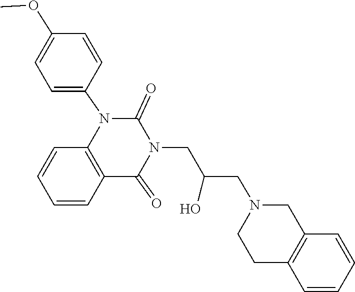 [SMILES: COc1ccc(-n2c(=O)n(CC(O)CN3CCc4ccccc4C3)c(=O)c3ccccc32)cc1] | 7 |
| 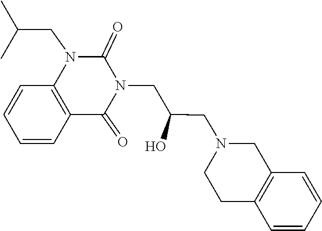 [SMILES: CC(C)Cn1c(=O)n(C[C@H](O)CN2CCc3ccccc3C2)c(=O)c2ccccc21] | 8 |
| 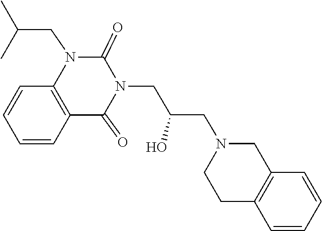 [SMILES: CC(C)Cn1c(=O)n(C[C@@H](O)CN2CCc3ccccc3C2)c(=O)c2ccccc21] | 9 |
| 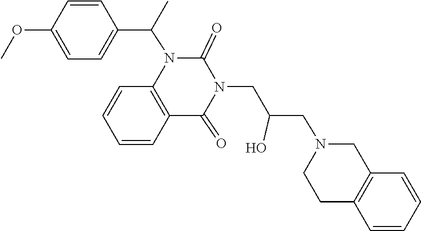 [SMILES: COc1ccc(C(C)n2c(=O)n(CC(O)CN3CCc4ccccc4C3)c(=O)c3ccccc32)cc1] | 10 |
| 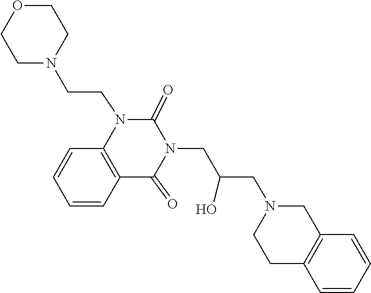 [SMILES: O=c1c2ccccc2n(CCN2CCOCC2)c(=O)n1CC(O)CN1CCc2ccccc2C1] | 11 |
| 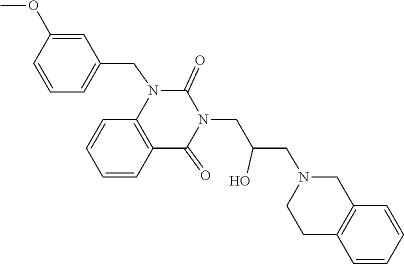 [SMILES: COc1cccc(Cn2c(=O)n(CC(O)CN3CCc4ccccc4C3)c(=O)c3ccccc32)c1] | 12 |
| 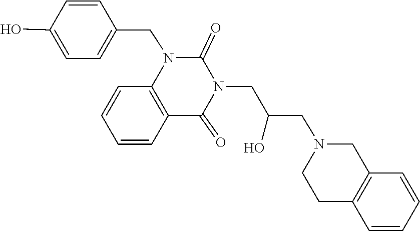 [SMILES: O=c1c2ccccc2n(Cc2ccc(O)cc2)c(=O)n1CC(O)CN1CCc2ccccc2C1] | 13 |
| 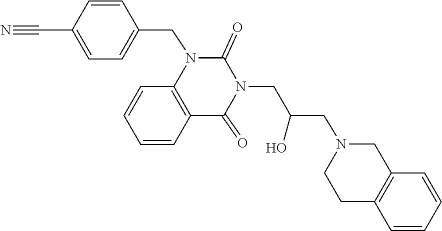 [SMILES: N#Cc1ccc(Cn2c(=O)n(CC(O)CN3CCc4ccccc4C3)c(=O)c3ccccc32)cc1] | 14 |
| 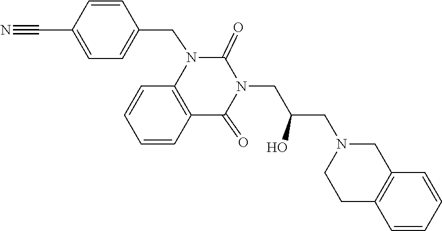 [SMILES: N#Cc1ccc(Cn2c(=O)n(C[C@H](O)CN3CCc4ccccc4C3)c(=O)c3ccccc32)cc1] | 15 |
| 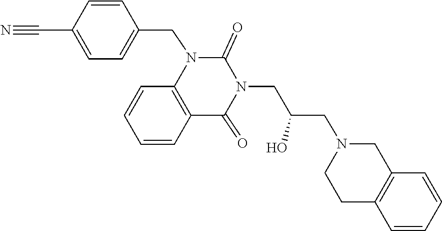 [SMILES: N#Cc1ccc(Cn2c(=O)n(C[C@@H](O)CN3CCc4ccccc4C3)c(=O)c3ccccc32)cc1] | 16 |
| 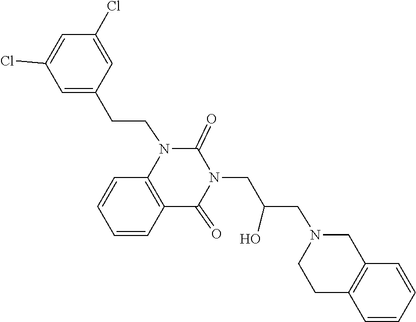 [SMILES: O=c1c2ccccc2n(CCc2cc(Cl)cc(Cl)c2)c(=O)n1CC(O)CN1CCc2ccccc2C1] | 17 |
| 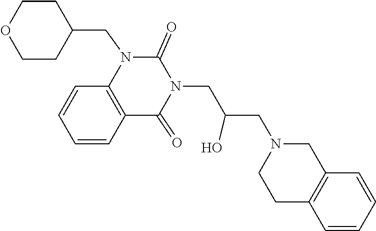 [SMILES: O=c1c2ccccc2n(CC2CCOCC2)c(=O)n1CC(O)CN1CCc2ccccc2C1] | 18 |
| 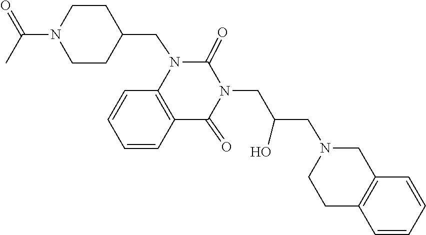 [SMILES: CC(=O)N1CCC(Cn2c(=O)n(CC(O)CN3CCc4ccccc4C3)c(=O)c3ccccc32)CC1] | 19 |
| 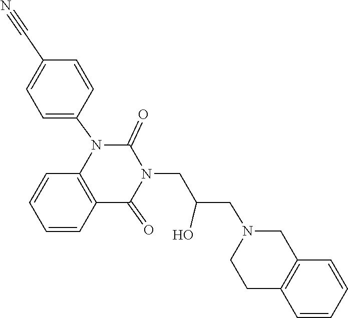 [SMILES: N#Cc1ccc(-n2c(=O)n(CC(O)CN3CCc4ccccc4C3)c(=O)c3ccccc32)cc1] | 20 |
| 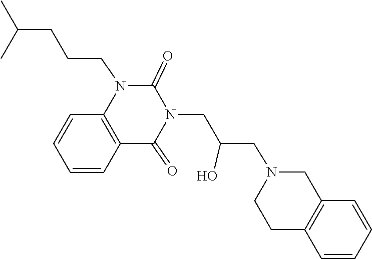 [SMILES: CC(C)CCCn1c(=O)n(CC(O)CN2CCc3ccccc3C2)c(=O)c2ccccc21] | 21 |
| 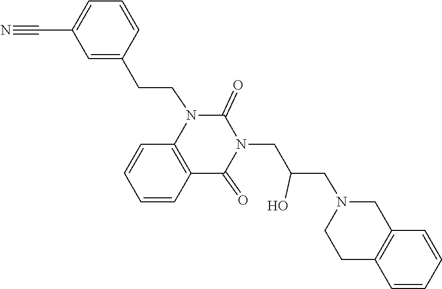 [SMILES: N#Cc1cccc(CCn2c(=O)n(CC(O)CN3CCc4ccccc4C3)c(=O)c3ccccc32)c1] | 22 |
| 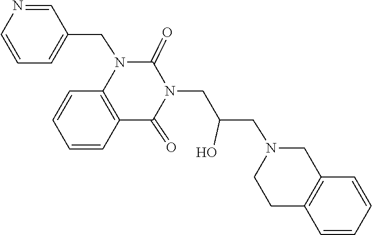 [SMILES: O=c1c2ccccc2n(Cc2cccnc2)c(=O)n1CC(O)CN1CCc2ccccc2C1] | 23 |
| 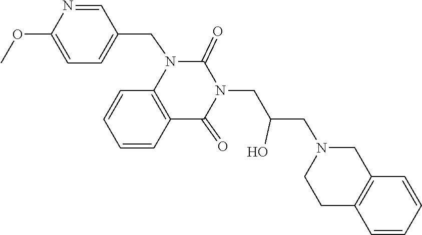 [SMILES: COc1ccc(Cn2c(=O)n(CC(O)CN3CCc4ccccc4C3)c(=O)c3ccccc32)cn1] | 24 |
| 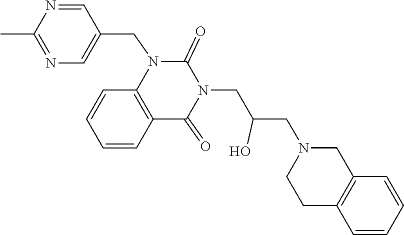 [SMILES: Cc1ncc(Cn2c(=O)n(CC(O)CN3CCc4ccccc4C3)c(=O)c3ccccc32)cn1] | 25 |
| 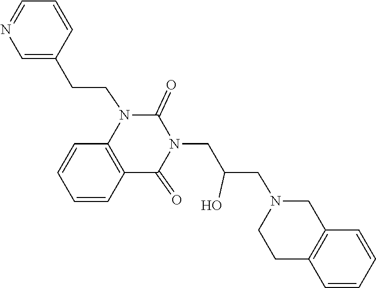 [SMILES: O=c1c2ccccc2n(CCc2cccnc2)c(=O)n1CC(O)CN1CCc2ccccc2C1] | 26 |
| 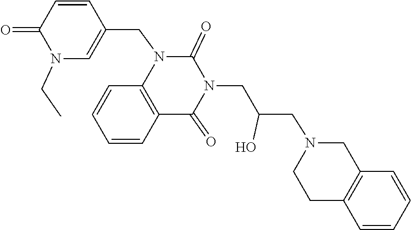 [SMILES: CCn1cc(Cn2c(=O)n(CC(O)CN3CCc4ccccc4C3)c(=O)c3ccccc32)ccc1=O] | 27 |
| 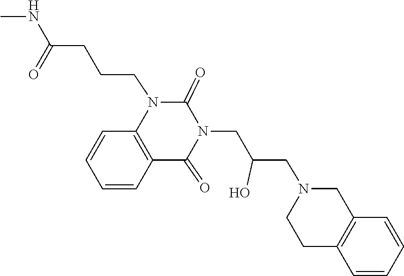 [SMILES: CNC(=O)CCCn1c(=O)n(CC(O)CN2CCc3ccccc3C2)c(=O)c2ccccc21] | 28 |
| 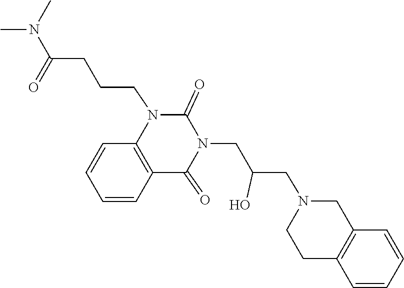 [SMILES: CN(C)C(=O)CCCn1c(=O)n(CC(O)CN2CCc3ccccc3C2)c(=O)c2ccccc21] | 29 |
| 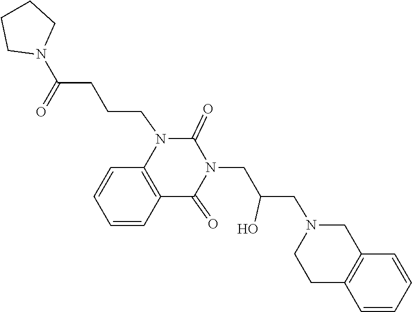 [SMILES: O=C(CCCn1c(=O)n(CC(O)CN2CCc3ccccc3C2)c(=O)c2ccccc21)N1CCCC1] | 30 |
| 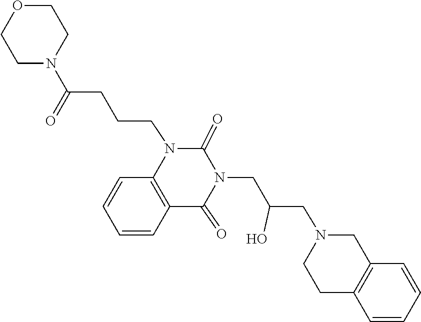 [SMILES: O=C(CCCn1c(=O)n(CC(O)CN2CCc3ccccc3C2)c(=O)c2ccccc21)N1CCOCC1] | 31 |
| 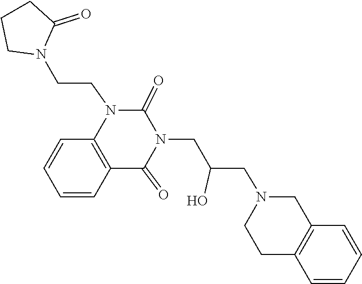 [SMILES: O=C1CCCN1CCn1c(=O)n(CC(O)CN2CCc3ccccc3C2)c(=O)c2ccccc21] | 32 |
| 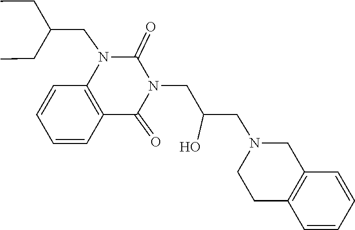 [SMILES: CCC(CC)Cn1c(=O)n(CC(O)CN2CCc3ccccc3C2)c(=O)c2ccccc21] | 33 |
| 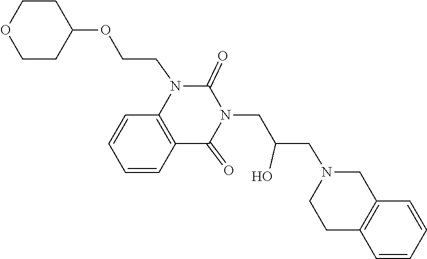 [SMILES: O=c1c2ccccc2n(CCOC2CCOCC2)c(=O)n1CC(O)CN1CCc2ccccc2C1] | 34 |
| 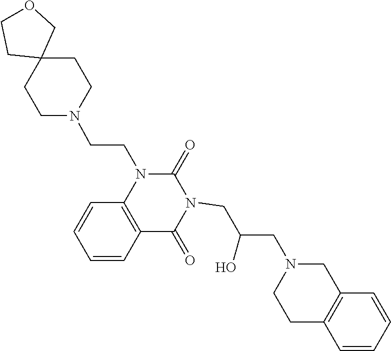 [SMILES: O=c1c2ccccc2n(CCN2CCC3(CCOC3)CC2)c(=O)n1CC(O)CN1CCc2ccccc2C1] | 35 |
| 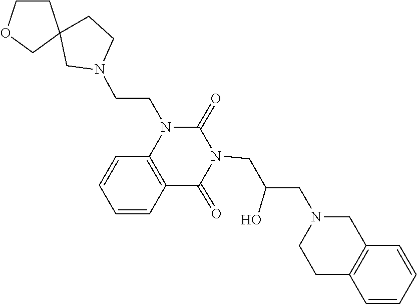 [SMILES: O=c1c2ccccc2n(CCN2CCC3(CCOC3)C2)c(=O)n1CC(O)CN1CCc2ccccc2C1] | 36 |
| 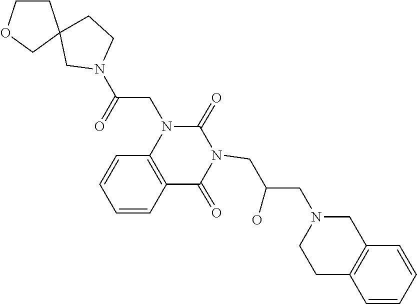 | 37 |
| 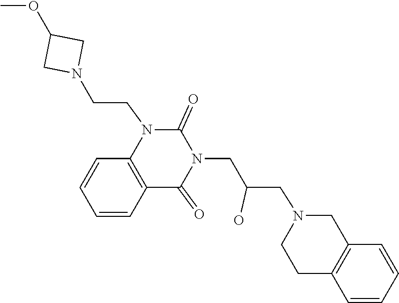 | 38 |
| 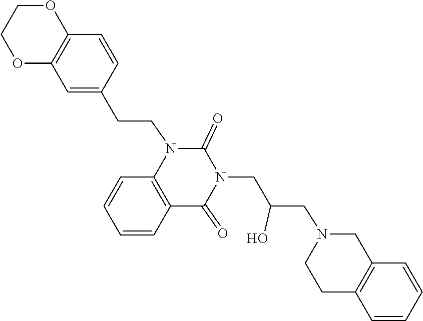 [SMILES: O=c1c2ccccc2n(CCc2ccc3c(c2)OCCO3)c(=O)n1CC(O)CN1CCc2ccccc2C1] | 39 |
| 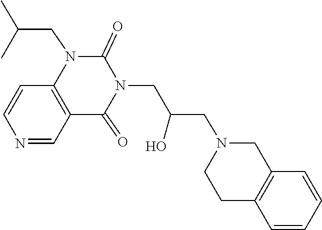 [SMILES: CC(C)Cn1c(=O)n(CC(O)CN2CCc3ccccc3C2)c(=O)c2cnccc21] | 40 |
| 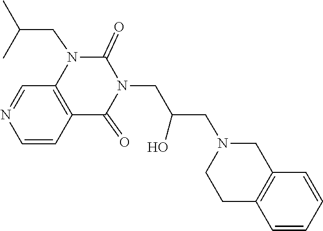 [SMILES: CC(C)Cn1c(=O)n(CC(O)CN2CCc3ccccc3C2)c(=O)c2ccncc21] | 41 |
| 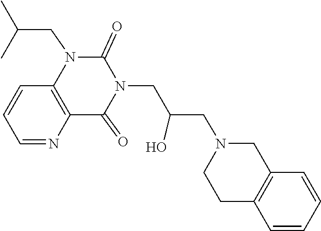 [SMILES: CC(C)Cn1c(=O)n(CC(O)CN2CCc3ccccc3C2)c(=O)c2ncccc21] | 42 |
| 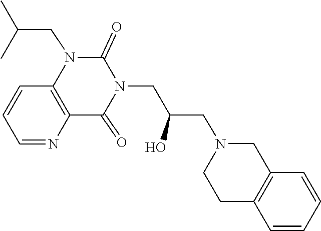 [SMILES: CC(C)Cn1c(=O)n(C[C@H](O)CN2CCc3ccccc3C2)c(=O)c2ncccc21] | 43 |
| 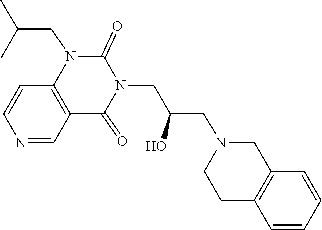 [SMILES: CC(C)Cn1c(=O)n(C[C@H](O)CN2CCc3ccccc3C2)c(=O)c2cnccc21] | 44 |
| 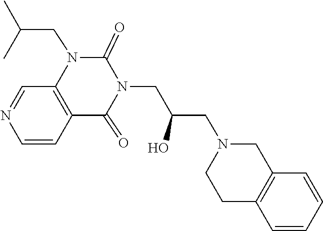 [SMILES: CC(C)Cn1c(=O)n(C[C@H](O)CN2CCc3ccccc3C2)c(=O)c2ccncc21] | 45 |
| 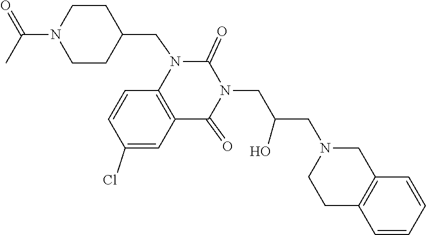 [SMILES: CC(=O)N1CCC(Cn2c(=O)n(CC(O)CN3CCc4ccccc4C3)c(=O)c3cc(Cl)ccc32)CC1] | 46 |
| 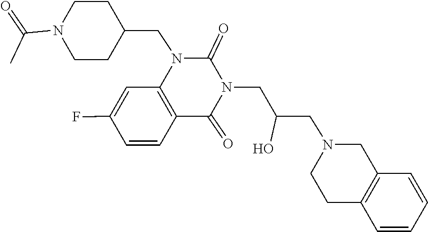 [SMILES: CC(=O)N1CCC(Cn2c(=O)n(CC(O)CN3CCc4ccccc4C3)c(=O)c3ccc(F)cc32)CC1] | 47 |
| 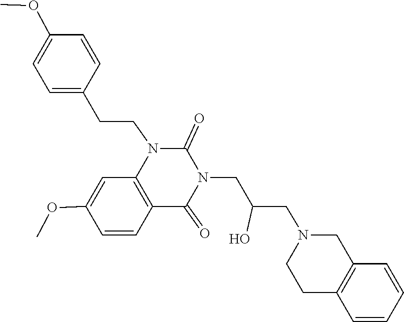 [SMILES: COc1ccc(CCn2c(=O)n(CC(O)CN3CCc4ccccc4C3)c(=O)c3ccc(OC)cc32)cc1] | 48 |
| 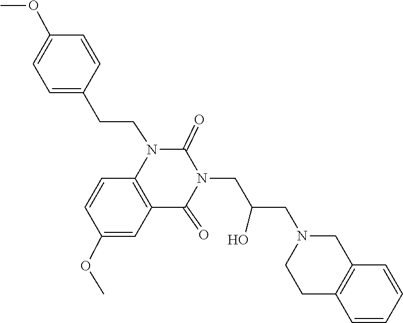 [SMILES: COc1ccc(CCn2c(=O)n(CC(O)CN3CCc4ccccc4C3)c(=O)c3cc(OC)ccc32)cc1] | 49 |
| 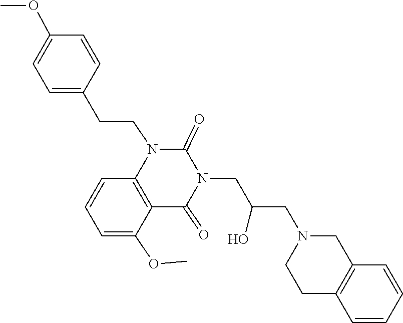 [SMILES: COc1ccc(CCn2c(=O)n(CC(O)CN3CCc4ccccc4C3)c(=O)c3c(OC)cccc32)cc1] | 50 |
| 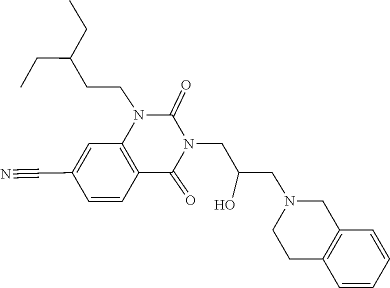 [SMILES: CCC(CC)CCn1c(=O)n(CC(O)CN2CCc3ccccc3C2)c(=O)c2ccc(C#N)cc21] | 51 |
| 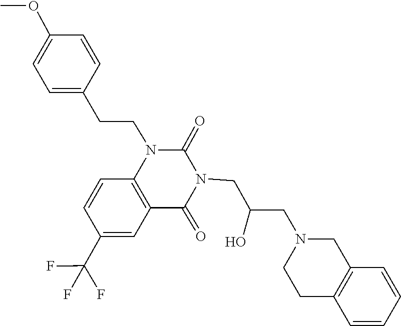 [SMILES: COc1ccc(CCn2c(=O)n(CC(O)CN3CCc4ccccc4C3)c(=O)c3cc(C(F)(F)F)ccc32)cc1] | 52 |
| 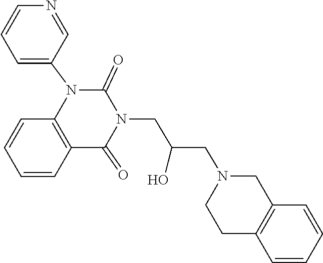 [SMILES: O=c1c2ccccc2n(-c2cccnc2)c(=O)n1CC(O)CN1CCc2ccccc2C1] | 53 |
| 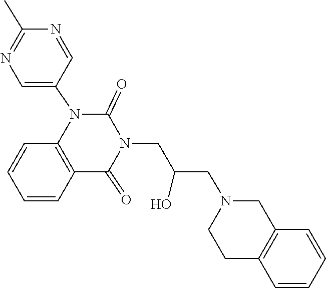 [SMILES: Cc1ncc(-n2c(=O)n(CC(O)CN3CCc4ccccc4C3)c(=O)c3ccccc32)cn1] | 54 |
| 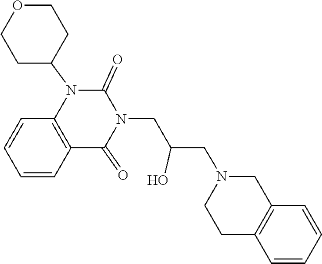 [SMILES: O=c1c2ccccc2n(C2CCOCC2)c(=O)n1CC(O)CN1CCc2ccccc2C1] | 55 |
| 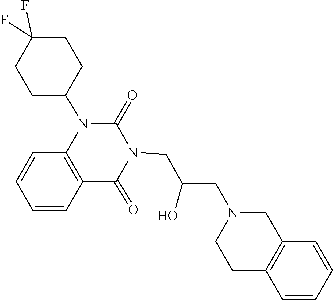 [SMILES: O=c1c2ccccc2n(C2CCC(F)(F)CC2)c(=O)n1CC(O)CN1CCc2ccccc2C1] | 56 |
| 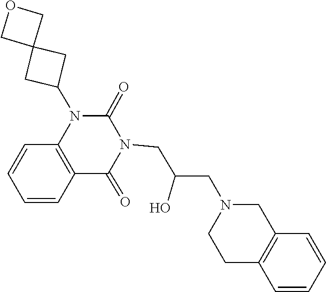 [SMILES: O=c1c2ccccc2n(C2CC3(COC3)C2)c(=O)n1CC(O)CN1CCc2ccccc2C1] | 57 |
| 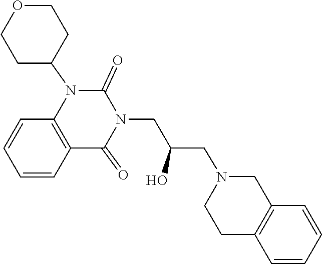 [SMILES: O=c1c2ccccc2n(C2CCOCC2)c(=O)n1C[C@H](O)CN1CCc2ccccc2C1] | 58 |
| 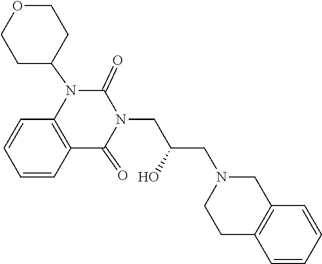 [SMILES: O=c1c2ccccc2n(C2CCOCC2)c(=O)n1C[C@@H](O)CN1CCc2ccccc2C1] | 59 |
| 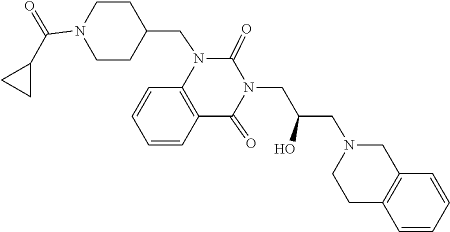 [SMILES: O=C(C1CC1)N1CCC(Cn2c(=O)n(C[C@H](O)CN3CCc4ccccc4C3)c(=O)c3ccccc32)CC1] | 60 |
| 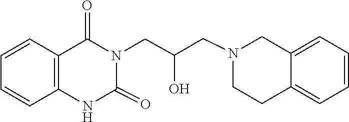 [SMILES: O=c1[nH]c2ccccc2c(=O)n1CC(O)CN1CCc2ccccc2C1] | 61 |
| 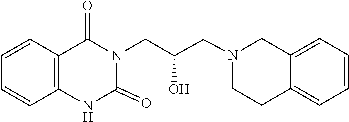 [SMILES: O=c1[nH]c2ccccc2c(=O)n1C[C@@H](O)CN1CCc2ccccc2C1] | 62 |
| 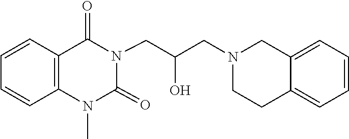 [SMILES: Cn1c(=O)n(CC(O)CN2CCc3ccccc3C2)c(=O)c2ccccc21] | 63 |
| 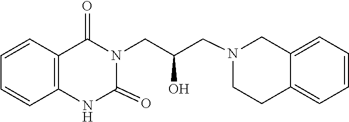 [SMILES: O=c1[nH]c2ccccc2c(=O)n1C[C@H](O)CN1CCc2ccccc2C1] | 64 |
| 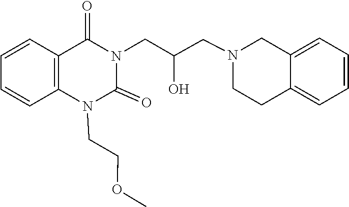 [SMILES: COCCn1c(=O)n(CC(O)CN2CCc3ccccc3C2)c(=O)c2ccccc21] | 65 |
| 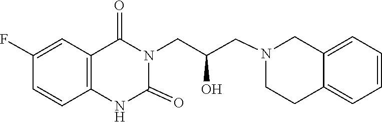 [SMILES: O=c1[nH]c2ccc(F)cc2c(=O)n1C[C@H](O)CN1CCc2ccccc2C1] | 66 |
| 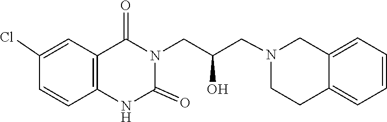 [SMILES: O=c1[nH]c2ccc(Cl)cc2c(=O)n1C[C@H](O)CN1CCc2ccccc2C1] | 67 |
| 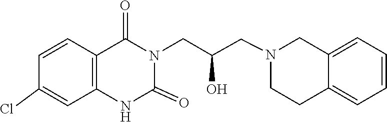 [SMILES: O=c1[nH]c2cc(Cl)ccc2c(=O)n1C[C@H](O)CN1CCc2ccccc2C1] | 68 |
| 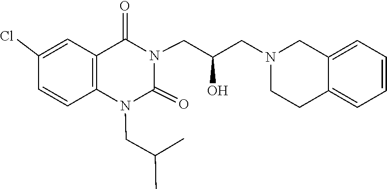 [SMILES: CC(C)Cn1c(=O)n(C[C@H](O)CN2CCc3ccccc3C2)c(=O)c2cc(Cl)ccc21] | 69 |
| 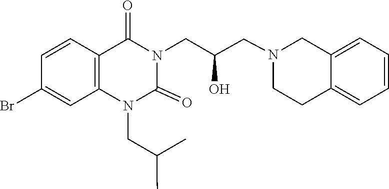 [SMILES: CC(C)Cn1c(=O)n(C[C@H](O)CN2CCc3ccccc3C2)c(=O)c2ccc(Br)cc21] | 70 |
| 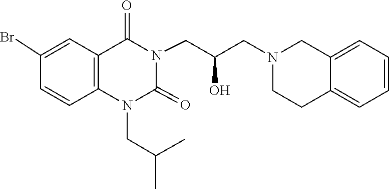 [SMILES: CC(C)Cn1c(=O)n(C[C@H](O)CN2CCc3ccccc3C2)c(=O)c2cc(Br)ccc21] | 71 |
| 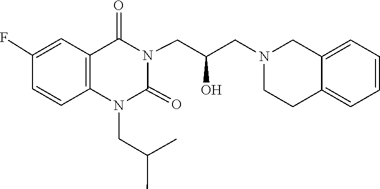 [SMILES: CC(C)Cn1c(=O)n(C[C@H](O)CN2CCc3ccccc3C2)c(=O)c2cc(F)ccc21] | 72 |
| 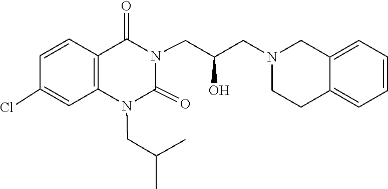 [SMILES: CC(C)Cn1c(=O)n(C[C@H](O)CN2CCc3ccccc3C2)c(=O)c2ccc(Cl)cc21] | 73 |
| 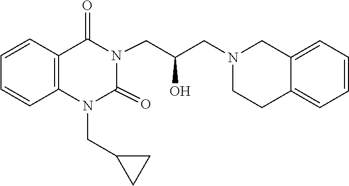 [SMILES: O=c1c2ccccc2n(CC2CC2)c(=O)n1C[C@H](O)CN1CCc2ccccc2C1] | 74 |
| 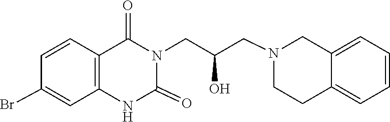 [SMILES: O=c1[nH]c2cc(Br)ccc2c(=O)n1C[C@H](O)CN1CCc2ccccc2C1] | 75 |
| 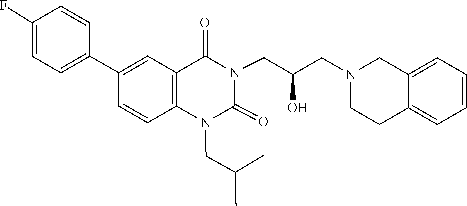 [SMILES: CC(C)Cn1c(=O)n(C[C@H](O)CN2CCc3ccccc3C2)c(=O)c2cc(-c3ccc(F)cc3)ccc21] | 76 |
| 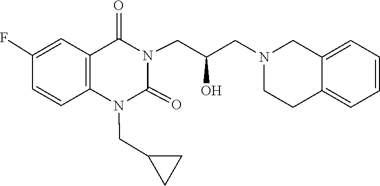 [SMILES: O=c1c2cc(F)ccc2n(CC2CC2)c(=O)n1C[C@H](O)CN1CCc2ccccc2C1] | 77 |
| 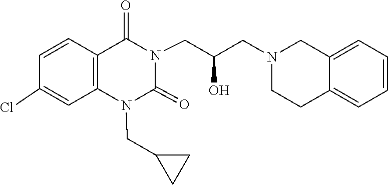 [SMILES: O=c1c2ccc(Cl)cc2n(CC2CC2)c(=O)n1C[C@H](O)CN1CCc2ccccc2C1] | 78 |
| 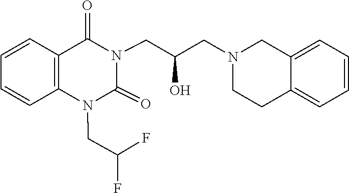 [SMILES: O=c1c2ccccc2n(CC(F)F)c(=O)n1C[C@H](O)CN1CCc2ccccc2C1] | 79 |
| 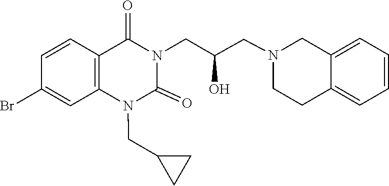 [SMILES: O=c1c2ccc(Br)cc2n(CC2CC2)c(=O)n1C[C@H](O)CN1CCc2ccccc2C1] | 80 |
| 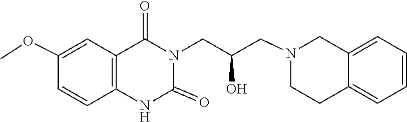 [SMILES: COc1ccc2[nH]c(=O)n(C[C@H](O)CN3CCc4ccccc4C3)c(=O)c2c1] | 81 |
| 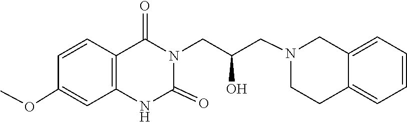 [SMILES: COc1ccc2c(=O)n(C[C@H](O)CN3CCc4ccccc4C3)c(=O)[nH]c2c1] | 82 |
| 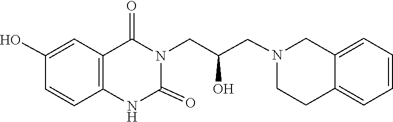 [SMILES: O=c1[nH]c2ccc(O)cc2c(=O)n1C[C@H](O)CN1CCc2ccccc2C1] | 83 |
| 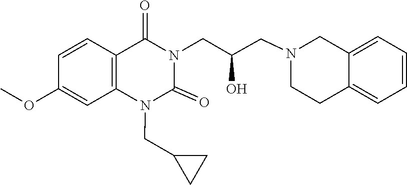 [SMILES: COc1ccc2c(=O)n(C[C@H](O)CN3CCc4ccccc4C3)c(=O)n(CC3CC3)c2c1] | 84 |
| 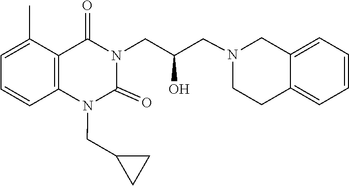 [SMILES: Cc1cccc2c1c(=O)n(C[C@H](O)CN1CCc3ccccc3C1)c(=O)n2CC1CC1] | 85 |
| 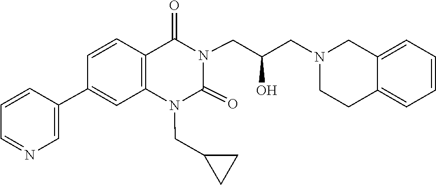 [SMILES: O=c1c2ccc(-c3cccnc3)cc2n(CC2CC2)c(=O)n1C[C@H](O)CN1CCc2ccccc2C1] | 86 |
| 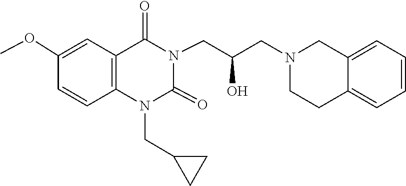 [SMILES: COc1ccc2c(c1)c(=O)n(C[C@H](O)CN1CCc3ccccc3C1)c(=O)n2CC1CC1] | 87 |
| 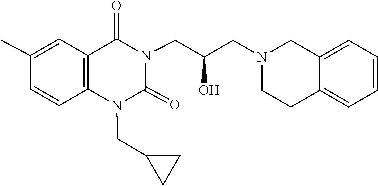 [SMILES: Cc1ccc2c(c1)c(=O)n(C[C@H](O)CN1CCc3ccccc3C1)c(=O)n2CC1CC1] | 88 |
| 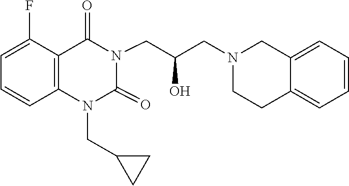 [SMILES: O=c1c2c(F)cccc2n(CC2CC2)c(=O)n1C[C@H](O)CN1CCc2ccccc2C1] | 89 |
| 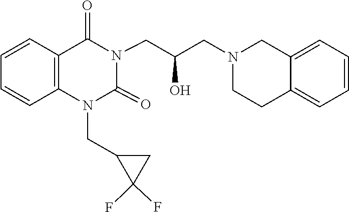 [SMILES: O=c1c2ccccc2n(CC2CC2(F)F)c(=O)n1C[C@H](O)CN1CCc2ccccc2C1] | 90 |
| 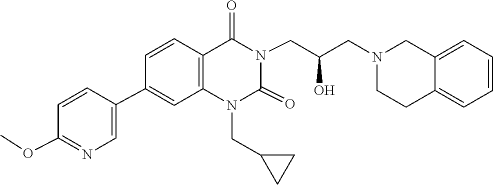 [SMILES: COc1ccc(-c2ccc3c(=O)n(C[C@H](O)CN4CCc5ccccc5C4)c(=O)n(CC4CC4)c3c2)cn1] | 91 |
| 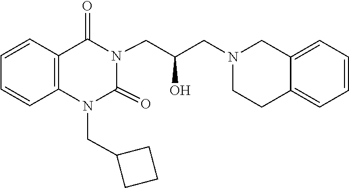 [SMILES: O=c1c2ccccc2n(CC2CCC2)c(=O)n1C[C@H](O)CN1CCc2ccccc2C1] | 92 |
| 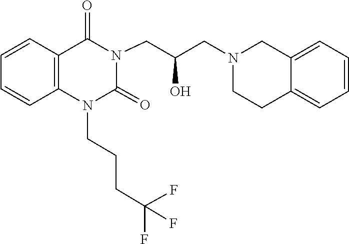 [SMILES: O=c1c2ccccc2n(CCCC(F)(F)F)c(=O)n1C[C@H](O)CN1CCc2ccccc2C1] | 93 |
| 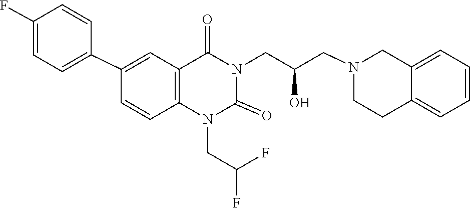 [SMILES: O=c1c2cc(-c3ccc(F)cc3)ccc2n(CC(F)F)c(=O)n1C[C@H](O)CN1CCc2ccccc2C1] | 94 |
| 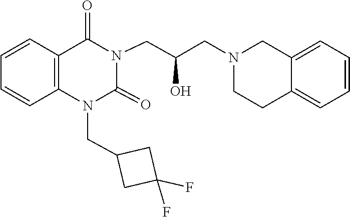 [SMILES: O=c1c2ccccc2n(CC2CC(F)(F)C2)c(=O)n1C[C@H](O)CN1CCc2ccccc2C1] | 95 |
| 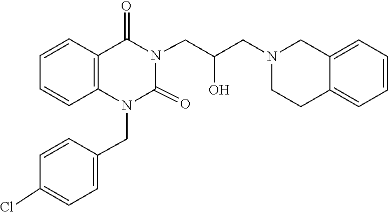 [SMILES: O=c1c2ccccc2n(Cc2ccc(Cl)cc2)c(=O)n1CC(O)CN1CCc2ccccc2C1] | 96 |
| 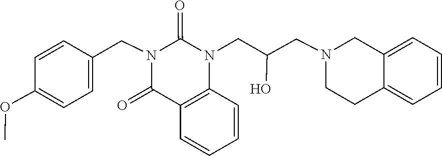 [SMILES: COc1ccc(Cn2c(=O)c3ccccc3n(CC(O)CN3CCc4ccccc4C3)c2=O)cc1] | 97 |
| 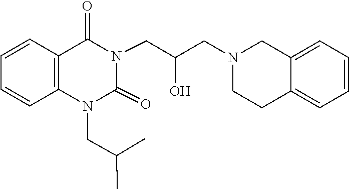 [SMILES: CC(C)Cn1c(=O)n(CC(O)CN2CCc3ccccc3C2)c(=O)c2ccccc21] | 98 |
| 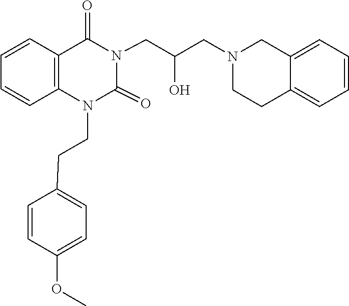 [SMILES: COc1ccc(CCn2c(=O)n(CC(O)CN3CCc4ccccc4C3)c(=O)c3ccccc32)cc1] | 99 |
| 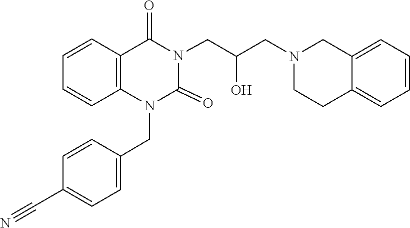 [SMILES: N#Cc1ccc(Cn2c(=O)n(CC(O)CN3CCc4ccccc4C3)c(=O)c3ccccc32)cc1] | 100 |
| 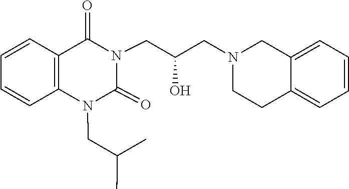 [SMILES: CC(C)Cn1c(=O)n(C[C@@H](O)CN2CCc3ccccc3C2)c(=O)c2ccccc21] | 101 |
| 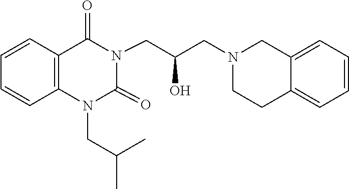 [SMILES: CC(C)Cn1c(=O)n(C[C@H](O)CN2CCc3ccccc3C2)c(=O)c2ccccc21] | 102 |
| 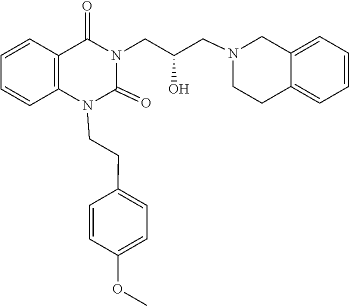 [SMILES: COc1ccc(CCn2c(=O)n(C[C@@H](O)CN3CCc4ccccc4C3)c(=O)c3ccccc32)cc1] | 103 |
| 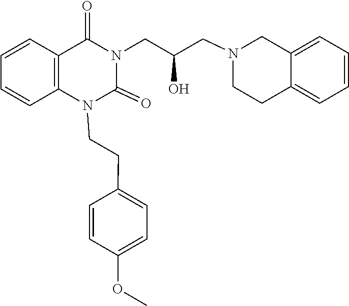 [SMILES: COc1ccc(CCn2c(=O)n(C[C@H](O)CN3CCc4ccccc4C3)c(=O)c3ccccc32)cc1] | 104 |
| 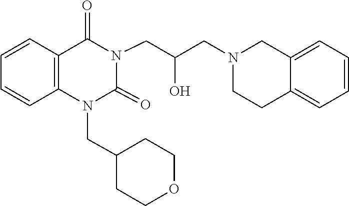 [SMILES: O=c1c2ccccc2n(CC2CCOCC2)c(=O)n1CC(O)CN1CCc2ccccc2C1] | 105 |

3. A compound selected from one of the following compounds 1-105, or a pharmaceutically acceptable salt, ester, prodrug, or solvate thereof:

| | |
|---|---|
| 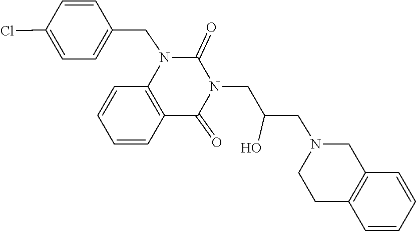 [SMILES: O=c1c2ccccc2n(Cc2ccc(Cl)cc2)c(=O)n1CC(O)CN1CCc2ccccc2C1] | 1 |
| 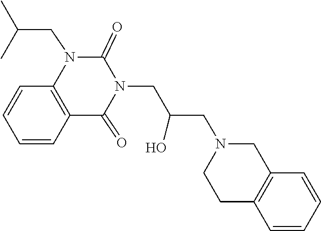 [SMILES: CC(C)Cn1c(=O)n(CC(O)CN2CCc3ccccc3C2)c(=O)c2ccccc21] | 2 |
| 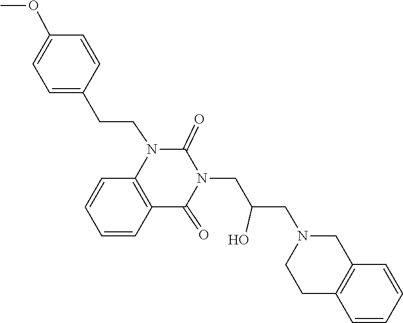 [SMILES: COc1ccc(CCn2c(=O)n(CC(O)CN3CCc4ccccc4C3)c(=O)c3ccccc32)cc1] | 3 |
| 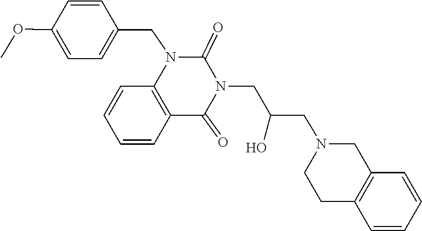 [SMILES: COc1ccc(Cn2c(=O)n(CC(O)CN3CCc4ccccc4C3)c(=O)c3ccccc32)cc1] | 4 |
| 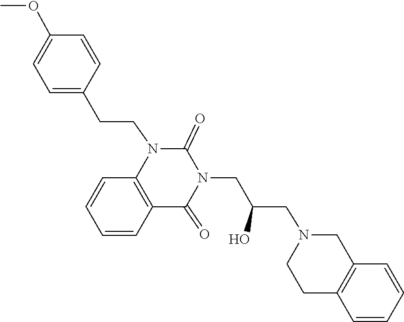 [SMILES: COc1ccc(CCn2c(=O)n(C[C@H](O)CN3CCc4ccccc4C3)c(=O)c3ccccc32)cc1] | 5 |
| 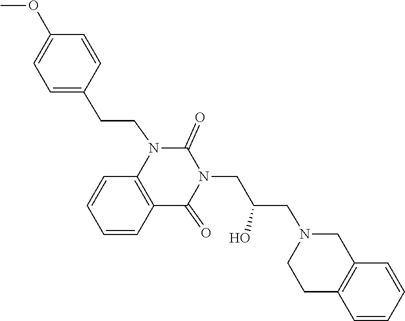 [SMILES: COc1ccc(CCn2c(=O)n(C[C@@H](O)CN3CCc4ccccc4C3)c(=O)c3ccccc32)cc1] | 6 |
| 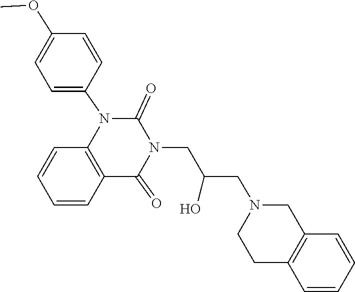 [SMILES: COc1ccc(-n2c(=O)n(CC(O)CN3CCc4ccccc4C3)c(=O)c3ccccc32)cc1] | 7 |
| 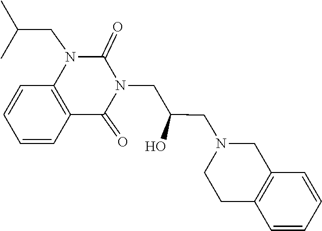 [SMILES: CC(C)Cn1c(=O)n(C[C@H](O)CN2CCc3ccccc3C2)c(=O)c2ccccc21] | 8 |
| 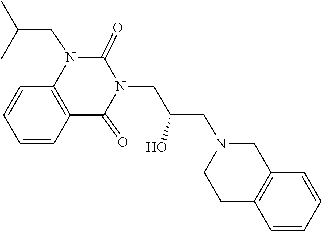 [SMILES: CC(C)Cn1c(=O)n(C[C@@H](O)CN2CCc3ccccc3C2)c(=O)c2ccccc21] | 9 |
| 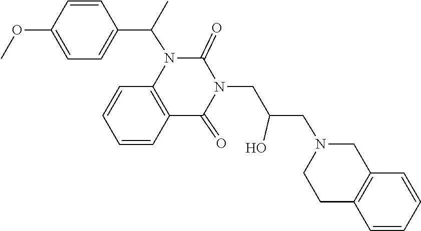 [SMILES: COc1ccc(C(C)n2c(=O)n(CC(O)CN3CCc4ccccc4C3)c(=O)c3ccccc32)cc1] | 10 |
| 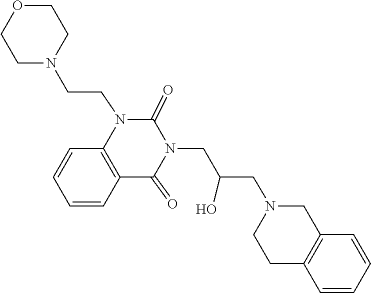 [SMILES: O=c1c2ccccc2n(CCN2CCOCC2)c(=O)n1CC(O)CN1CCc2ccccc2C1] | 11 |
| 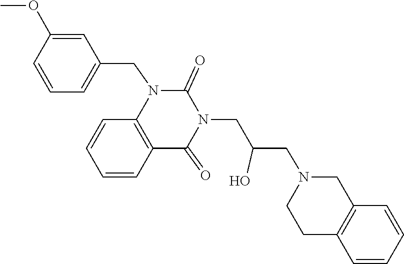 [SMILES: COc1cccc(Cn2c(=O)n(CC(O)CN3CCc4ccccc4C3)c(=O)c3ccccc32)c1] | 12 |
| 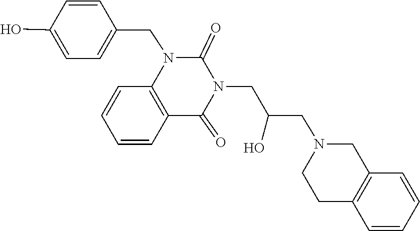 [SMILES: O=c1c2ccccc2n(Cc2ccc(O)cc2)c(=O)n1CC(O)CN1CCc2ccccc2C1] | 13 |
| 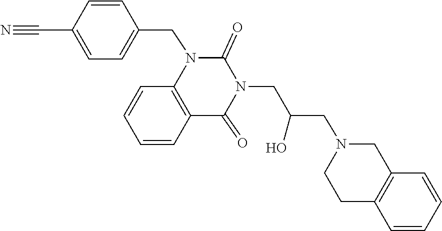 [SMILES: N#Cc1ccc(Cn2c(=O)n(CC(O)CN3CCc4ccccc4C3)c(=O)c3ccccc32)cc1] | 14 |
| 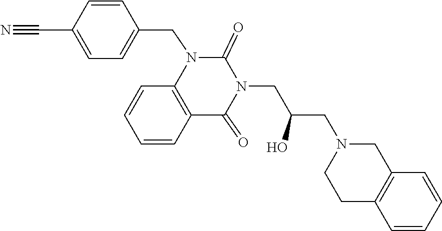 [SMILES: N#Cc1ccc(Cn2c(=O)n(C[C@H](O)CN3CCc4ccccc4C3)c(=O)c3ccccc32)cc1] | 15 |
| 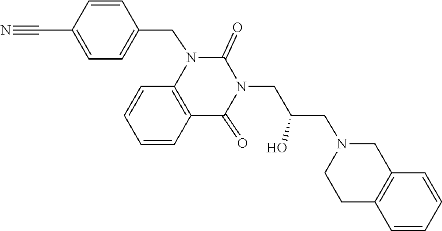 [SMILES: N#Cc1ccc(Cn2c(=O)n(C[C@@H](O)CN3CCc4ccccc4C3)c(=O)c3ccccc32)cc1] | 16 |
| 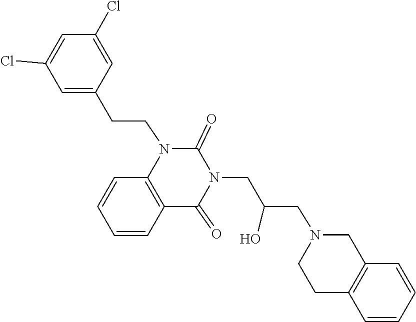 [SMILES: O=c1c2ccccc2n(CCc2cc(Cl)cc(Cl)c2)c(=O)n1CC(O)CN1CCc2ccccc2C1] | 17 |
| 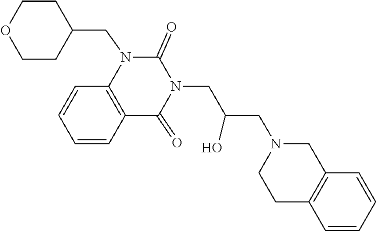 [SMILES: O=c1c2ccccc2n(CC2CCOCC2)c(=O)n1CC(O)CN1CCc2ccccc2C1] | 18 |
| 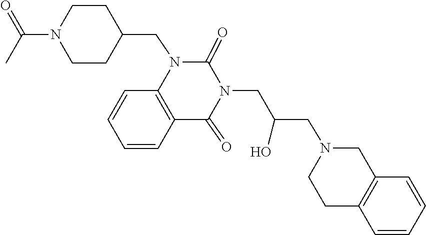 [SMILES: CC(=O)N1CCC(Cn2c(=O)n(CC(O)CN3CCc4ccccc4C3)c(=O)c3ccccc32)CC1] | 19 |
| 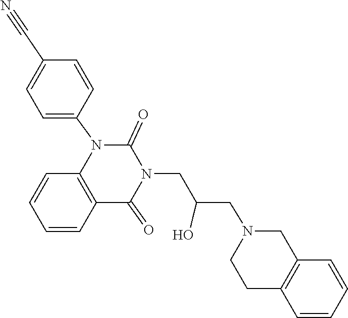 [SMILES: N#Cc1ccc(-n2c(=O)n(CC(O)CN3CCc4ccccc4C3)c(=O)c3ccccc32)cc1] | 20 |
| 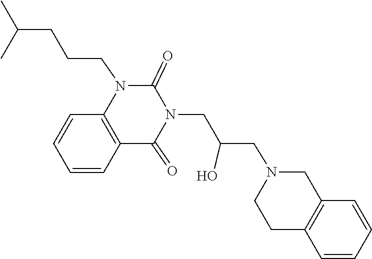 [SMILES: CC(C)CCCn1c(=O)n(CC(O)CN2CCc3ccccc3C2)c(=O)c2ccccc21] | 21 |
| 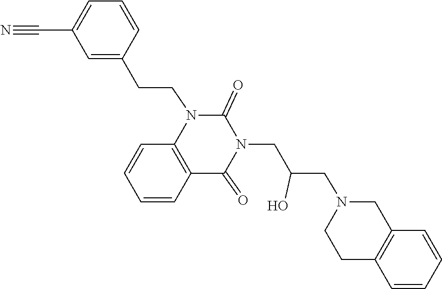 [SMILES: N#Cc1cccc(CCn2c(=O)n(CC(O)CN3CCc4ccccc4C3)c(=O)c3ccccc32)c1] | 22 |
| 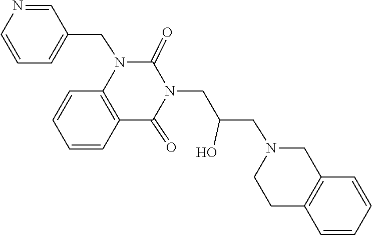 [SMILES: O=c1c2ccccc2n(Cc2cccnc2)c(=O)n1CC(O)CN1CCc2ccccc2C1] | 23 |
| 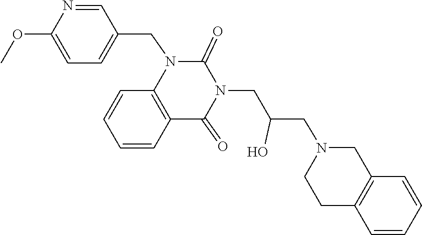 [SMILES: COc1ccc(Cn2c(=O)n(CC(O)CN3CCc4ccccc4C3)c(=O)c3ccccc32)cn1] | 24 |
| 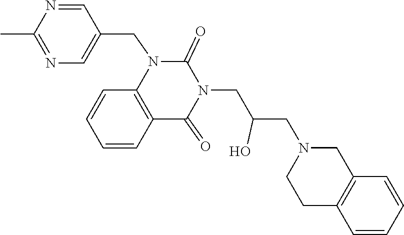 [SMILES: Cc1ncc(Cn2c(=O)n(CC(O)CN3CCc4ccccc4C3)c(=O)c3ccccc32)cn1] | 25 |
| 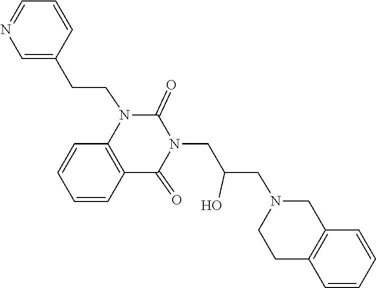 [SMILES: O=c1c2ccccc2n(CCc2cccnc2)c(=O)n1CC(O)CN1CCc2ccccc2C1] | 26 |
| 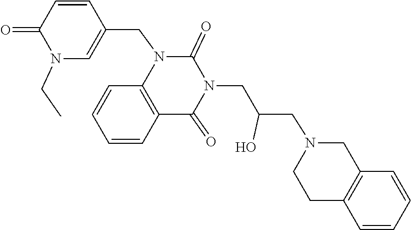 [SMILES: CCn1cc(Cn2c(=O)n(CC(O)CN3CCc4ccccc4C3)c(=O)c3ccccc32)ccc1=O] | 27 |
| 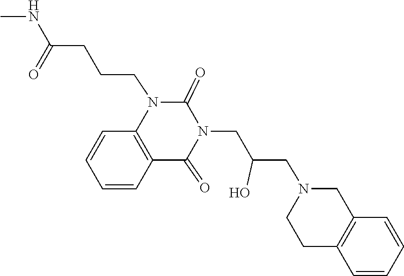 [SMILES: CNC(=O)CCCn1c(=O)n(CC(O)CN2CCc3ccccc3C2)c(=O)c2ccccc21] | 28 |
| 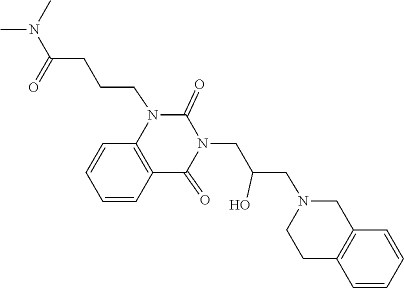 [SMILES: CN(C)C(=O)CCCn1c(=O)n(CC(O)CN2CCc3ccccc3C2)c(=O)c2ccccc21] | 29 |
| 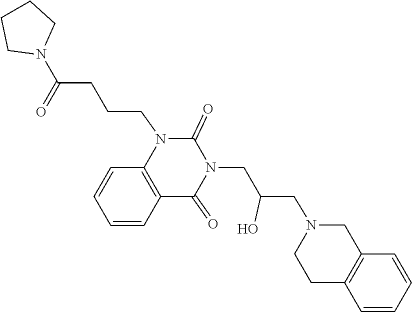 [SMILES: O=C(CCCn1c(=O)n(CC(O)CN2CCc3ccccc3C2)c(=O)c2ccccc21)N1CCCC1] | 30 |
| 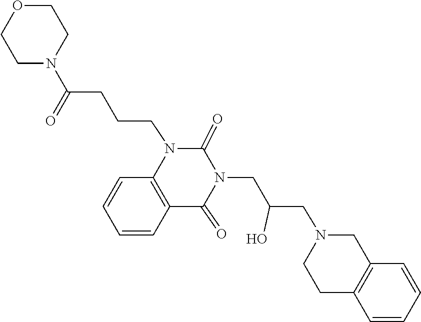 [SMILES: O=C(CCCn1c(=O)n(CC(O)CN2CCc3ccccc3C2)c(=O)c2ccccc21)N1CCOCC1] | 31 |
| 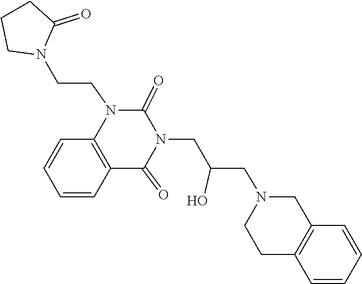 [SMILES: O=C1CCCN1CCn1c(=O)n(CC(O)CN2CCc3ccccc3C2)c(=O)c2ccccc21] | 32 |
| 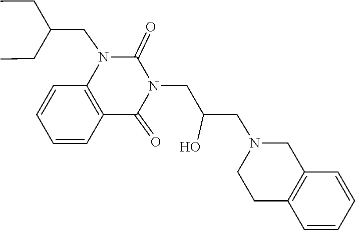 [SMILES: CCC(CC)Cn1c(=O)n(CC(O)CN2CCc3ccccc3C2)c(=O)c2ccccc21] | 33 |
| 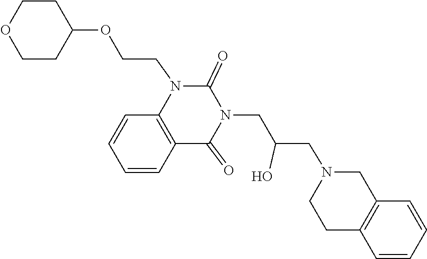 [SMILES: O=c1c2ccccc2n(CCOC2CCOCC2)c(=O)n1CC(O)CN1CCc2ccccc2C1] | 34 |
| 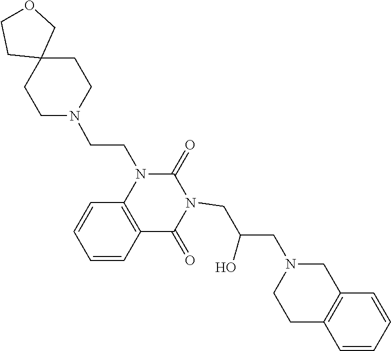 [SMILES: O=c1c2ccccc2n(CCN2CCC3(CCOC3)CC2)c(=O)n1CC(O)CN1CCc2ccccc2C1] | 35 |
| 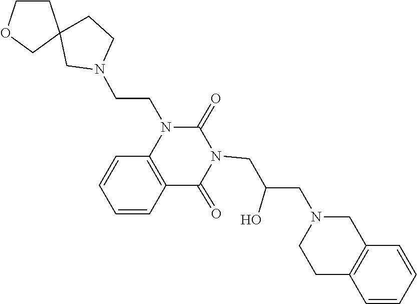 [SMILES: O=c1c2ccccc2n(CCN2CCC3(CCOC3)C2)c(=O)n1CC(O)CN1CCc2ccccc2C1] | 36 |
| 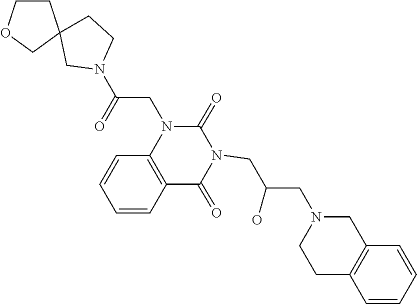 | 37 |
| 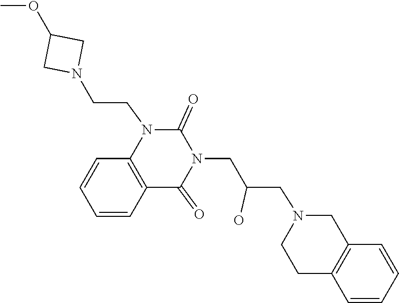 | 38 |
| 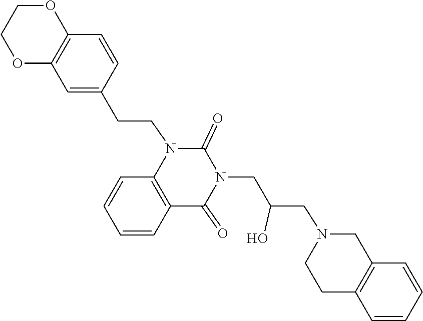 [SMILES: O=c1c2ccccc2n(CCc2ccc3c(c2)OCCO3)c(=O)n1CC(O)CN1CCc2ccccc2C1] | 39 |
| 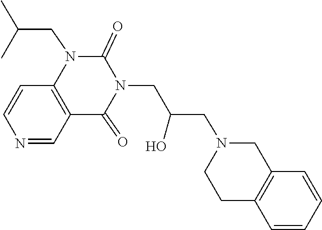 [SMILES: CC(C)Cn1c(=O)n(CC(O)CN2CCc3ccccc3C2)c(=O)c2cnccc21] | 40 |
| 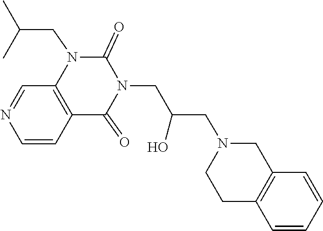 [SMILES: CC(C)Cn1c(=O)n(CC(O)CN2CCc3ccccc3C2)c(=O)c2ccncc21] | 41 |
| 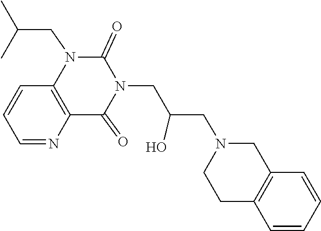 [SMILES: CC(C)Cn1c(=O)n(CC(O)CN2CCc3ccccc3C2)c(=O)c2ncccc21] | 42 |
| 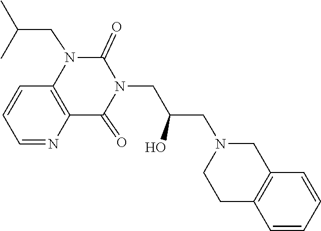 [SMILES: CC(C)Cn1c(=O)n(C[C@H](O)CN2CCc3ccccc3C2)c(=O)c2ncccc21] | 43 |
| 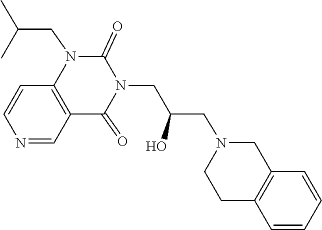 [SMILES: CC(C)Cn1c(=O)n(C[C@H](O)CN2CCc3ccccc3C2)c(=O)c2cnccc21] | 44 |
| 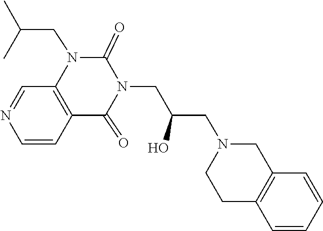 [SMILES: CC(C)Cn1c(=O)n(C[C@H](O)CN2CCc3ccccc3C2)c(=O)c2ccncc21] | 45 |
| 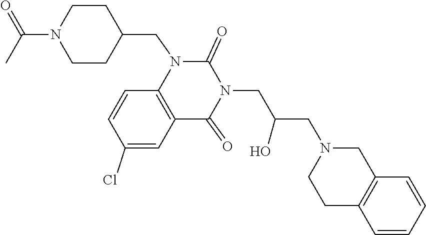 [SMILES: CC(=O)N1CCC(Cn2c(=O)n(CC(O)CN3CCc4ccccc4C3)c(=O)c3cc(Cl)ccc32)CC1] | 46 |
| 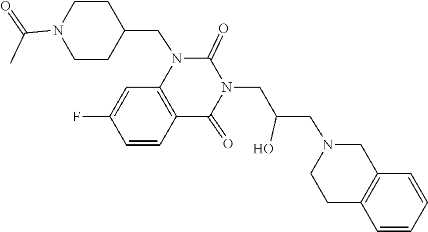 [SMILES: CC(=O)N1CCC(Cn2c(=O)n(CC(O)CN3CCc4ccccc4C3)c(=O)c3ccc(F)cc32)CC1] | 47 |
| 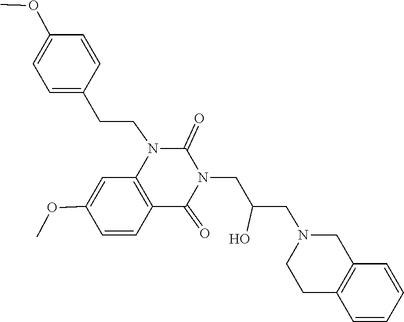 [SMILES: COc1ccc(CCn2c(=O)n(CC(O)CN3CCc4ccccc4C3)c(=O)c3ccc(OC)cc32)cc1] | 48 |
| 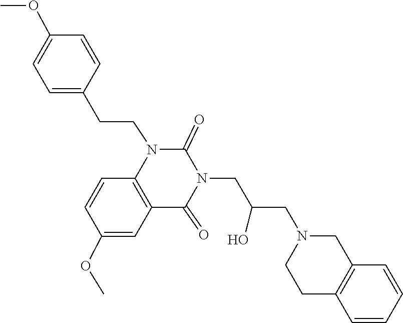 [SMILES: COc1ccc(CCn2c(=O)n(CC(O)CN3CCc4ccccc4C3)c(=O)c3cc(OC)ccc32)cc1] | 49 |
| 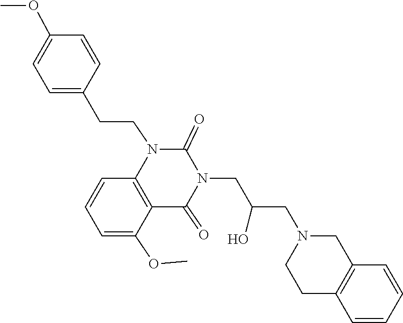 [SMILES: COc1ccc(CCn2c(=O)n(CC(O)CN3CCc4ccccc4C3)c(=O)c3c(OC)cccc32)cc1] | 50 |
| 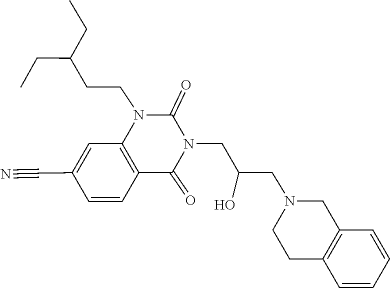 [SMILES: CCC(CC)CCn1c(=O)n(CC(O)CN2CCc3ccccc3C2)c(=O)c2ccc(C#N)cc21] | 51 |
| 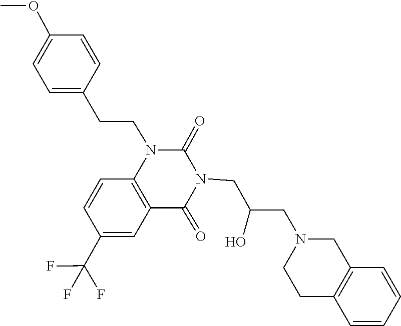 [SMILES: COc1ccc(CCn2c(=O)n(CC(O)CN3CCc4ccccc4C3)c(=O)c3cc(C(F)(F)F)ccc32)cc1] | 52 |
| 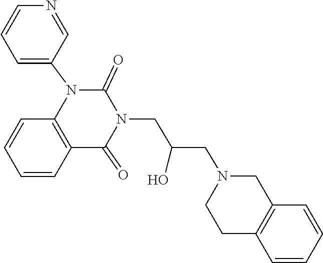 [SMILES: O=c1c2ccccc2n(-c2cccnc2)c(=O)n1CC(O)CN1CCc2ccccc2C1] | 53 |
| 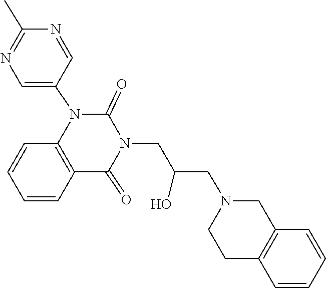 [SMILES: Cc1ncc(-n2c(=O)n(CC(O)CN3CCc4ccccc4C3)c(=O)c3ccccc32)cn1] | 54 |
| 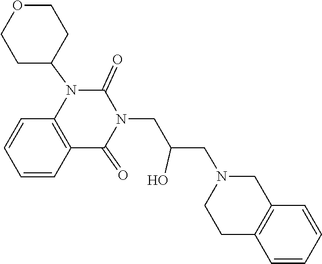 [SMILES: O=c1c2ccccc2n(C2CCOCC2)c(=O)n1CC(O)CN1CCc2ccccc2C1] | 55 |
| 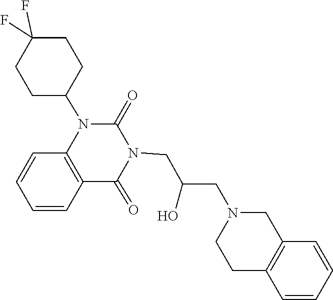 [SMILES: O=c1c2ccccc2n(C2CCC(F)(F)CC2)c(=O)n1CC(O)CN1CCc2ccccc2C1] | 56 |
| 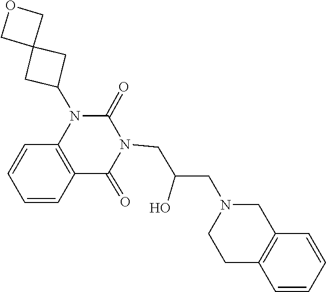 [SMILES: O=c1c2ccccc2n(C2CC3(COC3)C2)c(=O)n1CC(O)CN1CCc2ccccc2C1] | 57 |
| 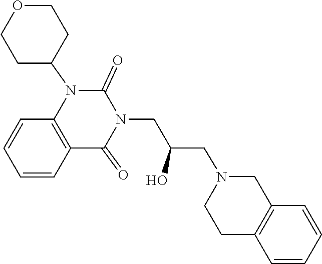 [SMILES: O=c1c2ccccc2n(C2CCOCC2)c(=O)n1C[C@H](O)CN1CCc2ccccc2C1] | 58 |
| 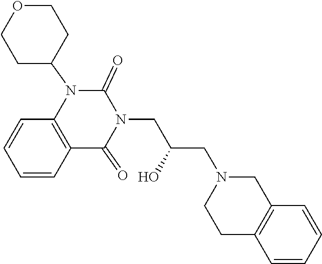 [SMILES: O=c1c2ccccc2n(C2CCOCC2)c(=O)n1C[C@@H](O)CN1CCc2ccccc2C1] | 59 |
| 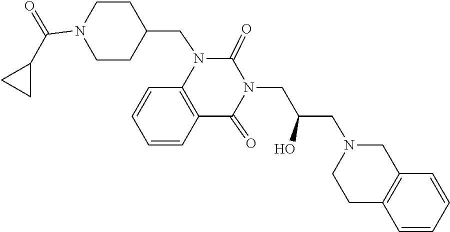 [SMILES: O=C(C1CC1)N1CCC(Cn2c(=O)n(C[C@H](O)CN3CCc4ccccc4C3)c(=O)c3ccccc32)CC1] | 60 |
| 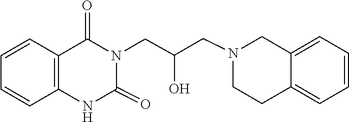 [SMILES: O=c1[nH]c2ccccc2c(=O)n1CC(O)CN1CCc2ccccc2C1] | 61 |
| 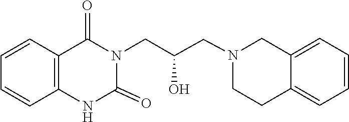 [SMILES: O=c1[nH]c2ccccc2c(=O)n1C[C@@H](O)CN1CCc2ccccc2C1] | 62 |
| 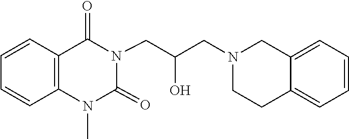 [SMILES: Cn1c(=O)n(CC(O)CN2CCc3ccccc3C2)c(=O)c2ccccc21] | 63 |
| 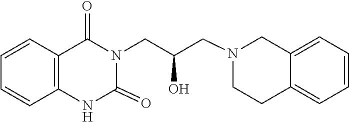 [SMILES: O=c1[nH]c2ccccc2c(=O)n1C[C@H](O)CN1CCc2ccccc2C1] | 64 |
| 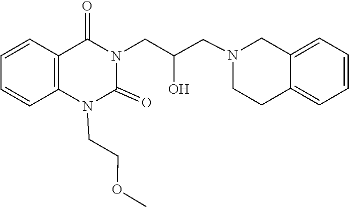 [SMILES: COCCn1c(=O)n(CC(O)CN2CCc3ccccc3C2)c(=O)c2ccccc21] | 65 |
| 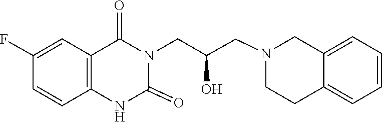 [SMILES: O=c1[nH]c2ccc(F)cc2c(=O)n1C[C@H](O)CN1CCc2ccccc2C1] | 66 |
| 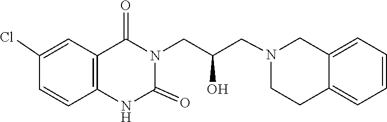 [SMILES: O=c1[nH]c2ccc(Cl)cc2c(=O)n1C[C@H](O)CN1CCc2ccccc2C1] | 67 |
| 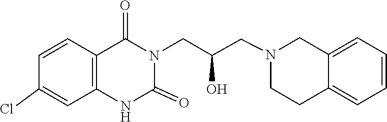 [SMILES: O=c1[nH]c2cc(Cl)ccc2c(=O)n1C[C@H](O)CN1CCc2ccccc2C1] | 68 |
| 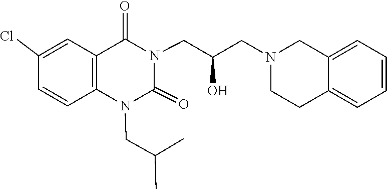 [SMILES: CC(C)Cn1c(=O)n(C[C@H](O)CN2CCc3ccccc3C2)c(=O)c2cc(Cl)ccc21] | 69 |
| 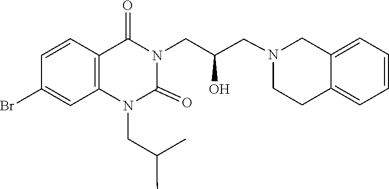 [SMILES: CC(C)Cn1c(=O)n(C[C@H](O)CN2CCc3ccccc3C2)c(=O)c2ccc(Br)cc21] | 70 |
| 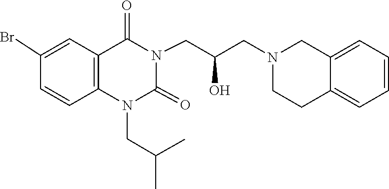 [SMILES: CC(C)Cn1c(=O)n(C[C@H](O)CN2CCc3ccccc3C2)c(=O)c2cc(Br)ccc21] | 71 |
| 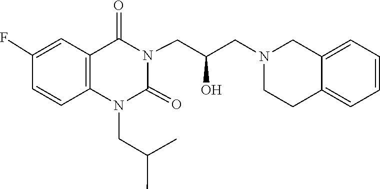 [SMILES: CC(C)Cn1c(=O)n(C[C@H](O)CN2CCc3ccccc3C2)c(=O)c2cc(F)ccc21] | 72 |
| 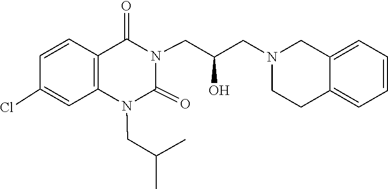 [SMILES: CC(C)Cn1c(=O)n(C[C@H](O)CN2CCc3ccccc3C2)c(=O)c2ccc(Cl)cc21] | 73 |
| 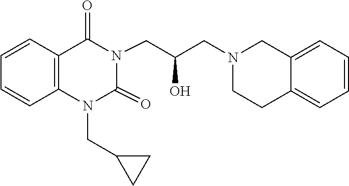 [SMILES: O=c1c2ccccc2n(CC2CC2)c(=O)n1C[C@H](O)CN1CCc2ccccc2C1] | 74 |
| 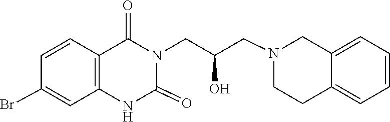 [SMILES: O=c1[nH]c2cc(Br)ccc2c(=O)n1C[C@H](O)CN1CCc2ccccc2C1] | 75 |
| 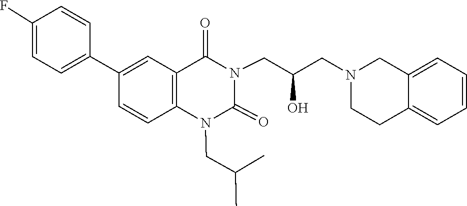 [SMILES: CC(C)Cn1c(=O)n(C[C@H](O)CN2CCc3ccccc3C2)c(=O)c2cc(-c3ccc(F)cc3)ccc21] | 76 |
| 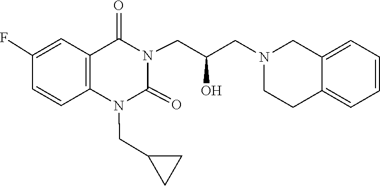 [SMILES: O=c1c2cc(F)ccc2n(CC2CC2)c(=O)n1C[C@H](O)CN1CCc2ccccc2C1] | 77 |
| 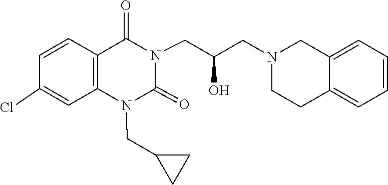 [SMILES: O=c1c2ccc(Cl)cc2n(CC2CC2)c(=O)n1C[C@H](O)CN1CCc2ccccc2C1] | 78 |
| 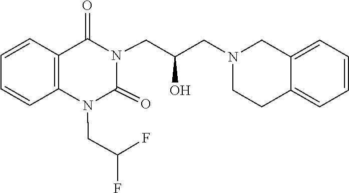 [SMILES: O=c1c2ccccc2n(CC(F)F)c(=O)n1C[C@H](O)CN1CCc2ccccc2C1] | 79 |
| 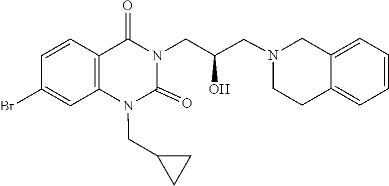 [SMILES: O=c1c2ccc(Br)cc2n(CC2CC2)c(=O)n1C[C@H](O)CN1CCc2ccccc2C1] | 80 |
| 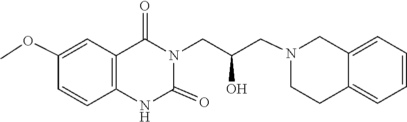 [SMILES: COc1ccc2[nH]c(=O)n(C[C@H](O)CN3CCc4ccccc4C3)c(=O)c2c1] | 81 |
| 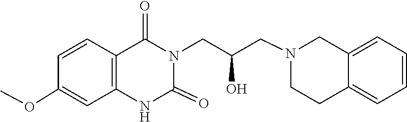 [SMILES: COc1ccc2c(=O)n(C[C@H](O)CN3CCc4ccccc4C3)c(=O)[nH]c2c1] | 82 |
| 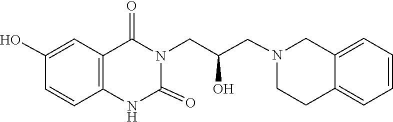 [SMILES: O=c1[nH]c2ccc(O)cc2c(=O)n1C[C@H](O)CN1CCc2ccccc2C1] | 83 |
| 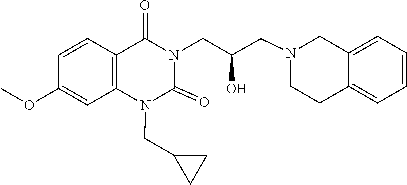 [SMILES: COc1ccc2c(=O)n(C[C@H](O)CN3CCc4ccccc4C3)c(=O)n(CC3CC3)c2c1] | 84 |
| 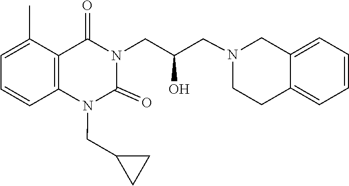 [SMILES: Cc1cccc2c1c(=O)n(C[C@H](O)CN1CCc3ccccc3C1)c(=O)n2CC1CC1] | 85 |
| 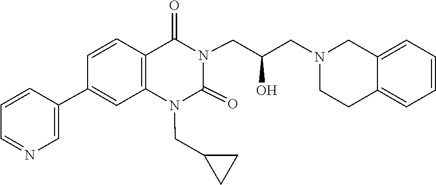 [SMILES: O=c1c2ccc(-c3cccnc3)cc2n(CC2CC2)c(=O)n1C[C@H](O)CN1CCc2ccccc2C1] | 86 |
| 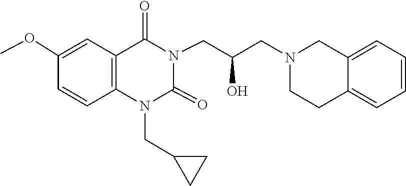 [SMILES: COc1ccc2c(c1)c(=O)n(C[C@H](O)CN1CCc3ccccc3C1)c(=O)n2CC1CC1] | 87 |
| 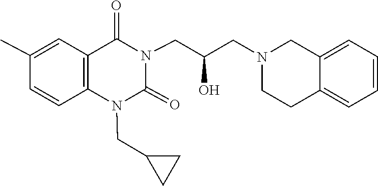 [SMILES: Cc1ccc2c(c1)c(=O)n(C[C@H](O)CN1CCc3ccccc3C1)c(=O)n2CC1CC1] | 88 |
| 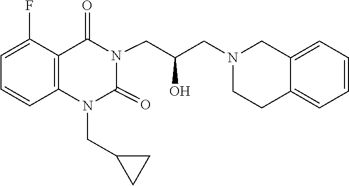 [SMILES: O=c1c2c(F)cccc2n(CC2CC2)c(=O)n1C[C@H](O)CN1CCc2ccccc2C1] | 89 |
| 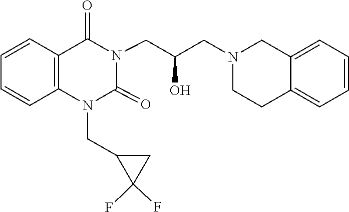 [SMILES: O=c1c2ccccc2n(CC2CC2(F)F)c(=O)n1C[C@H](O)CN1CCc2ccccc2C1] | 90 |
| 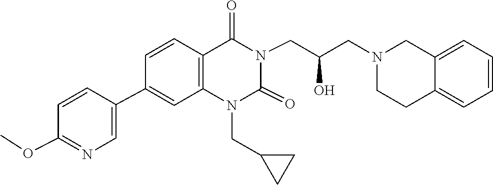 [SMILES: COc1ccc(-c2ccc3c(=O)n(C[C@H](O)CN4CCc5ccccc5C4)c(=O)n(CC4CC4)c3c2)cn1] | 91 |
| 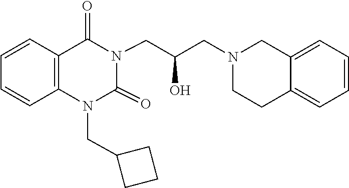 [SMILES: O=c1c2ccccc2n(CC2CCC2)c(=O)n1C[C@H](O)CN1CCc2ccccc2C1] | 92 |
| 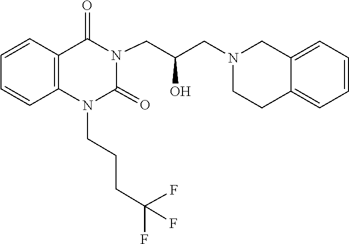 [SMILES: O=c1c2ccccc2n(CCCC(F)(F)F)c(=O)n1C[C@H](O)CN1CCc2ccccc2C1] | 93 |
| 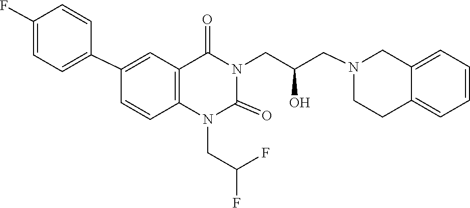 [SMILES: O=c1c2cc(-c3ccc(F)cc3)ccc2n(CC(F)F)c(=O)n1C[C@H](O)CN1CCc2ccccc2C1] | 94 |
| 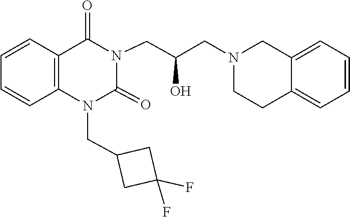 [SMILES: O=c1c2ccccc2n(CC2CC(F)(F)C2)c(=O)n1C[C@H](O)CN1CCc2ccccc2C1] | 95 |
| 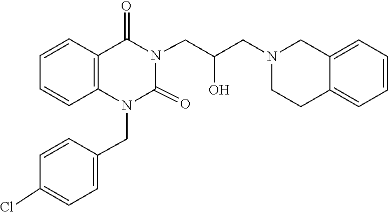 [SMILES: O=c1c2ccccc2n(Cc2ccc(Cl)cc2)c(=O)n1CC(O)CN1CCc2ccccc2C1] | 96 |
| 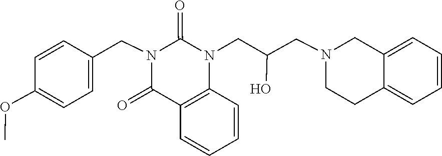 [SMILES: COc1ccc(Cn2c(=O)c3ccccc3n(CC(O)CN3CCc4ccccc4C3)c2=O)cc1] | 97 |
| 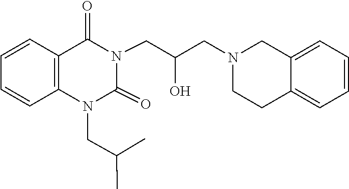 [SMILES: CC(C)Cn1c(=O)n(CC(O)CN2CCc3ccccc3C2)c(=O)c2ccccc21] | 98 |
| 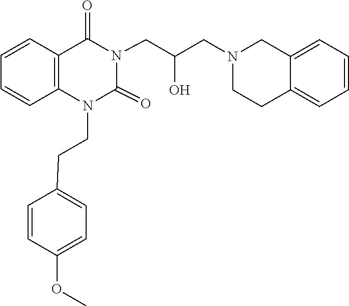 [SMILES: COc1ccc(CCn2c(=O)n(CC(O)CN3CCc4ccccc4C3)c(=O)c3ccccc32)cc1] | 99 |
| 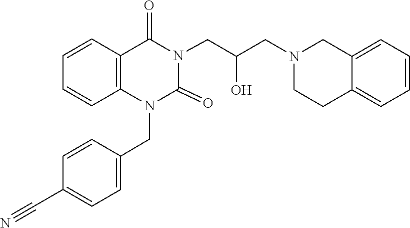 [SMILES: N#Cc1ccc(Cn2c(=O)n(CC(O)CN3CCc4ccccc4C3)c(=O)c3ccccc32)cc1] | 100 |
| 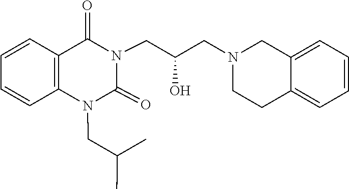 [SMILES: CC(C)Cn1c(=O)n(C[C@@H](O)CN2CCc3ccccc3C2)c(=O)c2ccccc21] | 101 |
| 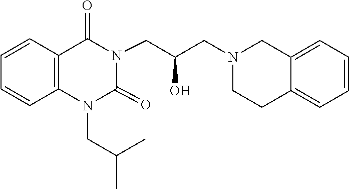 [SMILES: CC(C)Cn1c(=O)n(C[C@H](O)CN2CCc3ccccc3C2)c(=O)c2ccccc21] | 102 |
| 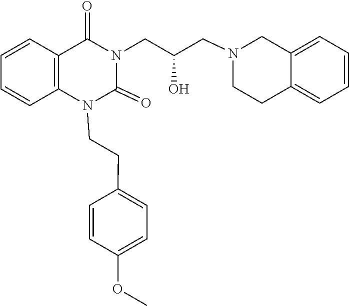 [SMILES: COc1ccc(CCn2c(=O)n(C[C@@H](O)CN3CCc4ccccc4C3)c(=O)c3ccccc32)cc1] | 103 |
| 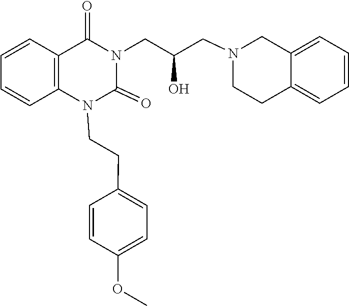 [SMILES: COc1ccc(CCn2c(=O)n(C[C@H](O)CN3CCc4ccccc4C3)c(=O)c3ccccc32)cc1] | 104 |
| 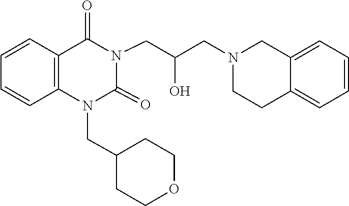 [SMILES: O=c1c2ccccc2n(CC2CCOCC2)c(=O)n1CC(O)CN1CCc2ccccc2C1] | 105 |

4. A pharmaceutical composition comprising a compound according to claim 1.

5. A method for treating a disease state or condition in a subject or patient in need thereof comprising administering a compound according claim 1.

6. A method for treating a disease state or condition in a subject or patient in need thereof comprising administering a pharmaceutical composition according to claim 4.

7. The method of claim 5 wherein the subject or patient is a mammal.

8. The method of claim 7 wherein the mammal is a human.

9. The method of claim 5 wherein the disease state or condition is a cancer.

10. The method of claim 9 wherein the cancer is lymphoma.

11. A method of treating a cancer comprising administering to a mammalian subject in need thereof one or more compounds according to the following formula: 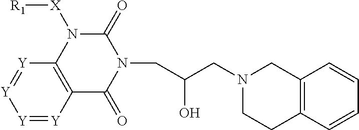
wherein
X is C₁-C₆ alkyl, CO, or a bond,
Y is, at each occurrence, is independently N, CH, or CR².
R¹ is H, C₁-C₆ alkyl, C₁-C₆ heteroalkyl, phenyl, C₃-C₇ heterocycle, C₃-C₇ carbocycle, C₅-C₆ heteroaromatic, C₈-C₁₁ spirocycle, C₈-C₁₁ heterospirocycles, amide, alkoxy, or C₇-C₁₂ fused or bridged bicyclics or heterobicyclics, optionally substituted at any position with one or more R³
R² is H, hydroxy, halo, C₁-C₆ alkyl halide, C₁-C₆ alkoxy, C₁-C₆ alkyl, C₁-C₆ alkenyl, C₁-C₆ alkynyl, C₁-C₆ heteroalkyl, C₁-C₆ heteroalkenyl, C₁-C₆ heteroalkynyl, amino, alkyl amide, alkyl carboxylic acid, pyridyl, methoxy pyridyl, phenyl, fluorophenyl, and cyano, or a pharmaceutically acceptable salt, ester, prodrug, or solvate thereof
wherein R¹ optionally has the formula A¹-L¹-B¹-, where A¹ is a first aromatic, heteroaromatic, carbocycle, heterocycle, spirocycle, or bridged or fused combinations thereof, where L¹ is a linker selected from alkyl, heteroalkyl, CO, O, N, or S, and where B¹ is a second aromatic, heteroaromatic, carbocycle, heterocycle, spirocycle, heterospirocycle, or bridged or fused combinations thereof, wherein B¹ is attached to the N of the remainder of the compound
wherein R³ is selected from H, halo, hydroxy, acyl, —C═OR⁴, alkyl halide, alkoxy, alkyl, alkenyl, alkynyl, heteroalkyl, heteroalkenyl, heteroalkynyl, amino, alkyl amide, alkyl carboxylic acid, and cyano, each optionally connected through a C₁-C₆ linker, and
wherein R⁴ is H or a C₁-C₆ alkyl, C₁-C₆ heteroalkyl, aryl, or heteroaryl, or a pharmaceutically acceptable salt, ester, prodrug, or solvate thereof.

12. The method of claim 11 wherein the cancer is lymphoma.

13. The method of claim 11 wherein the mammalian subject is a human.

14. The method of claim 11 wherein the one or more compounds are selected from compounds 1-105 as shown in the table below:

| | |
|---|---|
| 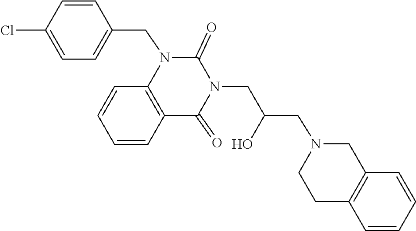 [SMILES: O=c1c2ccccc2n(Cc2ccc(Cl)cc2)c(=O)n1CC(O)CN1CCc2ccccc2C1] | 1 |
| 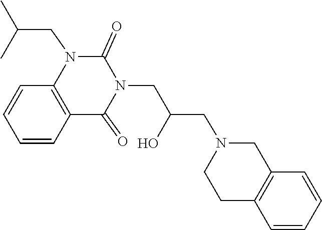 [SMILES: CC(C)Cn1c(=O)n(CC(O)CN2CCc3ccccc3C2)c(=O)c2ccccc21] | 2 |
| 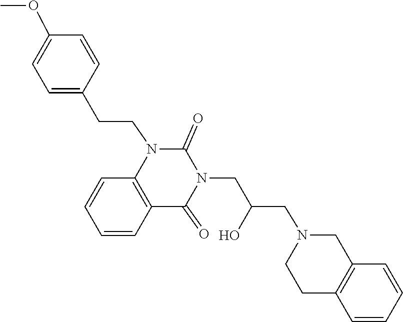 [SMILES: COc1ccc(CCn2c(=O)n(CC(O)CN3CCc4ccccc4C3)c(=O)c3ccccc32)cc1] | 3 |
| 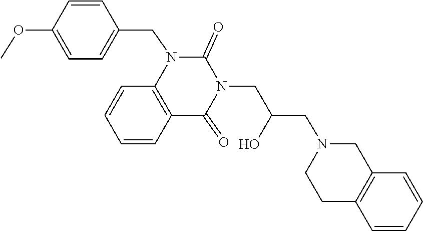 [SMILES: COc1ccc(Cn2c(=O)n(CC(O)CN3CCc4ccccc4C3)c(=O)c3ccccc32)cc1] | 4 |
| 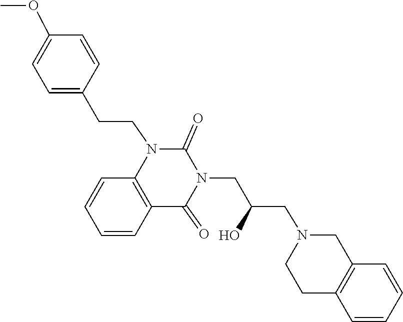 [SMILES: COc1ccc(CCn2c(=O)n(C[C@H](O)CN3CCc4ccccc4C3)c(=O)c3ccccc32)cc1] | 5 |
| 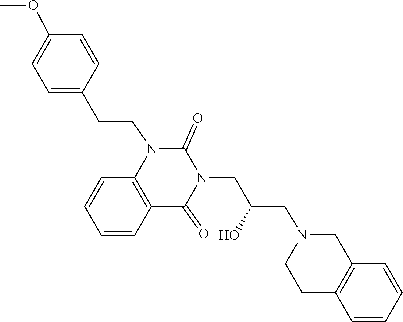 [SMILES: COc1ccc(CCn2c(=O)n(C[C@@H](O)CN3CCc4ccccc4C3)c(=O)c3ccccc32)cc1] | 6 |
| 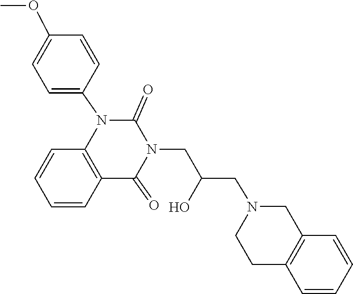 [SMILES: COc1ccc(-n2c(=O)n(CC(O)CN3CCc4ccccc4C3)c(=O)c3ccccc32)cc1] | 7 |
| 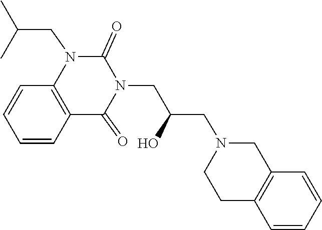 [SMILES: CC(C)Cn1c(=O)n(C[C@H](O)CN2CCc3ccccc3C2)c(=O)c2ccccc21] | 8 |
| 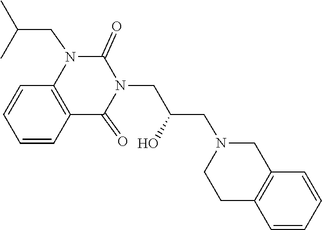 [SMILES: CC(C)Cn1c(=O)n(C[C@@H](O)CN2CCc3ccccc3C2)c(=O)c2ccccc21] | 9 |
| 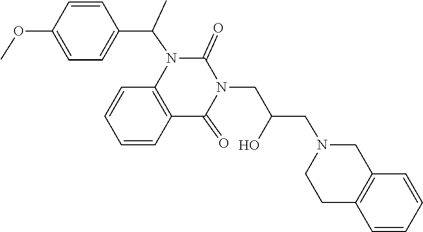 [SMILES: COc1ccc(C(C)n2c(=O)n(CC(O)CN3CCc4ccccc4C3)c(=O)c3ccccc32)cc1] | 10 |
| 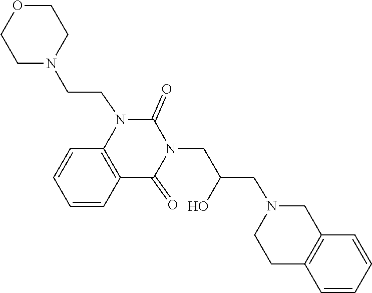 [SMILES: O=c1c2ccccc2n(CCN2CCOCC2)c(=O)n1CC(O)CN1CCc2ccccc2C1] | 11 |
| 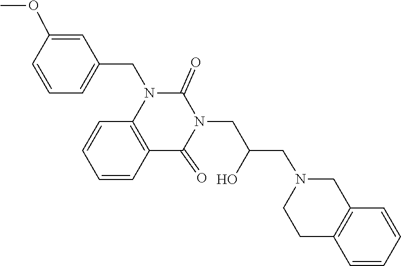 [SMILES: COc1cccc(Cn2c(=O)n(CC(O)CN3CCc4ccccc4C3)c(=O)c3ccccc32)c1] | 12 |
| 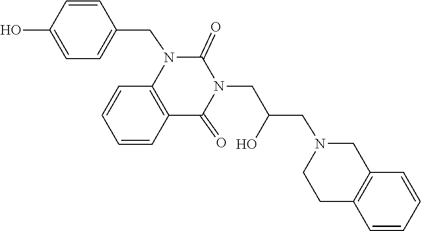 [SMILES: O=c1c2ccccc2n(Cc2ccc(O)cc2)c(=O)n1CC(O)CN1CCc2ccccc2C1] | 13 |
| 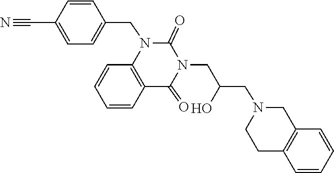 [SMILES: N#Cc1ccc(Cn2c(=O)n(CC(O)CN3CCc4ccccc4C3)c(=O)c3ccccc32)cc1] | 14 |
| 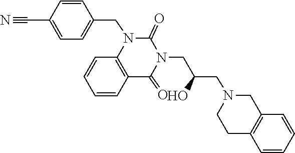 [SMILES: N#Cc1ccc(Cn2c(=O)n(C[C@H](O)CN3CCc4ccccc4C3)c(=O)c3ccccc32)cc1] | 15 |
| 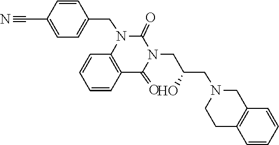 [SMILES: N#Cc1ccc(Cn2c(=O)n(C[C@@H](O)CN3CCc4ccccc4C3)c(=O)c3ccccc32)cc1] | 16 |
| 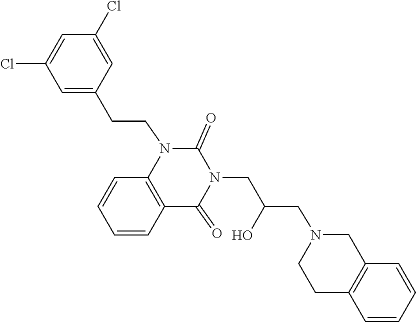 [SMILES: O=c1c2ccccc2n(CCc2cc(Cl)cc(Cl)c2)c(=O)n1CC(O)CN1CCc2ccccc2C1] | 17 |
| 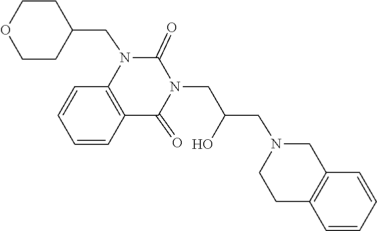 [SMILES: O=c1c2ccccc2n(CC2CCOCC2)c(=O)n1CC(O)CN1CCc2ccccc2C1] | 18 |
| 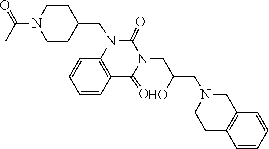 [SMILES: CC(=O)N1CCC(Cn2c(=O)n(CC(O)CN3CCc4ccccc4C3)c(=O)c3ccccc32)CC1] | 19 |
| 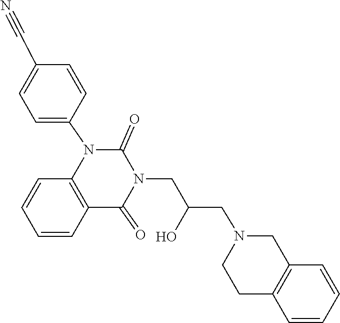 [SMILES: N#Cc1ccc(-n2c(=O)n(CC(O)CN3CCc4ccccc4C3)c(=O)c3ccccc32)cc1] | 20 |
| 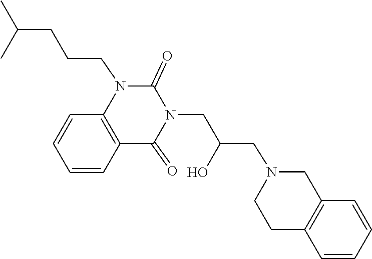 [SMILES: CC(C)CCCn1c(=O)n(CC(O)CN2CCc3ccccc3C2)c(=O)c2ccccc21] | 21 |
| 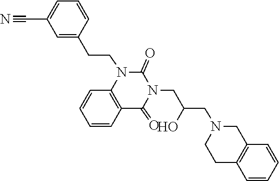 [SMILES: N#Cc1cccc(CCn2c(=O)n(CC(O)CN3CCc4ccccc4C3)c(=O)c3ccccc32)c1] | 22 |
| 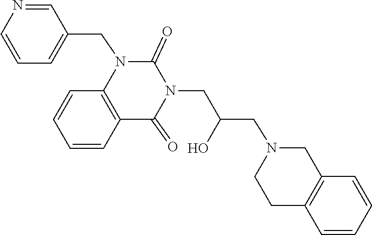 [SMILES: O=c1c2ccccc2n(Cc2cccnc2)c(=O)n1CC(O)CN1CCc2ccccc2C1] | 23 |
| 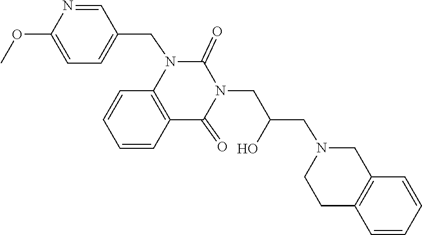 [SMILES: COc1ccc(Cn2c(=O)n(CC(O)CN3CCc4ccccc4C3)c(=O)c3ccccc32)cn1] | 24 |
| 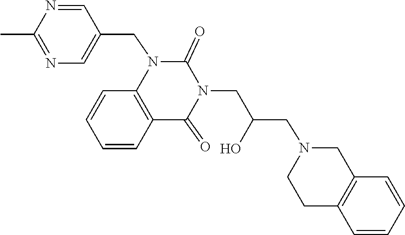 [SMILES: Cc1ncc(Cn2c(=O)n(CC(O)CN3CCc4ccccc4C3)c(=O)c3ccccc32)cn1] | 25 |
| 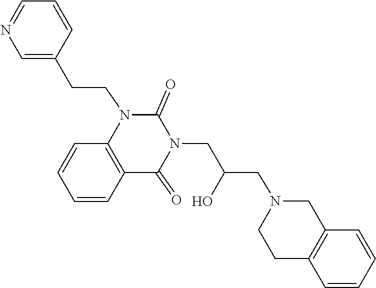 [SMILES: O=c1c2ccccc2n(CCc2cccnc2)c(=O)n1CC(O)CN1CCc2ccccc2C1] | 26 |
| 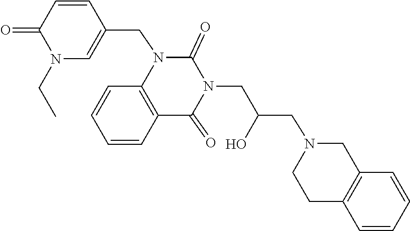 [SMILES: CCn1cc(Cn2c(=O)n(CC(O)CN3CCc4ccccc4C3)c(=O)c3ccccc32)ccc1=O] | 27 |
| 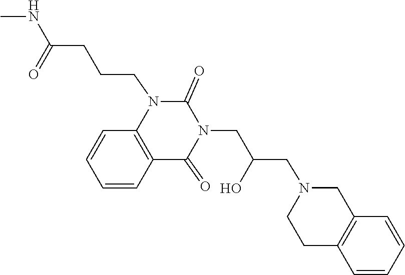 [SMILES: CNC(=O)CCCn1c(=O)n(CC(O)CN2CCc3ccccc3C2)c(=O)c2ccccc21] | 28 |
| 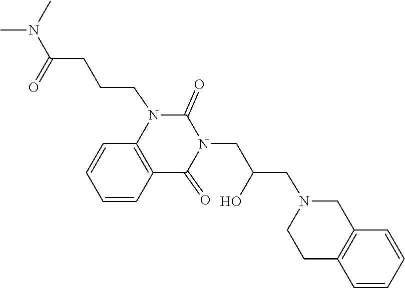 [SMILES: CN(C)C(=O)CCCn1c(=O)n(CC(O)CN2CCc3ccccc3C2)c(=O)c2ccccc21] | 29 |
| 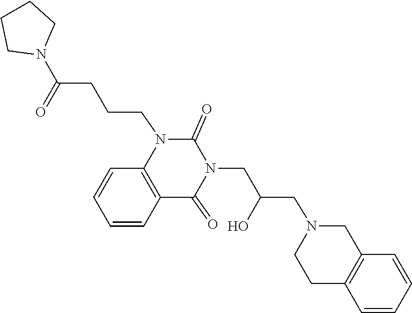 [SMILES: O=C(CCCn1c(=O)n(CC(O)CN2CCc3ccccc3C2)c(=O)c2ccccc21)N1CCCC1] | 30 |
| 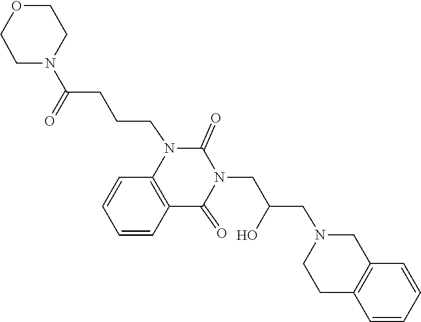 [SMILES: O=C(CCCn1c(=O)n(CC(O)CN2CCc3ccccc3C2)c(=O)c2ccccc21)N1CCOCC1] | 31 |
| 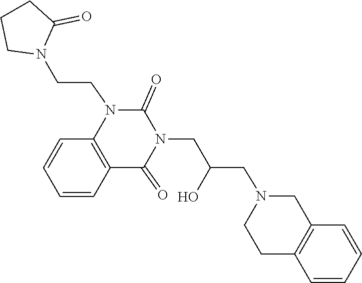 [SMILES: O=C1CCCN1CCn1c(=O)n(CC(O)CN2CCc3ccccc3C2)c(=O)c2ccccc21] | 32 |
| 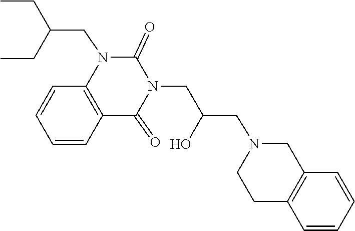 [SMILES: CCC(CC)Cn1c(=O)n(CC(O)CN2CCc3ccccc3C2)c(=O)c2ccccc21] | 33 |
| 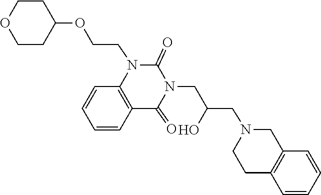 [SMILES: O=c1c2ccccc2n(CCOC2CCOCC2)c(=O)n1CC(O)CN1CCc2ccccc2C1] | 34 |
| 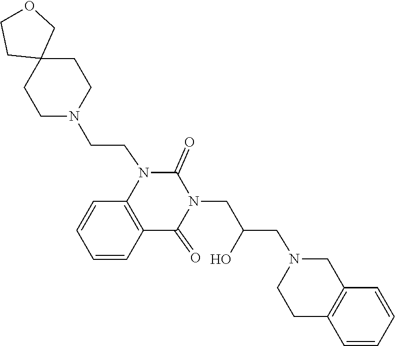 [SMILES: O=c1c2ccccc2n(CCN2CCC3(CCOC3)CC2)c(=O)n1CC(O)CN1CCc2ccccc2C1] | 35 |
| 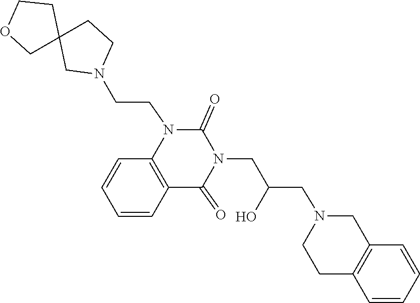 [SMILES: O=c1c2ccccc2n(CCN2CCC3(CCOC3)C2)c(=O)n1CC(O)CN1CCc2ccccc2C1] | 36 |
| 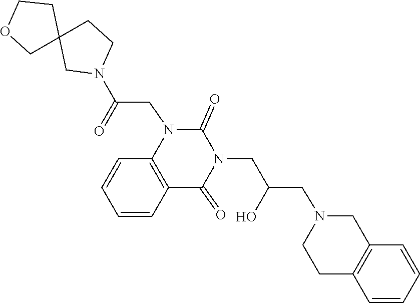 [SMILES: O=C(Cn1c(=O)n(CC(O)CN2CCc3ccccc3C2)c(=O)c2ccccc21)N1CCC2(CCOC2)C1] | 37 |
| 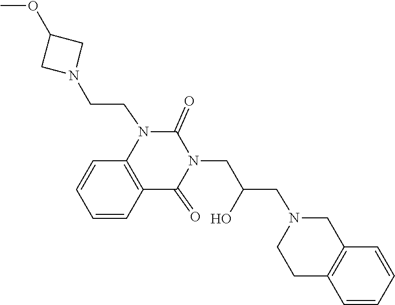 [SMILES: COC1CN(CCn2c(=O)n(CC(O)CN3CCc4ccccc4C3)c(=O)c3ccccc32)C1] | 38 |
| 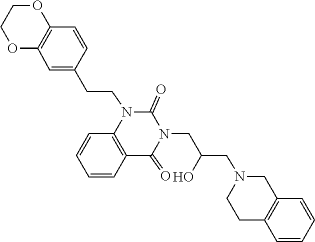 [SMILES: O=c1c2ccccc2n(CCc2ccc3c(c2)OCCO3)c(=O)n1CC(O)CN1CCc2ccccc2C1] | 39 |
| 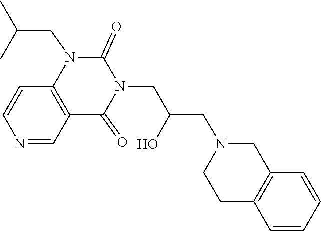 [SMILES: CC(C)Cn1c(=O)n(CC(O)CN2CCc3ccccc3C2)c(=O)c2cnccc21] | 40 |
| 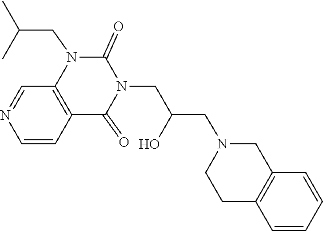 [SMILES: CC(C)Cn1c(=O)n(CC(O)CN2CCc3ccccc3C2)c(=O)c2ccncc21] | 41 |
| 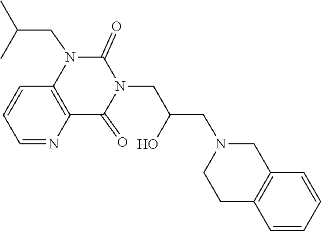 [SMILES: CC(C)Cn1c(=O)n(CC(O)CN2CCc3ccccc3C2)c(=O)c2ncccc21] | 42 |
| 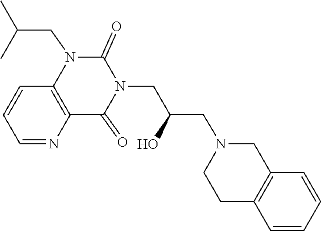 [SMILES: CC(C)Cn1c(=O)n(C[C@H](O)CN2CCc3ccccc3C2)c(=O)c2ncccc21] | 43 |
| 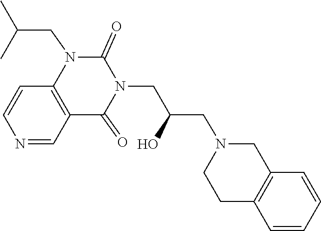 [SMILES: CC(C)Cn1c(=O)n(C[C@H](O)CN2CCc3ccccc3C2)c(=O)c2cnccc21] | 44 |
| 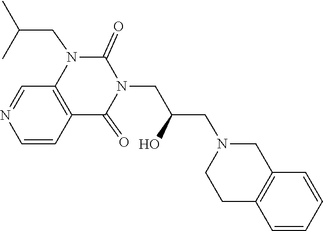 [SMILES: CC(C)Cn1c(=O)n(C[C@H](O)CN2CCc3ccccc3C2)c(=O)c2ccncc21] | 45 |
| 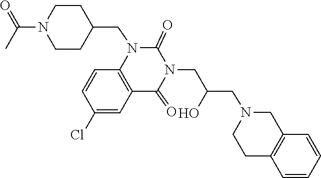 [SMILES: CC(=O)N1CCC(Cn2c(=O)n(CC(O)CN3CCc4ccccc4C3)c(=O)c3cc(Cl)ccc32)CC1] | 46 |
| 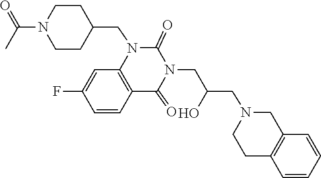 [SMILES: CC(=O)N1CCC(Cn2c(=O)n(CC(O)CN3CCc4ccccc4C3)c(=O)c3ccc(F)cc32)CC1] | 47 |
| 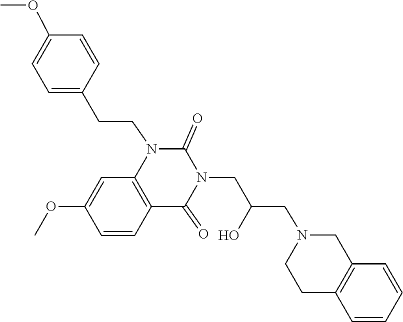 [SMILES: COc1ccc(CCn2c(=O)n(CC(O)CN3CCc4ccccc4C3)c(=O)c3ccc(OC)cc32)cc1] | 48 |
| 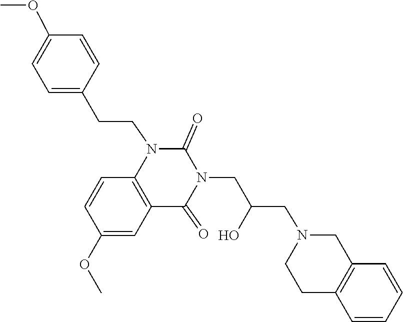 [SMILES: COc1ccc(CCn2c(=O)n(CC(O)CN3CCc4ccccc4C3)c(=O)c3cc(OC)ccc32)cc1] | 49 |
| 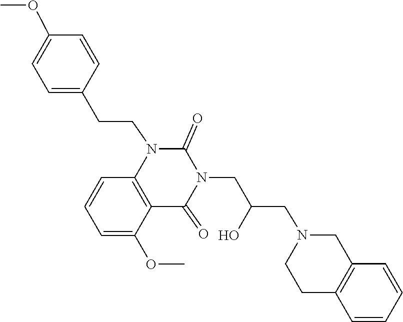 [SMILES: COc1ccc(CCn2c(=O)n(CC(O)CN3CCc4ccccc4C3)c(=O)c3c(OC)cccc32)cc1] | 50 |
| 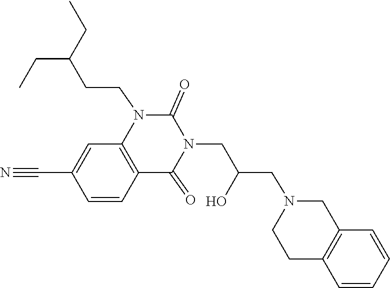 [SMILES: CCC(CC)CCn1c(=O)n(CC(O)CN2CCc3ccccc3C2)c(=O)c2ccc(C#N)cc21] | 51 |
| 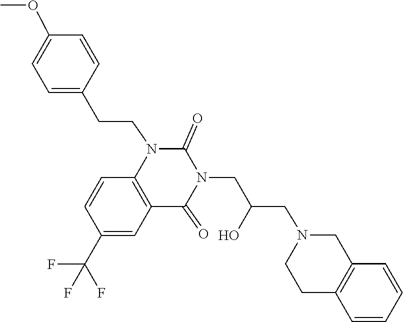 [SMILES: COc1ccc(CCn2c(=O)n(CC(O)CN3CCc4ccccc4C3)c(=O)c3cc(C(F)(F)F)ccc32)cc1] | 52 |
| 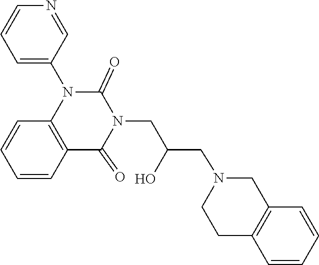 [SMILES: O=c1c2ccccc2n(-c2cccnc2)c(=O)n1CC(O)CN1CCc2ccccc2C1] | 53 |
| 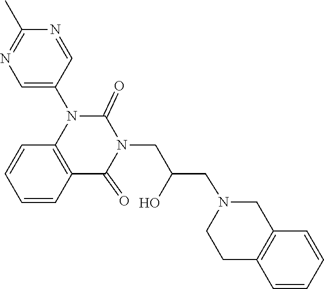 [SMILES: Cc1ncc(-n2c(=O)n(CC(O)CN3CCc4ccccc4C3)c(=O)c3ccccc32)cn1] | 54 |
| 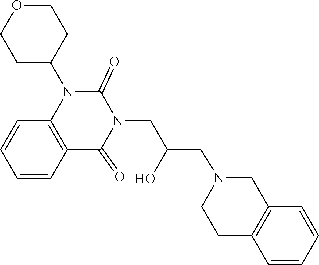 [SMILES: O=c1c2ccccc2n(C2CCOCC2)c(=O)n1CC(O)CN1CCc2ccccc2C1] | 55 |
| 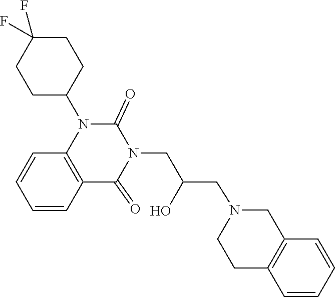 [SMILES: O=c1c2ccccc2n(C2CCC(F)(F)CC2)c(=O)n1CC(O)CN1CCc2ccccc2C1] | 56 |
| 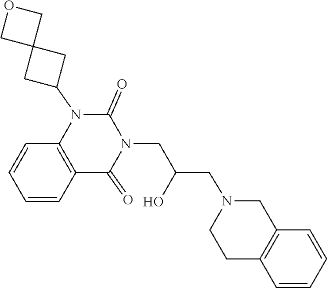 [SMILES: O=c1c2ccccc2n(C2CC3(COC3)C2)c(=O)n1CC(O)CN1CCc2ccccc2C1] | 57 |
| 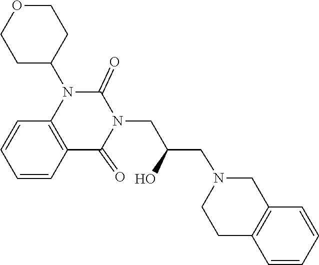 [SMILES: O=c1c2ccccc2n(C2CCOCC2)c(=O)n1C[C@H](O)CN1CCc2ccccc2C1] | 58 |
| 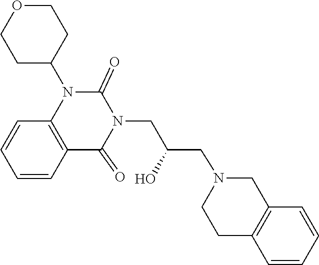 [SMILES: O=c1c2ccccc2n(C2CCOCC2)c(=O)n1C[C@@H](O)CN1CCc2ccccc2C1] | 59 |
| 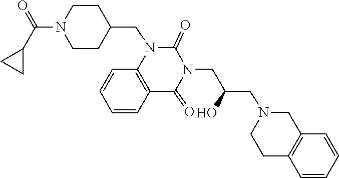 [SMILES: O=C(C1CC1)N1CCC(Cn2c(=O)n(C[C@H](O)CN3CCc4ccccc4C3)c(=O)c3ccccc32)CC1] | 60 |
| 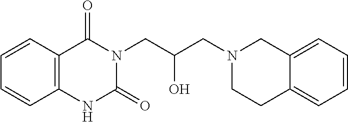 [SMILES: O=c1[nH]c2ccccc2c(=O)n1CC(O)CN1CCc2ccccc2C1] | 61 |
| 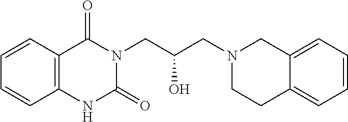 [SMILES: O=c1[nH]c2ccccc2c(=O)n1C[C@@H](O)CN1CCc2ccccc2C1] | 62 |
| 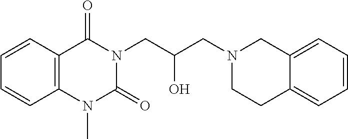 [SMILES: Cn1c(=O)n(CC(O)CN2CCc3ccccc3C2)c(=O)c2ccccc21] | 63 |
| 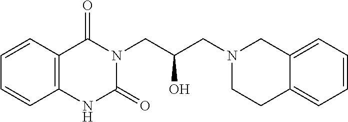 [SMILES: O=c1[nH]c2ccccc2c(=O)n1C[C@H](O)CN1CCc2ccccc2C1] | 64 |
| 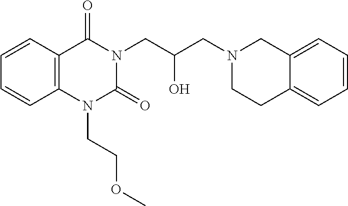 [SMILES: COCCn1c(=O)n(CC(O)CN2CCc3ccccc3C2)c(=O)c2ccccc21] | 65 |
| 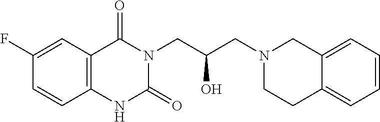 [SMILES: O=c1[nH]c2ccc(F)cc2c(=O)n1C[C@H](O)CN1CCc2ccccc2C1] | 66 |
| 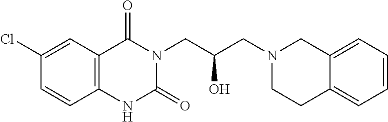 [SMILES: O=c1[nH]c2ccc(Cl)cc2c(=O)n1C[C@H](O)CN1CCc2ccccc2C1] | 67 |
| 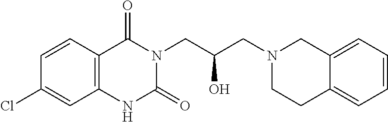 [SMILES: O=c1[nH]c2cc(Cl)ccc2c(=O)n1C[C@H](O)CN1CCc2ccccc2C1] | 68 |
| 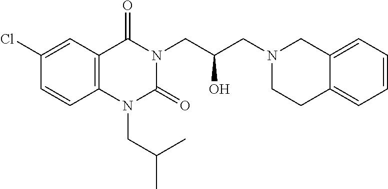 [SMILES: CC(C)Cn1c(=O)n(C[C@H](O)CN2CCc3ccccc3C2)c(=O)c2cc(Cl)ccc21] | 69 |
| 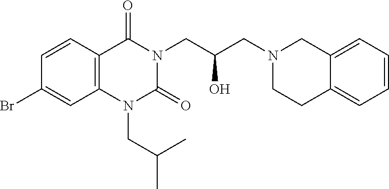 [SMILES: CC(C)Cn1c(=O)n(C[C@H](O)CN2CCc3ccccc3C2)c(=O)c2ccc(Br)cc21] | 70 |
| 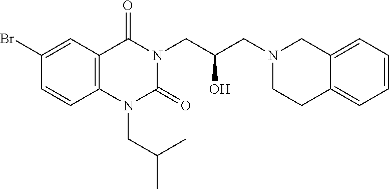 [SMILES: CC(C)Cn1c(=O)n(C[C@H](O)CN2CCc3ccccc3C2)c(=O)c2cc(Br)ccc21] | 71 |
| 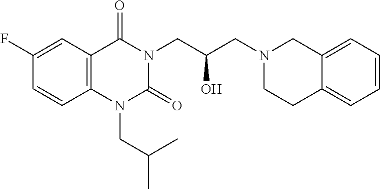 [SMILES: CC(C)Cn1c(=O)n(C[C@H](O)CN2CCc3ccccc3C2)c(=O)c2cc(F)ccc21] | 72 |
| 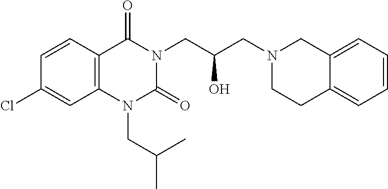 [SMILES: CC(C)Cn1c(=O)n(C[C@H](O)CN2CCc3ccccc3C2)c(=O)c2ccc(Cl)cc21] | 73 |
| 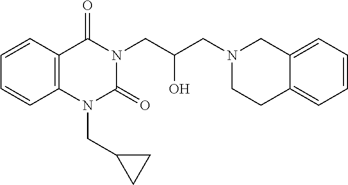 [SMILES: O=c1c2ccccc2n(CC2CC2)c(=O)n1CC(O)CN1CCc2ccccc2C1] | 74 |
| 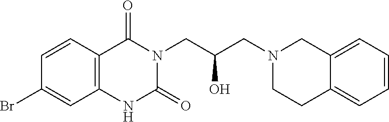 [SMILES: O=c1[nH]c2cc(Br)ccc2c(=O)n1C[C@H](O)CN1CCc2ccccc2C1] | 75 |
| 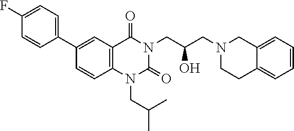 [SMILES: CC(C)Cn1c(=O)n(C[C@H](O)CN2CCc3ccccc3C2)c(=O)c2cc(-c3ccc(F)cc3)ccc21] | 76 |
| 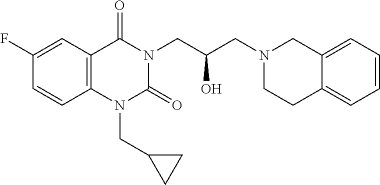 [SMILES: O=c1c2cc(F)ccc2n(CC2CC2)c(=O)n1C[C@H](O)CN1CCc2ccccc2C1] | 77 |
| 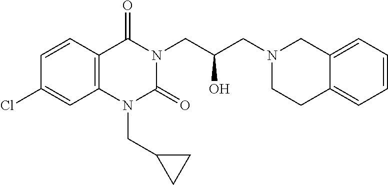 [SMILES: O=c1c2ccc(Cl)cc2n(CC2CC2)c(=O)n1C[C@H](O)CN1CCc2ccccc2C1] | 78 |
| 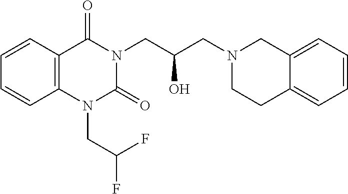 [SMILES: O=c1c2ccccc2n(CC(F)F)c(=O)n1C[C@H](O)CN1CCc2ccccc2C1] | 79 |
| 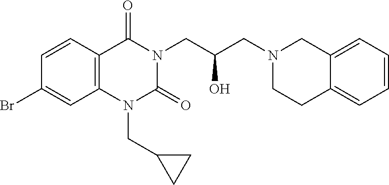 [SMILES: O=c1c2ccc(Br)cc2n(CC2CC2)c(=O)n1C[C@H](O)CN1CCc2ccccc2C1] | 80 |
| 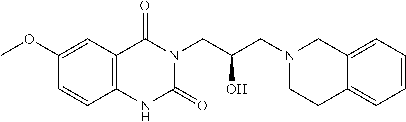 [SMILES: COc1ccc2[nH]c(=O)n(C[C@H](O)CN3CCc4ccccc4C3)c(=O)c2c1] | 81 |
| 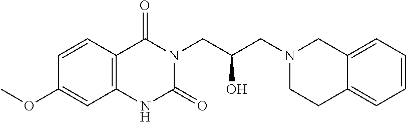 [SMILES: COc1ccc2c(=O)n(C[C@H](O)CN3CCc4ccccc4C3)c(=O)[nH]c2c1] | 82 |
| 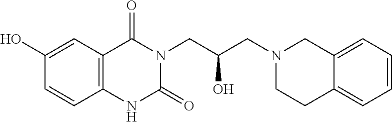 [SMILES: O=c1[nH]c2ccc(O)cc2c(=O)n1C[C@H](O)CN1CCc2ccccc2C1] | 83 |
| 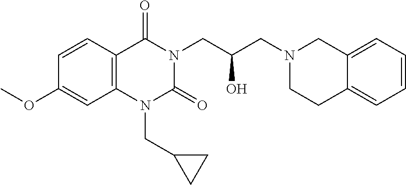 [SMILES: COc1ccc2c(=O)n(C[C@H](O)CN3CCc4ccccc4C3)c(=O)n(CC3CC3)c2c1] | 84 |
| 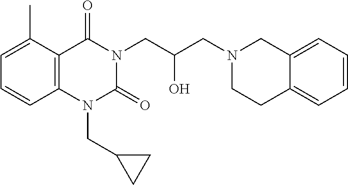 [SMILES: Cc1cccc2c1c(=O)n(CC(O)CN1CCc3ccccc3C1)c(=O)n2CC1CC1] | 85 |
| 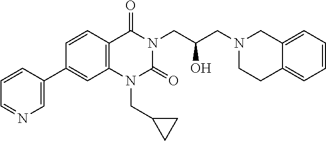 [SMILES: O=c1c2ccc(-c3cccnc3)cc2n(CC2CC2)c(=O)n1C[C@H](O)CN1CCc2ccccc2C1] | 86 |
| 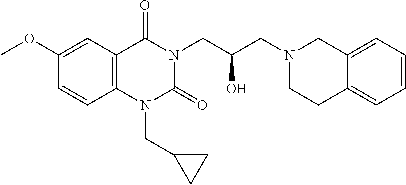 [SMILES: COc1ccc2c(c1)c(=O)n(C[C@H](O)CN1CCc3ccccc3C1)c(=O)n2CC1CC1] | 87 |
| 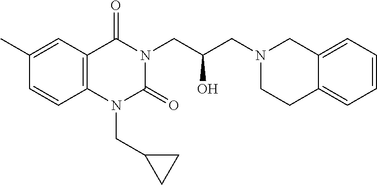 [SMILES: Cc1ccc2c(c1)c(=O)n(C[C@H](O)CN1CCc3ccccc3C1)c(=O)n2CC1CC1] | 88 |
| 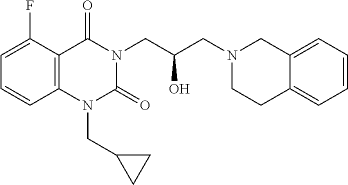 [SMILES: O=c1c2c(F)cccc2n(CC2CC2)c(=O)n1C[C@H](O)CN1CCc2ccccc2C1] | 89 |
| 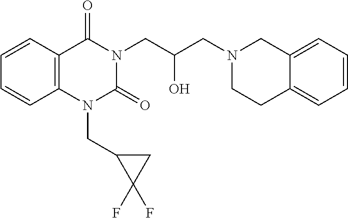 [SMILES: O=c1c2ccccc2n(CC2CC2(F)F)c(=O)n1CC(O)CN1CCc2ccccc2C1] | 90 |
| 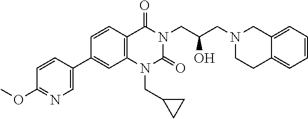 [SMILES: COc1ccc(-c2ccc3c(=O)n(C[C@H](O)CN4CCc5ccccc5C4)c(=O)n(CC4CC4)c3c2)cn1] | 91 |
| 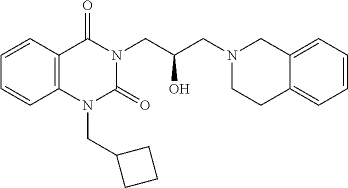 [SMILES: O=c1c2ccccc2n(CC2CCC2)c(=O)n1C[C@H](O)CN1CCc2ccccc2C1] | 92 |
| 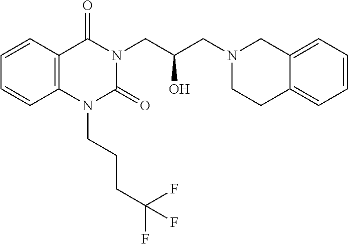 [SMILES: O=c1c2ccccc2n(CCCC(F)(F)F)c(=O)n1C[C@H](O)CN1CCc2ccccc2C1] | 93 |
| 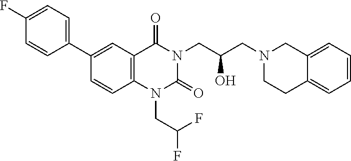 [SMILES: O=c1c2cc(-c3ccc(F)cc3)ccc2n(CC(F)F)c(=O)n1C[C@H](O)CN1CCc2ccccc2C1] | 94 |
| 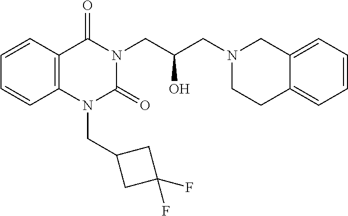 [SMILES: O=c1c2ccccc2n(CC2CC(F)(F)C2)c(=O)n1C[C@H](O)CN1CCc2ccccc2C1] | 95 |
| 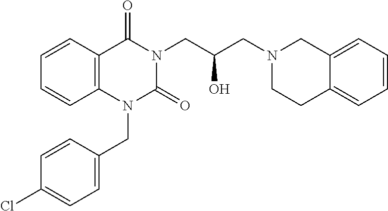 [SMILES: O=c1c2ccccc2n(Cc2ccc(Cl)cc2)c(=O)n1C[C@H](O)CN1CCc2ccccc2C1] | 96 |
| 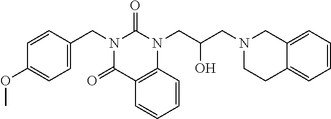 [SMILES: COc1ccc(Cn2c(=O)c3ccccc3n(CC(O)CN3CCc4ccccc4C3)c2=O)cc1] | 97 |
| 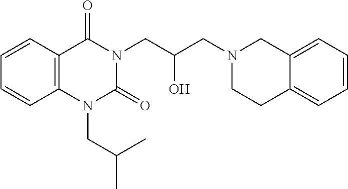 [SMILES: CC(C)Cn1c(=O)n(CC(O)CN2CCc3ccccc3C2)c(=O)c2ccccc21] | 98 |
| 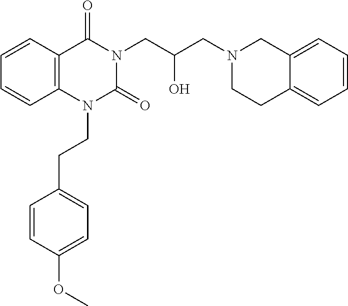 [SMILES: COc1ccc(CCn2c(=O)n(CC(O)CN3CCc4ccccc4C3)c(=O)c3ccccc32)cc1] | 99 |
| 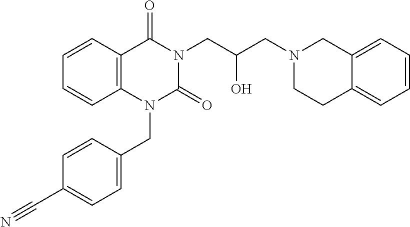 [SMILES: N#Cc1ccc(Cn2c(=O)n(CC(O)CN3CCc4ccccc4C3)c(=O)c3ccccc32)cc1] | 100 |
| 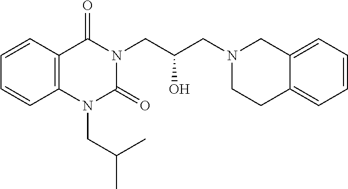 [SMILES: CC(C)Cn1c(=O)n(C[C@@H](O)CN2CCc3ccccc3C2)c(=O)c2ccccc21] | 101 |
| 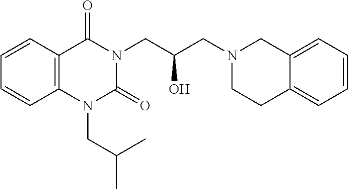 [SMILES: CC(C)Cn1c(=O)n(C[C@H](O)CN2CCc3ccccc3C2)c(=O)c2ccccc21] | 102 |
| 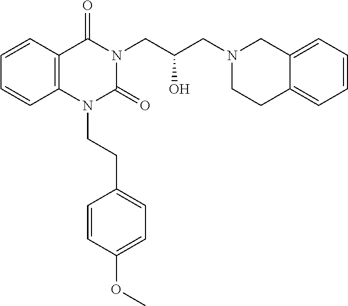 [SMILES: COc1ccc(CCn2c(=O)n(C[C@@H](O)CN3CCc4ccccc4C3)c(=O)c3ccccc32)cc1] | 103 |
| 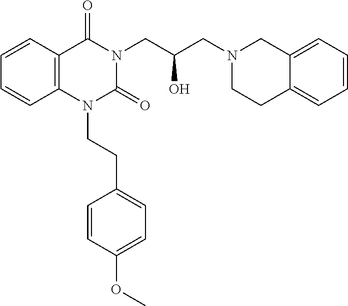 [SMILES: COc1ccc(CCn2c(=O)n(C[C@H](O)CN3CCc4ccccc4C3)c(=O)c3ccccc32)cc1] | 104 |
| 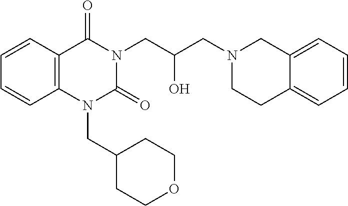 [SMILES: O=c1c2ccccc2n(CC2CCOCC2)c(=O)n1CC(O)CN1CCc2ccccc2C1] | 105 |
